(19) United States
(12) Patent Application Publication
(10) Pub. No.: US 2024/0415773 A1
(43) Pub. Date: Dec. 19, 2024

(54) COMPOSITIONS AND METHODS FOR TREATMENT OF IMMUNE CHECKPOINT RESISTANT CANCERS

(71) Applicant: The George Washington University, Washington, DC (US)

(72) Inventors: Rohan Fernandes, Washington, DC (US); Elizabeth Sweeney, Washington, DC (US); Preethi Bala Balakrishnan, Washington, DC (US); Jacob Medina, Washington, DC (US)

(21) Appl. No.: 18/814,236

(22) Filed: Aug. 23, 2024

Related U.S. Application Data
(63) Continuation of application No. 17/714,010, filed on Apr. 5, 2022.
(60) Provisional application No. 63/171,452, filed on Apr. 6, 2021.

(51) Int. Cl. A61K 9/14; A61K 9/28; A61K 9/51; A61N 5/067; A61P 35/00

(52) U.S. Cl. CPC A61K 9/143; A61K 9/282; A61K 9/2873; A61K 9/51; A61N 5/067; A61P 35/00

(57) ABSTRACT

Embodiments of the instant disclosure relate to novel methods and compositions for treating tumors resistant to immune checkpoint inhibitors. In certain embodiments, compositions herein can have at least one nanoparticle formed of Prussian blue materials and, optionally, one or more CD137 agonists. In other embodiments, methods of treating tumors herein can include administering an effective amount of at least photothermal therapy agent in combination with at least one CD137 agonist separately or in a combination therapy/combination composition.

### CROSS REFERENCE TO RELATED APPLICATION

This application is a continuation of U.S. application Ser. No. 17/714,010, filed on Apr. 5, 2022, which claims the benefit of U.S. Provisional Application No. 63/171,452, filed on Apr. 6, 2021, each of which is hereby incorporated by reference.

### GOVERNMENTAL RIGHTS

This invention was made with government support under Grant Nos. R41CA217294 and R37CA226171 awarded by the National Institutes of Health. The government has certain rights in the invention.

### FIELD

Embodiments of the instant disclosure relate to novel methods and compositions for treating one or more tumors in a subject having immune checkpoint resistance or a subject suspected of developing immune checkpoint resistant cancers.

### BACKGROUND

Immune checkpoint inhibitors (ICIs) block interaction between tumor proteins that normally silence the immune response and their receptors on T cells. This prevents immune “exhaustion” and allows the immune response to kill the tumor. Accordingly, ICI therapy has recently become a promising therapeutic strategy with encouraging therapeutic outcomes due to their durable anti-tumor effects. Unfortunately, tumor inherent or acquired resistance to ICIs, in addition to treatment-related toxicities, have hampered the clinical utility of ICIs. As an example, about 60-70% of patients who received ICIs show no objective response to intervention. As such, there is a need in the art to develop alternative or additional cancer therapies that can compromise tumor resistance to immune checkpoint blocked therapy.

Of potential interest for treating immune checkpoint resistant cancers are thermal therapy regimens, such as photothermal therapy (PTT). Thus far, implementation of PTT has made incremental but limited progress in treating/preventing cancer. Although PTT ablates most of the tumor, the tumor is not completely eradicated. As such, the problem with PTT therapy in the current field is that it is ineffective in preventing relapse of cancerous disease. Accordingly, there is a need in the field to improve PTT beginning with the design of the photothermal therapy agents which are the underlying mechanism in successful PTT outcomes.

### SUMMARY

The present disclosure is based, at least in part, on the discovery that combining photothermal therapy, using either uncoated or functionalized Prussian blue nanoparticles (PBNPs)) with CD137 agonists is more effective for treating immune checkpoint inhibitor-resistant cancers than either therapy alone. Compositions and methods disclosed herein improves upon both singularly administered immune adjuvants/immunotherapies as well as on current photothermal therapy agents. Immune adjuvants administered directly into the bloodstream or injected into tissue are typically rapidly cleared before they can elicit a response. As demonstrated in the exemplary methods herein, using immunotherapies in combination with or loaded onto PBNPs increased clearance times and sustained bioavailability in the body of a subject. Whereas agents currently being used for photothermal therapy of cancer do not take advantage of the benefits of both endogenous and exogenous immune adjuvants, the compositions and methods disclosed herein can use specific conditions of photothermal therapy to engage immunogenic cell death, which is then augmented with immune adjuvants administered locally into the treatment (e.g., a tumor). Accordingly, the compositions and methods disclosed herein can allow for immune cell recognition of the cancer and a robust abscopal effect.

The present disclosure provides biofunctionalized nanocomposites. In certain embodiments, biofunctionalized nanocomposites disclosed herein may comprise at least a core comprising a nanoparticle formed of Prussian blue materials. In some embodiments, biofunctionalized nanocomposites disclosed herein may comprise a core comprising a nanoparticle formed of Prussian blue materials; a shell obtained by partially or completely encapsulating the Prussian blue core with a biocompatible coating; and at least one biomolecule attached to, or absorbed to, the biocompatible coating. In some embodiments, biofunctionalized nanocomposites disclosed herein may optionally comprise a shell and/or a biomolecule.

In some embodiments, biofunctionalized nanocomposites disclosed herein may have Prussian blue materials comprised of iron hexacyanoferrate (II) compounds. In some embodiments, biofunctionalized nanocomposites disclosed herein may have Prussian blue materials represented by any of the general formulas disclosed herein (e.g., formulas I, II, or III). In some aspects, biofunctionalized nanocomposites disclosed herein may have Prussian blue materials represented by general formula (II):

AxFeYIII[FeII(CN)6]z·nH2O (II)

wherein: A represents at least one of Li, Na, K, Rb, Cs, NH4 and Tl in any oxidation state and any combination thereof; X is from 0 to about 1; Y is from 0.1 to about 4; Z is from 0.1 to about 4; and N is from 1 to about 24.

In some embodiments, a biocompatible coating of the shell of biofunctionalized nanocomposites disclosed herein may comprise dextran, chitosan, silica, polyethylene glycol (PEG), avidin; a protein, a nucleic acid, a carbohydrate, a lipid, neutravidin, streptavidin, gelatin, collagen, fibronectin, albumin, a serum protein, a lysozyme, a phospholipid, a polyvinyl pyrrolidone (PVP), a polyvinyl alcohol, polyethylene glycol diacrylate, polyethylenimine (PEI), or any combination thereof. In some aspects, the biocompatible coating of the shell may comprise polyethylene glycol (PEG), a polyvinyl pyrrolidone (PVP), a polyvinyl alcohol, polyethylenimine (PEI), or any combination thereof.

In some embodiments, biomolecules attached to, or absorbed to, the biocompatible coating of biofunctionalized nanocomposites disclosed herein may comprise an antibody, a peptide, a protein, an enzyme, an amino acid, a nucleic acid, a carbohydrate, a fat, an aptamer, a small molecule, a synthetic molecule, or any combination thereof. In some aspects, biomolecules attached to, or absorbed to, the biocompatible coating of biofunctionalized nanocomposites disclosed herein may comprise an antibody. In some aspects, biomolecules attached to, or absorbed to, the biocompatible coating of biofunctionalized nanocomposites disclosed herein may comprise at least one CD137 agonist. In some aspects, a CD137 agonist attached to, or absorbed to, the biocompatible coating of biofunctionalized nanocomposites disclosed herein may be an antibody, a peptide, a protein, an enzyme, an amino acid, a nucleic acid, a carbohydrate, a fat, an aptamer, a small molecule, a synthetic molecule (e.g., synthetic ligand), or any combination thereof. In some aspects, a CD137 agonist attached to, or absorbed to, the biocompatible coating of biofunctionalized nanocomposites disclosed herein may be an antibody. In some aspects, an anti CD137 antibody agonist attached to, or absorbed to, the biocompatible coating of biofunctionalized nanocomposites disclosed herein may consist of about 500 amino acids or less, excluding zero (e.g., about 500 amino acids or less, about 450 amino acids or less, about 400 amino acids or less, about 350 amino acids or less, about 300 amino acids or less, about 250 amino acids or less, about 200 amino acids or less, about 150 amino acids or less, about 100 amino acids or less).

In some embodiments, biofunctionalized nanocomposites disclosed herein may further comprise at least one imaging agent. In some aspects, an imaging agent suitable for the biofunctionalized nanocomposites disclosed herein may be a fluorescein compound, a rhodamine compound, a xanthene compound, a cyanine compound, a naphthalene compound, a coumarin compound, an oxadiazole compound, a pyrene compound, an oxazine compound, an acridine compound, an arylmethine compound, a tetrapyrrole compound, a proprietary molecule, or any combination thereof.

In some embodiments, biofunctionalized nanocomposites disclosed herein may be stable at no less than about 80° C. (e.g., no less than about 80° C., no less than about 70° C., no less than about 60° C., no less than about 50° C., no less than about 40° C., no less than about 30°° C., no less than about 20° C.). In some embodiments, biofunctionalized nanocomposites disclosed herein may be stable for at least about 7 days (e.g., about 7, 6, 5, 4, 3, 2, 1 day(s)).

The present disclosure also provides methods of treating a subject in need thereof. In some aspects, a subject in need thereof may have or may be suspected of having a cancer. In some other aspects, a subject in need thereof may have or may be suspected of having a tumor. In some aspects, a subject in need thereof may have or may be suspected of having a cancer resistant to one or more immune checkpoint inhibitors.

In certain embodiments, methods disclosed herein may include treating at least one tumor a subject in need thereof, the method comprising administering one or more of the biofunctionalized nanocomposites disclosed herein. In some embodiments, methods disclosed herein may include treating at least one tumor a subject in need thereof, the method comprising administering one or more of the biofunctionalized nanocomposites disclosed herein and subjecting the subject to photothermal therapy. In some aspects, the photothermal therapy disclosed herein may comprise the use of a device that emits electromagnetic radiation with a wavelength that irradiates the biofunctionalized nanocomposite. In some aspects, biofunctionalized nanocomposites disclosed herein by be irradiated using a device that emits electromagnetic radiation with a wavelength between about 600 nm and about 1000 nm (e.g., about 600 nm, about 700 nm, about 800 nm, about 900 nm, about 1000 nm).

In some embodiments, methods of treatment as disclosed herein may be performed at least once a week for at least about 3 weeks (e.g., at least about 3 weeks, at least about 2 weeks, at least about 1 week).

In some embodiments, methods disclosed herein may treat a cancer that is a checkpoint inhibitor-resistant cancer. In some embodiments, methods disclosed herein may treat a subject has previously been treated with checkpoint inhibitors and is not in remission.

In some embodiments, methods disclosed herein may prevent metastasis of at least one tumor cell in a subject having checkpoint inhibitor-resistant cancer. In some embodiments, methods disclosed herein may prevent cancer recurrence of a cancer in a subject having checkpoint inhibitor-resistant cancer. In some embodiments, methods disclosed herein may generate immunological memory of a cancer in a subject having checkpoint inhibitor-resistant cancer.

In certain embodiments, a method of treating cancer in a subject having checkpoint inhibitor-resistant cancer may comprise the following steps: (a) selecting a subject having been administered a checkpoint inhibitor and a biofunctionalized nanocomposite, wherein the biofunctionalized nanocomposite comprised (i) a core comprised of a Prussian blue nanoparticle; and having been subjected to photothermal therapy; and (b) administering to the subject a CD137 agonist.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE FIGURES

The patent or application file contains at least one drawing executed in color. Copies of this patent or patent application publication with color drawing(s) will be provided by the Office upon request and payment of the necessary fec.

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-IN depict schematics and graphs illustrating PBNP-PTT generation of immunogenicity of SM1 cells in vitro. FIG. 1A shows the effect of varying thermal doses of PBNP-PTT on immunogenicity as measured by tumor cell viability, ICD correlates, cellular markers, and activation of T cells in SM1 cells (10 million cells/mL) treated with varied doses of PBNP-PTT. FIG. 1B shows cell suspension temperatures measured every minute for ten minutes using a thermal camera. FIG. 1C shows thermal dose (ΣCEM43) values. FIGS. 1D-1L show cell viability (FIG. 1D), intracellular ATP (FIG. 1E), calreticulin (FIG. 1F), intracellular HMGB1 (FIG. 1G), CD80 (FIG. 1H), CD86 (FIG. 1I), MHC1 (FIG. 1J), CD137L (FIG. 1K), and MART-1 (FIG. 1L) visualized on SM1 cells 24 hours after treatment. Values indicate MFI. FIG. 1M shows SM1 cells that were left untreated (CTRL) or treated in vitro with Vehicle (PBS), PBNPs, or PBNP-PTT, and then co-cultured with ex vivo T cells isolated from spleens of naïve mice at an E:T ratio of 5:1. After 48 hours, CD69 expression was measured on live T cells. FIG. 1N shows PBNP-PTT-treated SM1 cells expressing an APC-like phenotype. Values represent means±standard deviation (SD), n=3/group; ns: not significant, **p<0.01, ***p<0.001, **** p<0.0001.

FIGS. 2A-2H depict graphs illustrating PBNP-PTT effectively treated local but not distal SM1 melanoma in vivo. FIGS. 2A-2B and 2G show the effect of PBNP-PTT on tumor growth and survival. Tumor-bearing mice were left untreated (CTRL) or treated with PBNP-PTT where FIG. 2A shows tumor growth observed in the treatment groups and FIG. 2B shows survival of tumor-bearing animals post-treatment and rechallenge. FIG. 2G shows tumor temperatures attained during PBNP-PTT as measured by a thermal camera. Inset: thermal dose log (ΣCEM43) administered during PBNP-PTT. n=4/group, **p<0.01. FIGS. 2C-2F and 2H show the effect of PBNP-PTT on tumor growth and survival in a two tumor model. Tumor-bearing mice were left untreated (CTRL) or treated with PBNP-PTT or PBNP-PTT+aPD-1. When used, PBNP-PTT was administered to only one (“primary”) tumor, and the other (“secondary”) tumor was left untreated. Tumor growth observed in mice treated with CTRL (FIG. 2D), PBNP-PTT (FIG. 2D), and PBNP-PTT+aPD-1 (FIG. 2E). Solid lines represent the primary (treated) tumor, dashed lines represent the secondary (untreated) tumor. FIG. 2H shows tumor temperatures attained during PBNP-PTT as measured by a thermal camera. Inset: thermal dose log (ΣCEM43) administered during PBNP-PTT. Value represents mean±st. dev. FIG. 2F shows the survival of tumor-bearing animals post-treatment. n=4/group; ** p <0.01, ns: not significant.

FIGS. 3A-3I depict schematics and graphs illustrating that PBNP-PTT+aCD137 improved the survival of metachronous tumor-bearing mice through an abscopal effect. FIG. 3A shows a schematic overview of the study where two SM1 tumors were inoculated into opposite sides of C57BL/6 mice four days apart and secondary tumors were left untreated. FIGS. 3B-3F show tumor growth observed in mice treated with CTRL (FIG. 3B), aCD137 (FIG. 3C), PBNP-PTT (FIG. 3D), and PBNP-PTT+aCD137 (FIG. 3E), with tumor growth curves superimposed for comparison (FIG. 3F). Each line represents an individual mouse; solid lines represent the primary tumors and dashed lines represent the secondary tumors. FIG. 3G shows survival of tumor-bearing animals post-treatment. n=4-5/group; *p<0.05, **p<0.01 compared with CTRL and aCD137. FIG. 3H shows tumor temperatures attained during PBNP-PTT as measured by a thermal camera. FIG. 3I shows thermal dose log (ΣCEM43) administered during PBNP-PTT. Values represent mean±st. dev.

FIGS. 4A-4G depict schematics and graphs illustrating that PBNP-PTT+aCD137 generated an abscopal antitumor effect via infiltrating cytotoxic T cells. FIG. 4A shows a schematic overview of the study wherein two SM1 tumors were inoculated into opposite sides of C57BL/6 mice simultaneously and secondary tumors were left untreated. FIGS. 4B-4C show primary (FIG. 4B) and secondary (FIG. 4C) tumor growth. FIGS. 4D-4E show secondary tumor-infiltrating CD8+ T cells (FIG. 4D) and CD4+ T cells (FIG. 4E) after 14 days. Values represent means±SD, n=5/group; ns: not significant, *p<0.05, **p<0.01. FIG. 4F shows tumor temperatures attained during PBNP-PTT as measured by a thermal camera. FIG. 4G shows thermal dose log (ΣCEM43) administered during PBNP-PTT. Values represent mean±st. dev.

FIGS. 5A-5M depict graphs illustrating that PBNP-PTT+aCD137 enhanced DC activation in lymph nodes and generated systemic T cell activation and memory. Synchronous SM1 tumors were established and treated as described. FIGS. 5A-5C show lymph nodes that were harvested and analyzed for CD11c (FIG. 5A), CD80 (FIG. 5B), and CD86 (FIG. 5C) expression. FIGS. 5D-5M show spleens that were harvested and analyzed for: CD4 (FIGS. 5D-5F); CD8 (FIGS. 5G-51); CD25 (FIGS. 5E and 5H); CD69 (FIGS. 5F and 5I); and CD62 and CD44 (FIGS. 5J-5M) expression levels. Values represent means±SD, n=5/group; ns: not significant, *p<0.05, **p<0.01, ***p<0.001, and ****p<0.0001.

FIGS. 6A-6F depict graphs illustrating that PBNP-PTT+aCD137 generated an increase in serum cytokines/chemokines associated with T cell activation. Synchronous SM1 tumors were established and treated as previously described. On Day 14, blood was harvested and serum was analyzed for TNFα (FIG. 6A), IL-5 (FIG. 6B), IL-10 (FIG. 6C), Fas ligand (FIG. 6D), MIP-1a (FIG. 6E), and RANTES (FIG. 6F) expression. Normalized values in the negative range are listed as “0” pg/mL. Values represent mean±SD, n=5/group; ns: not significant, *p<0.05, **p<0.01, ***p<0.001, ****p<0.0001.

FIGS. 7A-7I depict schematics and graphs illustrating that PBNP-PTT+aCD137 improved the survival of single tumor-bearing mice and generated immunological memory. FIG. 7A shows a schematic overview of the study where SM1 tumor-bearing mice were left untreated (CTRL) or treated with PBNP-PTT, aCD137, or PBNP-PTT+aCD137. Long-term surviving mice were rechallenged with SM1 cells. FIGS. 7B-7F show tumor growth observed in the CTRL (FIG. 7B), aCD137 (FIG. 7C), PBNP-PTT (FIG. 7D), PBNP-PTT+aCD 137 (FIG. 7E), and all (FIG. 7F) treatment groups. FIG. 7G shows survival of tumor-bearing animals post-treatment. n=5/group; *p<0.05, **p<0.01. The values given in red represent number of surviving animals before and post-rechallenge. FIG. 7H shows tumor temperatures attained during PBNP-PTT as measured by a thermal camera. FIG. 7I shows thermal dose log (ΣCEM43) administered during PBNP-PTT. Values represent means±st. dev.

FIG. 8 shows a schematic of a mechanism of action for PBNP-PTT+aCD137, illustrating the critical involvement of activated DCs and CD4+and CD8+T cells in generating abscopal efficacy against melanoma.

FIGS. 9A-9L depict schematics and graphs illustrating the synthesis and characterization of Prussian blue nanoparticles (PBNPs). FIG. 9A shows PBNPs prepared by a co-precipitation method using FeCl₃·6H₂O and K₄[Fe(CN)₆]·3H₂O. The co-precipitated nanoparticles were washed to eliminate excess unreacted salts. FIGS. 9B-9C show dynamic light scattering and zeta potential graphs depicting monodispersed PBNPs of size 50 nm that were negatively charged (<−30 mV) and stable over a time period of 6 months. FIG. 9D shows a UV-Vis-NIR spectrum of PBNPs depicting a wide absorption peak ranging from 600 to >800 nm, making them suitable for excitation using an 808 NIR laser for photothermal therapy. FIGS. 9E-9F show that heating of PBNPs by laser illumination was laser power dependent, as shown by (FIG. 9E) increasing temperature and (FIG. 9F) thermal dose (ΣCEM43) with laser powers ranging from 0.2 W to 2 W. FIG. 9G shows that 2 W PBNP-PTT identically heated PBNPs over four cycles of heating in water (blue), but cyclic heating in complete cell culture media with 10% FBS (purple) was less effective after the first cycle of PBNP-PTT. FIG. 9H shows hydrodynamic diameter distribution of PBNPs at pH 7 or pH 6 in complete cell culture media with 10% FBS at room temperature (RT) over seven days, as compared to PBNPs in water (black). FIG. 9I shows hydrodynamic diameter distribution of PBNPs at pH 7 or pH 6 in complete cell culture media with 10% FBS at 37° C. over seven days, as compared to PBNPs in water (black). The peak representing smaller sizes (˜10 nm) likely reflects media components (e.g., proteins in serum), and the increase in hydrodynamic diameter may be caused by a protein corona formed on the surface of PBNPs upon dispersion in serum-containing media. FIG. 9J shows a representative hydrodynamic diameter distribution of PBNP-free complete cell culture media with 10% FBS (pH 6 at RT on Day 1), illustrating a peak ˜10 nm. FIG. 9K shows zeta potential of PBNPs that was measured in complete cell culture media with 10% FBS at room temperature (RT) or 37° C. (37C) at pH 7 or 6 over seven days. FIG. 9L shows transmission electron microscopy (TEM) images illustrating the morphology of PBNPs. Scale bar=100 nm (left) and 200 nm (right).

FIGS. 10A-10E depict images and graphs illustrating physical characteristics of PBNP-PTT in vitro. FIG. 10A shows thermal images of SM1 murine melanoma cells treated with 0.15 mg mL-1 PBNPs and exposed to 10-minute laser illumination. Each panel displays the thermal distribution (heat map) that indicated the maximum temperature reached while performing in vitro PBNP-PTT using different laser powers (0.75 W-2 W). FIG. 10B shows bright-field images of SM1 cells 24 hours post-PBNP-PTT depicting increased dead and floating cells (loss of confluence) with increased temperature/laser power. Bright-field images were taken at 20× magnification using Leica DMi1 inverted light microscope. FIG. 10C shows PBNP size distribution (hydrodynamic diameter) that was measured after 10 minutes PBNP-PTT in media at varied thermal doses. FIG. 10D shows PBNP surface charge (zeta potential) measured after 10 minutes PBNP-PTT in media at varied thermal doses. FIG. 10E shows, as a measure of degradation, PBNP concentration measured after 10 minutes PBNP-PTT at varied thermal doses. A thermal dose-dependent degradation of PBNPs was observed.

FIGS. 11A-11D depict images and graphs illustrating an analysis of SM1 cells for immunogenic cell death (ICD) markers. Scatter plots showing (FIG. 11A) an increase in surface calreticulin and (FIG. 11B) a decrease in intracellular levels of HMGB1 for SM1 cells treated with higher laser power, indicating the generation of ICD with increased laser power and thermal dose. FIG. 11C shows an analysis of extracellular ATP in the supernatant of cell culture media after 10 minutes PBNP-PTT at varied thermal doses. Extracellular ATP was measured by the RealTime-Glo Extracellular ATP Assay (Promega) following the manufacturer's protocol. FIG. 11D shows an analysis of extracellular HMGB1 in the supernatant of cell culture media after 10 minutes PBNP-PTT at varied thermal doses. Extracellular HMGB1 was measured by the HMGB1 Detection ELISA Kit (Chondrex) following the manufacturer's protocol.

FIGS. 12A-12E depict images and graphs illustrating flow cytometry analysis of SM1 cells for expression of immunostimulatory molecules after PBNP-PTT. FIGS. 12A-12E show scatter plots depicting increases in molecules associated with T cell stimulation, (FIG. 12A) CD80, (FIG. 12B) CD86, (FIG. 12C) MHC-I, (FIG. 12D) CD137L, and (FIG. 12E) Melanoma Antigen Recognized by T cells (MART-1) expressed on the surface of surviving SM1 cells treated with PBNP-PTT at varying laser powers, suggesting increased immunogenicity with increased laser power and thermal dose.

FIGS. 13A-13K depict images and graphs illustrating an analysis of WM9 human melanoma cells for expression of ICD markers and immunostimulatory molecules after PBNP-PTT. FIG. 13A shows a temperature curve showing consistent increase in temperature in a laser-power dependent manner, with the highest temperature of 85.5° C. reached with 2 W PBNP-PTT. FIG. 13B shows a CEM43 graph indicating an increase in thermal dose in a laser-power dependent manner. FIG. 13C shows a viability assay by flow cytometry depicting WM9 cell death in a thermal-dose dependent manner. FIGS. 13D-13F show an analysis of molecules associated with ICD (ATP, calreticulin, and HMGB1) depicting (FIG. 13D) increased ATP release (indicated by lower intracellular ATP), (FIG. 13E) increased surface calreticulin, and (FIG. 13F) decreased intracellular HMGB1 in a thermal dose-dependent manner. FIGS. 13G-13K show a flow cytometry analysis demonstrating an increase in molecules associated with T cell stimulation, (FIG. 13G) CD80, (FIG. 13H) CD86, (FIG. 13I) HLA-class I (HLA-A,B,C), (FIG. 13J) CD137L, and (FIG. 13K) MART-1 expressed on surface of surviving WM9 human melanoma cells treated with PBNP-PTT at varying laser powers, suggesting increased immunogenicity with increased laser power and thermal dose, consistent with the SM1 murine melanoma model.

FIGS. 14A-14K depict images and graphs illustrating an analysis of WM793 human melanoma cells for expression of ICD and immunostimulatory molecules after PBNP-PTT. FIG. 14A shows a temperature curve demonstrating a consistent increase in temperature in a laser-power dependent manner with highest temperature of 83.2° C. reached with 2 W PBNP-PTT. FIG. 14B shows a CEM43 graph indicating an increase in thermal dose in a laser-power dependent manner. FIG. 14C shows a viability assay by flow cytometry demonstrating WM793 cell death in a thermal-dose dependent manner. FIGS. 14D-14F show an analysis of molecules associated with ICD (ATP, calreticulin, and HMGB1) demonstrating (FIG. 14D) increased ATP release (indicated by lower intracellular ATP), (FIG. 14E) increased surface calreticulin and (FIG. 14F) decreased intracellular HMGB1 in a thermal dose-dependent manner. FIGS. 14G-14K show a flow cytometry analysis demonstrating an increase in molecules associated with T cell stimulation, (FIG. 14G) CD80, (FIG. 14H) CD86, (FIG. 14I) HLA-class I (HLA-A,B,C), (FIG. 14J) CD137L, and (FIG. 14K) MART-1 expressed on surface of surviving WM793 human melanoma cells treated with PBNP-PTT at varying laser powers, suggesting increased immunogenicity with increased laser power and thermal dose, consistent with the SM1 murine melanoma model.

FIGS. 15A-15E depict graphs illustrating ex vivo T cell activation by PBNP-PTT-treated SM1 cells. At an E:T ratio of 5:1, T cells co-cultured for 24 hours with SM1 melanoma cells pre-treated with PBNP-PTT showed (FIG. 15A) small but statistically significant changes in CD25 expression at several laser powers studied (0.75 W-2 W), and (FIG. 15B) no changes in CD69 expression. FIGS. 15C-15D show that, at an E:T ratio of 10:1, T cells co-cultured for 24 hours with SM1 melanoma cells pre-treated with PBNP-PTT demonstrated (FIG. 15C) small but statistically significant changes in CD25 at some conditions studied, and (FIG. 15D) no changes in CD69 expression. FIG. 15E shows that, at an E:T ratio of 10:1, T cells co-cultured for 48 hours with SM1 melanoma cells pre-treated with PBNP-PTT demonstrated small but statistically significant changes in CD25 at many conditions studied. Statistical analysis: One-way ANOVA using Tukey's multiple comparison test. ns: not significant, *p<0.05, **p<0.01, ***p<0.001, ****p<0.0001.

FIGS. 16A-16E depict graphs illustrating that ICI immunotherapy was ineffective to treat SM1 melanoma in vivo. FIGS. 16A-16B show that, in single SM1 tumor-bearing mice left untreated (CTRL) or treated with i.p. aPD-1, there was no difference in (FIG. 16A) tumor growth or (FIG. 16B) survival between the groups. FIGS. 16C-16D show that, in two synchronous SM1 tumor-bearing mice left untreated or treated with i.p. aPD-1, there was no difference in (FIG. 16C) tumor growth or (FIG. 16D) survival between the groups. FIG. 16E shows that in single SM1 tumor-bearing mice treated with an isotype control and different concentrations (5, 10 and 15 mg mL⁻¹) of aCTLA-4, there were no changes in average tumor growth across the groups. Statistical analysis on survival graph: Log-rank (Mantel-cox) test. ns: not significant; n=4/group.

FIGS. 17A-17C depict graphs illustrating a flow cytometry analysis of melanoma cells for expression of PD-L1 after PBNP-PTT. PD-L1 levels were found to be unchanged or decreased from basal expression at all PBNP-PTT conditions in both the mouse SM1 melanoma cells (FIG. 17A) and human melanoma cell lines, (FIG. 17B) WM9 and (FIG. 17C) WM793. These results suggested that aPD-1 therapy may not improve the efficacy of PBNP-PTT.

FIGS. 18A-18D depict images and graphs illustrating a representative in vivo PBNP-PTT setup. FIGS. 18A-18B show representative digital photographs depicting the preparation of C57BL/6 mice carrying two tumors, where (FIG. 18A) one tumor (primary) was injected intratumorally with 50 μL of 1 mg mL⁻¹ PBNP and the second tumor (secondary) was left untreated, followed by (FIG. 18B) laser illumination of the PBNP-injected primary tumor with an 808 NIR laser for 10 minutes. The entire procedure was carried out under general gas anesthesia and the eyes of the animals were covered to protect from damage. FIGS. 18C-18D show representative IR images depicting the thermal distribution of the tumor (marked by + symbol), indicating the maximum temperature reached during PBNP-PTT. All animals (10 mice shown here) were treated with PBNP-PTT. Post-treatment, the animals were randomly separated into two groups, (FIG. 18C) PBNP-PTT alone and (FIG. 18D) PBNP-PTT+aCD137. The latter group received six i.p. doses of aCD137 over the course of two weeks following PBNP-PTT treatment.

FIGS. 19A-19B depict images illustrating an analysis of liver metastasis. FIG. 19A shows a representative histology image illustrating SM1 tumor metastasis in the liver (indicated by red arrows). Metastasis was defined as a group of 10 or more tumor cells. FIG. 19B shows a table listing the percentage of animals harboring liver metastases in the four treatment groups.

FIGS. 20A-20B depict images illustrating a flow cytometry analysis of secondary untreated tumors for T cells. FIG. 20A shows representative scatter plots demonstrating no change in the levels of CD4+ T cells within secondary untreated tumors of CTRL animals and animals treated with aCD137 alone, PBNP-PTT, or PBNP-PTT+aCD137. FIG. 20B shows representative scatter plots demonstrating a significant increase in CD8+ cytotoxic T cells within secondary tumors in groups treated with aCD137 (aCD137 alone and PBNP-PTT+aCD137) compared to CTRL and PBNP-PTT treatment groups. The average amount of CD8+ T cells was higher in mice treated with PBNP-PTT+aCD137 (i.e., 31.4% depicted here) compared to aCD137 alone (i.e., 23.8% depicted here).

FIGS. 21A-21C depict images illustrating a flow cytometry analysis of lymph nodes for migration and maturation status of dendritic cells (DCs). FIG. 21A shows representative scatter plots demonstrating increased CD11c+DCs in lymph nodes of animals treated with PBNP-PTT+aCD137 compared to all other treatment groups. FIGS. 21B-21C show representative scatter plots analyzing DC maturation markers CD80+ and CD86+ expressed on CD11c+DCs. There was (FIG. 21B) a significant increase in CD11c+/CD80+ DCs in mice treated with PBNP-PTT+aCD137 compared to all other groups, but (FIG. 21C) no change in CD11c+/CD86+DCs in lymph nodes of animals treated with PBNP-PTT+aCD137 compared to CTRL.

FIGS. 22A-22C depict images illustrating a flow cytometry analysis of CD4+ T cell activation status in the spleen. FIG. 22A shows representative scatter plots demonstrating a mild decrease in CD4+ T cells in the spleens of mice treated with PBNP-PTT+aCD137 compared to CTRL and PBNP-PTT-treated mice. FIG. 22B shows representative scatter plots demonstrating a significant increase in the T cell activation marker CD25 on CD4+ T cells in mice treated with PBNP-PTT+aCD137 compared to CTRL. FIG. 22C shows representative scatter plots demonstrating a significant increase in the T cell activation marker CD69 on CD4+ T cells in mice treated with PBNP-PTT+aCD137 compared to all other treatment groups. This illustrates that despite the decrease in the CD4+ T cell population in mice treated with the combination therapy, the splenic CD4+ T cells were more activated compared to CTRL and monotherapies.

FIGS. 23A-23C depict images illustrating a flow cytometry analysis of CD8+ T cell activation status in the spleen. FIG. 23A shows representative scatter plots demonstrating a significant increase in CD8+ T cells in the spleens of mice treated with PBNP-PTT+aCD137compared to all other treatment groups. FIG. 23B shows representative scatter plots demonstrating a significant increase in the T cell activation marker CD25 on CD8+ T cells in the PBNP-PTT+aCD137 treatment group compared to CTRL and PBNP-PTT alone. FIG. 23C shows representative scatter plots demonstrating a significant increase in activation marker CD69 on CD8+ T cells for groups treated with aCD137 and PBNP-PTT+aCD137 compared to CTRL.

FIGS. 24A-24B depict graphs illustrating a flow cytometry analysis of exhaustion status of T cells in spleen. FIGS. 24A-24B show violin plots illustrating no change in the T cell exhaustion marker, PD-1, on cither (FIG. 24A) CD4+ or (FIG. 24B) CD8+ splenic T cells across all treatment groups. Statistical analysis was done using one-way ANOVA, Tukey's multiple comparison test.

FIGS. 25A-25B depict images and graphs illustrating a flow cytometry analysis of naïve vs memory T cells in spleen. FIGS. 25A-25B show representative scatter plots showing significant decrease in CD62L+/CD44− naïve (FIG. 25A) CD4+ and (FIG. 25B) CD8+ T cells and significant increase in CD44+/CD62L− memory (FIG. 25A) CD4+ and (FIG. 25B) CD8+ T cells in spleen of animals treated with aCD137 or PBNP-PTT+aCD137 compared to CTRL and PBNP-PTT.

FIGS. 26A-26B depict graphs illustrating a tumor rechallenge compared to naïve age-matched animals. Long-term surviving animals after single tumor challenge were rechallenged with SM1 tumors on day 66 post-treatment initiation. FIG. 26A shows a tumor growth curve demonstrating complete tumor rejection in ⅔ surviving animals previously treated with PBNP-PTT+aCD137, suggesting long-term immunological memory, compared to naïve ( 0/5 rejection, all challenged animals grew tumors) and PBNP-PTT ( 0/2 rejection, all rechallenged animals grew tumors). FIG. 26B shows a Kaplan-Meier plot demonstrating 66% long-term tumor-free survival for PBNP-PTT+aCD137-treated animals up to 110 days post-rechallenge, compared to the naïve and PBNP-PTT group. Statistical analysis was done using Log-rank (Mantel-Cox) test. *p<0.05.

FIGS. 27A-27F depict graphs and images illustrating an analysis of acute hepatotoxicity. FIGS. 27A-27D show representative histology sections demonstrating no inflammation in (FIG. 27A) CTRL and (FIG. 27B) PBNP-PTT-treated animals, and clusters of inflammatory cells (indicated by yellow arrows) near hepatic blood vessels in both (FIG. 27C) aCD137 and (FIG. 27D) PBNP-PTT+aCD137-treated animals. FIGS. 27E-27F shows scores for inflammation in the lobules, central vein, and portal tracts of livers. Overall inflammation, as quantified across the three examined locations, was also measured, showing increased inflammation in aCD137-containing treatment groups.

FIGS. 28A-28C depict graphs illustrating an analysis of chronic hepatotoxicity on long-term surviving PBNP-PTT+aCD137-treated mice. FIG. 28A shows inflammation scores for livers from long-term surviving PBNP-PTT+aCD137-treated mice (n=2; green) and age-matched naïve mice (n=3; gray) in the lobules, central vein, and portal tracts. Overall inflammation, as quantified across the three examined locations, was also measured. Treated mice showed significantly higher portal tract inflammation (scored as mild inflammation) than controls, but no significant difference in overall liver hepatic inflammation. FIGS. 28B-28C shows sera analysis of (FIG. 28B) alanine aminotransferase (ALT) and (FIG. 28C) aspartate aminotransferase (AST), showing no significant difference between longterm surviving PBNP-PTT+aCD137-treated animals (226 days post-treatment) (n=2) compared to age-matched naïve animals (CTRL, n=3). Significance was calculated using an unpaired t-test and the respective estimation plots arc provided alongside the average ALT/AST levels. Each analysis had 3 technical replicates per animal studied.

FIGS. 29A-29H depict graphs illustrating characterization of aCD137-PBNP PTT and booster nanoparticles. FIGS. 29A-29C show dynamic light scattering of control and aCD137-PBNP PTT nanoparticles. FIGS. 29D-29F show dynamic light scattering of control and aCD137-PBNP booster nanoparticles. FIG. 29G shows a zeta potential graph for control and aCD137-PBNP PTT nanoparticles. FIG. 29H shows a zeta potential graph for control and aCD137-PBNP PTT nanoparticles.

FIGS. 30A-30E depict graphs illustrating tumor volume in mice treated with aCD137 (FIG. 30B), aCD137-PBNP (FIG. 30C), PBNP PTT (FIG. 30D), aCD137-PBNP PTT +2 booster nanoparticles (aCD137-PBNPs administered at the initial site of tumor without PTT) (FIG. 30E) or untreated (FIG. 30A).

FIGS. 31A-31B depict graphs illustrating optimum mass ratio for αCD137-PBNP synthesis. FIG. 31A shows coating efficiencies determined from BCA Assay using mass of αCD-137 below a 2:1 PBNP to αCD137 ratio. FIG. 31B shows coating efficiencies determined from BCA Assay using mass of αCD137 above 2:1 PBNP to αCD137 ratio.

FIGS. 32A-32G depict graphs illustrating the characterization of rising amounts of αCD-137 in 250 μg PBNP. FIG. 32A shows the zeta potential for the PBNPs after sitting for 0, 1, 2, and 3 days after synthesis. FIGS. 32B-32G shows the hydrodynamic diameters for the PBNPs after sitting for 0 and 2 days after synthesis.

FIGS. 33A-33F depict graphs illustrating the characterization of decreasing amounts of αCD-137 in 250 μg PBNP. FIG. 33A shows the zeta potential for the PBNPs after sitting for 0, 1, and 2 days after synthesis. FIGS. 32B-32F shows the hydrodynamic diameters for the PBNPs after sitting for 0 and 2 days after synthesis.

### Definitions

Terms, unless defined herein, have meanings as commonly understood by a person of ordinary skill in the art relevant to certain embodiments disclosed herein or as applicable.

As used herein “about” unless otherwise indicated, applies to all numbers expressing quantities of agents and/or compounds, properties such as molecular weights, reaction conditions, and as disclosed herein are contemplated as being modified in all instances by this term. Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals.

As used herein, the terms “therapeutically effective amount” or “therapeutically effective dose,” or similar terms used herein are intended to mean an amount of an agent (e.g., biofunctionalized nanocomposite) that will elicit the desired biological or medical response (e.g., an improvement in one or more symptoms of a cancer).

The terms “treat,” “treating,” and “treatment,” as used herein, refer to therapeutic or preventative measures described herein. The methods of “treatment” employ administration to a subject, in need of such treatment, a human antibody of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment. Accordingly, “treat,” “treating,” or “treatment” can refer to reversing, ameliorating, or inhibiting onset or inhibiting progression of a health condition or disease or a symptom of the health condition or disease.

As used herein, “marker” can refer to any molecule that can be measured or detected, for example. For example, a marker can include, without limitations, a nucleic acid, such as, a transcript of a gene, a polypeptide product of a gene, a glycoprotein, a carbohydrate, a glycolipid, a lipid, a lipoprotein, a carbohydrate, and/or a small molecule. As used herein, “expression” and grammatical equivalents thereof, in the context of a marker, can refer to production of the marker as well as level or amount of the marker.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the present disclosure relate to novel compositions and methods for generating ex vivo expanded immune cells specific to at least one target cancer. In certain embodiments, ex vivo expanded immune cells generated as disclosed herein may be and/or may comprise an immunotherapy composition. In certain embodiments, ex vivo expanded immune cells generated as disclosed herein may be a pharmaceutical composition. In certain embodiments, ex vivo expanded immune cells generated as disclosed herein may be used to treat, prevent, and/or attenuate a cancer.

### (I) Compositions

Aspects of the present disclosure include compositions encompassing a biofunctionalized nanocomposite. A composition disclosed herein may encompass a nanoparticle formed of Prussian blue materials, a biocompatible coating, and a biomolecule. A composition disclosed herein may further comprise one or more imaging agents. A composition disclosed herein may be a photothermal therapy agent.

### (a) Prussian Blue Materials

In certain embodiments, compositions disclosed herein comprise a nanoparticle formed of at least one or more Prussian blue materials. As used herein, “Prussian blue materials”, “Prussian blue” and “Prussian blue compounds” are used interchangeably. Unless indicated otherwise, the symbols used to represent the elements of which the Prussian blue materials and/or analogs thereof of the present disclosure are comprised are the symbols used in the periodic table of elements to represent the chemical elements (for example, “Fe” represents iron, etc.).

In certain embodiments, compositions disclosed herein may comprise doped Prussian blue compounds. In various aspects, compositions disclosed herein may comprise Prussian blue materials represented by general formula (I):

AₓB_{y}M₄[M′(CN)₆]_{z}·nH₂O (I),

which is coated with a biocompatible shell onto which targeting, imaging and/or therapeutic agents are attached. In the compounds of general formula (I), A represents at least one of VO₂, Ca, V, Cr, Mn, Fe, Co, Ni, Cu, Zn, Ga, Sr, Zr, Nb, Li, Na, K, Rb, Cs, Fr, TI, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof; B represents at least one of VO₂, Ca, V, Cr, Mn, Fe, Co, Ni, Cu, Zn, Ga, Sr, Zr, Nb, Li, Na, K, Rb, Cs, Fr, TI, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof; M represents at least one of VO₂, Ca, V, Cr, Mn, Fe, Co, Ni, Cu, Zn, Ga, Sr, Zr, Nb, Li, Na, K, Rb, Cs, Fr, TI, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof; M′ represents at least one of VO₂, Ca, V, Cr, Mn, Fe, Co, Ni, Cu, Zn, Ga, Sr, Zr, Nb, Li, Na, K, Rb, Cs, Fr, TI, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof; x is from 0.1 to about 1; y is from 0.1 to about 1; z is from 0.1 to about 4; and n is from 0.1 to about 24.

In some embodiments, A represents at least one of VO₂, Ca, V, Cr, Mn, Fe, Co, Ni, Cu, Zn, Ga, Sr, Zr, Nb, Li, Na, K, Rb, Cs, Fr, TI, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof. In the further preferred embodiments, A represents at least one of Li, Na, K, Rb, Cs, and Fr, in any oxidation state and in any combination thereof. In other preferred embodiments, A represents Li, Na, K, Rb, in any oxidation state and in any combination thereof. In other preferred embodiments, A represents a mixture of K and other elements represented by A, where the molar ratio of K in the mixtures is at least 0.9, preferably, at least 0.95, most preferably at least 0.99. In the most preferred embodiments, A only represents K.

In some embodiments, B represents at least one of Cr, Mn, Fe, Eu, Gd, and Tb, in any oxidation state and in any combination thereof. In other preferred embodiments, B represents a mixture of Mn, Gd, and other elements represented by A, where the molar ratio of the combination of Mn and Gd in the mixtures is at least 0.9, preferably, at least 0.95, most preferably at least 0.99. In the most preferred embodiments, A represents a mixture of only Mn and Gd, in any oxidation state and in any combination thereof.

In some embodiments, M represents at least one of Fe, Co, and Ni, in any oxidation state and in any combination thereof. In the most preferred embodiments, M represents only Fc. In still other embodiments, M′ represents at least one of Fe, Co, and Ni, in any oxidation state and in any combination thereof. In yet other embodiments, M′ represents only Fe. In preferred embodiments, each of M and M′, simultaneously, represents only Fe, in any oxidation state thereof.

As used herein, the term “in any combination thereof” for A, B, M, and M′ means that at least two of the elements that are represented by A, B, M, and M′ can be present in any molar ratios so long as the sum total is equal to the value for x, y, and z, and, in the case of M, the elements can be present in any molar ratios so long as the total amount of the M elements is equal to 4. Preferably, x in general formula (I) is from 0.2 to 0.9, more preferably from 0.3 to 0.8, more preferably still from 0.4 to 0.7, and most preferably from 0.5 to 0.6. Preferably, y in general formula (I) is from 0.2 to 0.9, more preferably from 0.3 to 0.8, more preferably still from 0.4 to 0.7, and most preferably from 0.5 to 0.6. Preferably, z in general formula (I) is from 0.2 to 3.5, more preferably from 0.3 to 0.8, more preferably still from 0.4 to 0.7, and most preferably from 0.5 to 0.6. All real numbers within the ranges for x, y, z, and n are included.

In some embodiments, a particularly preferred species of the Prussian blue compound represented by general formula (I) are K_{0.53}Gd_{0.89}Fe^{III} ₄[Fe^{II}(CN)₆]_{3.8}·1.2H₂O and K_{0.6}Mn_{0.7}Fe_{III 4}[Fe^{II}(CN)₆]_{3.5}·3H₂O.

In some embodiments, compositions disclosed herein may comprise Prussian blue materials that belong to the class of iron hexacyanoferrate (II). In certain embodiments, compositions disclosed herein may comprise Prussian blue materials represented by general formula (II):

AxFeYIII[FeII(CN)6]z·nH₂O (II)

wherein A represents at least one of Li, Na, K, Rb, Cs, NH4 and Tl in any oxidation state and any combination thereof; X is from 0 to about 1; Y is from 0.1 to about 4; Z is from 0.1 to about 4; and N is from 1 to about 24.

In some embodiments, compositions disclosed herein may comprise Prussian blue salts represented by general formula (III):

A₄ₓFe₄₋ₓ^{III}[Fe^{II}(CN)₆]₃₊ₓ·nH₂O (III)

where A is an alkali metal such as lithium (Li+), sodium (Na⁺), Potassium (K⁺), Rubidium (Rb⁺), Cesium (Cs⁺), or it can be Ammonium (NH⁴⁺) or Thallium (Tl⁺). The value x can be any number, e.g. a fraction, from 0≤x≤1, e.g. 0.1 and n is about 1 to about 24, and preferably is from about 14 to about 16.

In some embodiments, compositions disclosed herein may comprise soluble Prussian blue materials, insoluble Prussian blue materials, or a combination thereof. In some aspects, insoluble Prussian blue materials may be characterized by coordinating water molecules therein.

In certain embodiments, compositions disclosed herein may comprise one or more metal isotopes doped to the Prussian blue materials described herein. In some aspects, a metal isotope may be Li, Na, K, Rb, Cs, Fr, Ga, In, Tl, Ca, Sc, V, Cr, Mn, Fe, Co, Ni, Cu, Zn, Sr, Y, Zr, Nb, Mo, Ru, Rh, Pd, Ag, Cd, Ba, La, Sm, Eu, Gd, Tb, Dy, Ho, Lu, Hf, Ta, W, Re, Os, Ir, Pt, Au, Hg, Pb, Bi or a combination thereof. In some embodiments, the metal isotope may be Cs, Ga, TI, In, V, Cr, Mn, Fc, Co, Ni, Cu, Mo, Ru, Ag, W, Pt, Au, Hg, Eu, Gd, or any combination thereof.

In some embodiments, a metal isotope suitable for use in the compositions disclosed herein may be present in any sufficient oxidation state theoretically possible. In some aspects, a metal isotope may be Li(I), Na(I), K(I), Cs(I), Fr(I), Ga(III), In(III), Tl(I), Tl(III), Ca(II), Sc(III), V(III), V(IV), Cr(II), Cr(III), Mn(II), Mn(IV), Fc(II), Fc(III), Co(II), Co(III), Ni(II), Ni(III), Cu(I), Cu(II), Zn(II), Sr(II), Y(III), Zr(IV), Nb(IV), Nb(V), Mo(IV), Mo(V), Ru(III), Ru(IV), Rh(II), Rh(III), Rh(IV), Pd(II), Pd(IV), Ag(I), Cd(II), Ba(II), La(III), Sm(II), Sm(III), Eu(II), Eu(III), Gd(III), Tb(III), Tb(IV), Dy(III), Ho(III), Lu(III), Hf(IV), Ta(V), W(IV), W(V), Re(I), Os(IV), Ir(II), Ir(III), Pt(II), Pt(IV), Au(I), Au(III), Hg(I), Hg(II), Pb(II), Pb(IV), Bi(III), or any combination thereof.

In certain embodiments, a metal isotope suitable for use in the compositions disclosed herein may be linked to Prussian blue materials in a chemical or physical route. As a non-limiting example of chemical linkage, a metal isotope is bound by covalent bond, whereas the metal isotope replaces the Fe atom in the complex structure of a Prussian blue compound. In some aspects, Prussian blue materials may be represented by general formula (I):

AₓB_{y}M₄[M′(CN)₆]_{z}·nH₂O (I),

wherein M and M′ denote the same or different and independently from each other Cu-61, Cu-64, Cu-67, Zn-62, Zn-69m, Zn-69, Pb-206, Pb-207, Pb-208, Pb-209, Pb-210, Pb-211, Pb-212, Pb-213, Pb-214, Ag-105, Ag-106, Ag-112, Ag-113, Pt-186, Pt-187, Pt-188, Pt-190, Pt-191, Pt-197, La-131, La-132, La-133, La-135, La-140, La-141, La-142, Eu-150m, Eu-152m, Eu-158, Eu-145, Eu-146 and Eu-147, especially Cu-61, Cu-64, Cu-67, Ag-105, Ag-106, Ag-112, Ag-113, Pt-185, Pt-186, Pt-187, Pt-188, Pt-189, Pt-190, Pt-19 and Pt-197. A, B, y, x and n are as defined above. In some embodiments, Prussian blue materials may include of Ag₄[Fe(CN)₆], Pb₂[Fe(CN)₆], Sn₂[Fe(CN)₆], Co[Cr(CN)₆]_{2/3} or any combination thereof.

As a non-limiting example of physical linkage, a metal isotope may be bounded by physical or physicochemical bonds, such as ion exchange, absorption, mechanical trapping. In some aspects, a metal isotope can be adsorbed on the surface of Prussian blue materials or incorporated into the vacancies of Prussian blue materials.

In certain embodiments, compositions disclosed herein may comprise a metal isotope emitting any kind of radiation known in the field. In some aspects, the radiation may be alpha, beta, gamma, positron radiation, or a combination thereof. In some embodiments, compositions disclosed herein may comprise a metal isotope emitting alpha or beta radiation. In some aspects, a metal isotope emitting alpha or beta radiation may be Sc-47, Sc-48, Cu-67, Zn-69, Rb-86, Rb-84, Y-90, Zr-95, Zr-97, Nb-95, Nb-96, Nb-98, Ag-112, Ag-113, Cd-115, Cd-117, Cd-118, Cs-136, Cs-138, La-140, La-141, La-142, Sm-153, Eu-150m, Eu-152m, Eu-158, Tb-149, Dy-165, Dy-166, Ho-164, Ho-166, Ho-167, Hf-183, Ta-183, Ta-184, Ta-185, Re-186, Re-188, Re-189, Os-191, Os-193, Os-194, Os-195, Os-196, Ir-193, Ir-195, Pt-197, Pt-200, Au-196, Au-199, Hg-203, Hg-208, Pb-209, Pb-212, Bi-212, Bi-213, or any combination thereof.

In some other embodiments, compositions disclosed herein may comprise a metal isotope emitting gamma or positron radiation. In some aspects, a metal isotope emitting gamma or positron radiation may be Sc-43, Sc-44, Cu-61, Cu-64, Zn-62, Zn-69m, Ga-67, Ga-68, Rb-81, Rb-82m, Y-84, Y-85, Y-86, Zr-86, Zr-87, Zr-88, Zr-89, Zr-90, Nb-88, Nb-89, Nb-90, Ag-105, Ag-106, Cd-104, Cd-105, Cd-107, Cd-111, Cs-127, Cs-129, Cs-131, Cs-134, Cs-135, La-131, La-132, La-133, La-135, Sm-141, Sm-142, Eu-145, Eu-146, Eu-147, Eu-152m, Tb-147, Tb-150, Tb-151, Tb-152, Tb-154, Tb-154m, Tb-156, Tb-156m, Dy-152, Dy-153, Dy-155, Dy-157, Ho-155, Ho-156, Ho-158, Ho-159, Ho-160, Ho-164, Hf-166, Hf-168, Hf-170, Hf-171, Hf-173, Hf-179, Ta-171, Ta-172, Ta-173, Ta-174, Ta-175, Ta-176, Ta-177, Ta-178, Re-181, Re-182, Re-183, Re-184, Re-186, Re-188, Re-190, Os-180, Os-181, Os-182, Os-183, Ir-183, Ir-184, Ir-185, Ir-186, Ir-187, Ir-188, Ir-189, Ir-190, Pt-185, Pt-186, Pt-187, Pt-188, Pt-189, Pt-190, Pt-191, Pt-197, Au-190, Au-191, Au-192, Au-193, Au-194, Au-196, Au-198, Au-199, Au-200, Au-201, Hg-190, Hg-191, Hg-193, Hg-197, Tl-194, Tl-195, Tl-196, Tl-197, Tl-198, Tl-199, Tl-200, Tl-201, Tl-202, Tl-203, Tl-204, Pb-206, Pb-207, Pb-208, Pb-209, Pb-210, Pb-211, Pb-212, Pb-213, Pb-214, Bi-200, Bi-201, Bi-201, Bi-203, Bi-204, Bi-205, Bi-206, or any combination thereof.

In certain embodiments, compositions disclosed herein may comprise soluble Prussian blue materials forming a particle. In some aspects, a particle formed of Prussian blue materials may be a nanoparticle. In other aspects, a particle formed of Prussian blue materials may be a microparticle. In still other aspects, a particle formed of Prussian blue materials may be about 1 nanometer (nm) to about 10 microns (μm). In some embodiments, a particle formed of Prussian blue materials for use herein can range from about 10 nm to about 300 nm (e.g., about 10 nm, about 25 nm, about 50 nM, about 75 nM, about 100 nm, about 125 nm, about 150 nm, about 175 nm, about 200 nm, about 225 nm, about 250 nM, about 275 nM, about 300 nm) in diameter.

In certain embodiments, compositions disclosed herein may comprise soluble Prussian blue materials synthesized by methods known in the art. In some aspects, a starting material can be a commercially available Prussian blue particle. In some aspects, a starting material may be commercially available from Radiogardase (by Heyltex). In other aspects, a starting material can be a Prussian blue particle synthesized from FeCl₃ and K₄[Fe(CN)₆] which may be acidified for example with organic or inorganic acids (such as HCl, citric acid, etc) which is mixed. In some aspects, the step of mixing the solution may be temperature and pH controlled. In some aspects, a temperature suitable for mixing the solution disclosed herein may be about 20° C., about 21° C., about 22° C., about 23° C., about 24° C., about 25° C., or about 26° C. In some aspects, a pH suitable for mixing the solution disclosed herein may be about 3, about 4, about 5, about 6, or about 7. In other aspects, one or more additives known in the field to aid in formation of Prussian blue particles with homogeneous size distribution and/or subsequent incorporation of a metal isotope and/or covering Prussian blue particles with a biocompatible coating as disclosed herein may be added during mixing of the solution disclosed herein.

In some embodiments, Prussian blue materials may be synthesized by reacting a metallic salt with a metal cyanide ([M′(CN)₆]³⁻) in a solvent. In some aspects, the metallic salt comprises, consists essentially of, or consists of a salt of salt of V, Cr, Mn, Fc, Co, Ni, Cu, Zn, Ga, Sr, Zr, Nb, Li, Na, K, Rb, Cs, Fr, TI, Mo, Ru, Rh, Pd, Ag, Cd, In, Lu, Ba, Hf, Ta, W, Os, Pt, Hg, La, Eu, Gd, Tb, Dy and Ho in any oxidation state thereof and in any combination thereof. In other aspects, the metallic salt comprises, consists essentially of, or consists of a metallic salt of a chloride, a nitrate, a nitrite, a sulfate, a fluorinate, a glutamate, an acetate, a carbonate, a citrate, a phosphate, a sulfate and any combination thereof. In still other aspects, the metal cyanide comprises, consists essentially of, or consists of a metal cyanide represented by [M′(CN)₆]³⁻, wherein M′ represents V, Cr, Mn, Fe, Co, Ni, Cu, Zn, Ga, Sr, Zr, Nb, Li, Na, K, Rb, Cs, Fr, TI, Mo, Ru, Rh, Pd, Ag, Cd, In, Lu, Ba, Hf, Ta, W, Os, Pt, Hg, La, Eu, Gd, Tb, Dy and Ho in any oxidation state thereof and in any combination thereof.

In some embodiments, the solvent in which the reaction between the metallic salt and the metallic cyanide described above occurs may not be particularly limited, so long as the reaction proceeds in this solvent. In some aspects, the solvent comprises, consists essentially of, or consists of water, air, or an organic solvent. In a preferred aspect, the solvent is ultrapure water. As used herein, the “ultrapure water” refers to “grade 1” water as defined by the International Organization for Standardization (ISO), with resistivity of 18.2 MΩ2·cm. As used herein, the terms “ultrapure water” and “Milli-Q water” are synonymous.

In some embodiments, the solvent in which the reaction between the metallic salt and the metallic cyanide described above occurs is an organic solvent. In some aspects, the organinc solvent can be hydrophilic to any degree or hydrophobic to any degree. In some aspects, the organic solvent comprises, consists essentially of, or consists of hexane; benzene; toluene; diethyl ether; chloroform; 1,4-dioxane; ethyl acetate; tetrahydrofuran (THF); dichloromethane; acetone; acetonitrile (MeCN); dimethylformamide (DMF); dimethyl sulfoxide (DMSO); a polar protic solvent; acetic acid; n-butanol; isopropanol; n-propanol; ethanol; methanol; formic acid; and any combination thereof, so long as the metallic salt and the metallic cyanide are sufficiently dissolved in the combination and the reaction proceeds in this combination of solvents.

### (b) Biocompatible Coatings

In certain embodiments, PBNP compositions disclosed herein may comprise a shell partially or completely encapsulating a nanoparticle. In some aspects, a shell may encapsulate about 25%, about 50%, about 75%, or about 100% of the nanoparticle. In some other aspects, a shell may completely encapsulate a nanoparticle formed of Prussian blue materials as disclosed herein.

In some embodiments, a shell may be comprised of a biocompatible coating. In some aspects, a biocompatible coating comprises one or more biocompatible materials assisting to in vivo and in vitro use of compositions disclose herein. In some embodiments, a biocompatible coating of the shell may comprise at least one material selected from the group consisting of dextran; chitosan; silica; polyethylene glycol (PEG); avidin; a proteins; a nucleic acids; a carbohydrates; a lipid; neutravidin; streptavidin; gelatin; collagen; fibronectin; albumin; a serum protein; lysozyme; a phospholipid; a polyvinyl pyrrolidone (PVP); a polyvinyl alcohol; a polyethylene glycol diacrylate; polyethylenimine (PEI); and a combination thereof. Without wishing to be bound to any particular theory, the biocompatible coating is believed to prevent the compositions from aggregating and to prevent leakage of ions from the core to the surrounding environment.

In some embodiments, a dextran of the biocompatible coating may comprise a dextran that is a complex, branched polysaccharide having chains of varying lengths, preferably chains having lengths of from about 3 to about 2000 kDa (e.g., about 3, 10, 50, 100, 200, 300, 400, 500, 600, 700, 800, 900, 1000, 1100, 1200, 1300, 1400, 1500, 1600, 1700, 1800, 1900, 2000). In other embodiments, a chitosan of the biocompatible coating may comprise a linear polysaccharide having randomly distributed units of β-(1-4)-linked D-glucosamine (deacetylated unit) and units of N-acetyl-D-glucosamine (acetylated unit). In still other embodiments, a silica of the biocompatible coating may comprise an oxide of silicon with the chemical formula SiO₂. In yet other embodiments, a polyethylene glycol (PEG) of the biocompatible coating may comprise polyethylene oxide (PEO) or polyoxyethylene oxide (POE). In other embodiments, an avidin of the biocompatible coating may comprise a protein produced in the oviducts of birds, reptiles and amphibians deposited in the whites of their eggs. In yet other embodiments, an albumin of the biocompatible coating may comprise bovine serum albumin (BSA, fraction V), human serum albumin (HSA) and all serum albumin derived from mammals. In an aspect, serum proteins of the biocompatible coating may comprise at least one member selected from the group consisting of Orosomucoid; antitrypsin; alpha-1 antichymotrypsin; alpha-2 macroglobulin (AMG); haptoglobin; transferrin; beta lipoprotein (LDL); immunoglobulin A (IgA); immunoglobulin M (IgM); immunoglobulin G (IgG); immunoglobulin E (IgE); and immunoglobulin D (IgD). In some embodiments, a lysozyme of the biocompatible coating may be of N-acetylmuramide glycanhydrolase. In still other embodiments, phospholipids of the biocompatible coating may comprise of all natural phospholipids and synthetic phospholipids. Non-limiting examples of natural phospholipids and synthetic phospholipids include DMPA, DPPA, DSPA DDPC, DLPC, DMPC, DPPC, DSPC, DOPC, POPC, DEPC DMPG, DPPG, DSPG, POPG DMPE, DPPE, DSPE DOPE DOPS mPEG-phospholipid, polyglycerin-phospholipid, functionalized-phospholipid, and terminal activated-phospholipid. In other embodiments, a polyvinyl pyrrolidone (PVP) of the biocompatible coating may comprise a polymer made from repeating monomer N-vinylpyrrolidone units. In some aspects, the molecular weight of the PVP is not particularly limited, as long as the PVP is suitable for use in the biocompatible coating of the present disclosure. As used herein, the terms “polyvidone” and “povidone” are synonymous with PVP. In some embodiments, a polyvinyl alcohol of the biocompatible coating may comprise PVOH, PVA, and PVAI. In some aspects, molecular weights of the PVOH, PVA, and PVAI are not particularly limited, as long as the PVOH, PVA, and PVAI are suitable for use in the biocompatible coating of the present disclosure. In other embodiments, a polyethylene glycol diacrylate of the biocompatible coating may comprise a polyethylene glycol terminated with acrylate groups. In some aspects, molecular weight of the polyethylene glycol diacrylate is not particularly limited, as long as the polyethylene glycol diacrylate is suitable for use in the biocompatible coating of the present disclosure. In some embodiments, lipids of the biocompatible coating may comprise sterols, fats, oils, waxes, vitamin A, vitamin D, vitamin E, vitamin K, phospholipids, (mono-, di-, tri-) glycerides, or any combination thereof.

In certain embodiments, a biocompatible coating of the shell of PBNP compositions disclosed herein may comprise one or more polymers. In some aspects, a polymer suitable for use in a biocompatible coating disclosed herein may be polyethylene glycol, polypropylene glycol, polyoxyethylene ether, polyanethol sulfonic acid, polyethylene imine, polymalcimide, polyvinyl alcohol, polyvinyl chloride, polyvinyl acetate, polyvinyl pyrrolidone, polyvinyl sulfate, polyacrylic acid, polymethacrylic acid, polylactide, polylactide glycide, or a combination thereof. In a preferred aspect, the biocompatible coating comprises polyethylene imine (PEI).

In certain embodiments, the biocompatible coating can be applied to the core of the compositions disclosed herein by a variety of physical and chemical interactions including, but not limited to: electrostatic (charge-based), covalent, hydrophobic and van der Waal's interactions. In some embodiments, the biocompatible coating is applied by suspending the core in a solution comprised of one or more materials selected from the group consisting of dextran; chitosan; silica; polyethylene glycol (PEG); avidin; a proteins; a nucleic acids; a carbohydrates; a lipid; neutravidin; streptavidin; gelatin; collagen; fibronectin; albumin; a serum protein; lysozyme; a phospholipid; a polyvinyl pyrrolidone (PVP); a polyvinyl alcohol; a polyethylene glycol diacrylate; polyethylenimine (PEI); and a combination thereof.

### (c) Biomolecules

In certain embodiments, PBNP compositions disclosed herein may comprise a shell partially or completely encapsulating a nanoparticle with biocompatible coating wherein at least one biomolecule may be attached to, or absorbed to, the biocompatible coating. In some aspects, the shell may completely encapsulate a nanoparticle formed of Prussian blue materials with biocompatible coating wherein at least one biomolecule may be attached to, or absorbed to, the biocompatible coating.

In some embodiments, a biocompatible coating disclosed herein may absorb at least about 25%, at least about 50%, or at least about 75% biomolecule weight by total weight of the biocompatible coating. In some other embodiments, at least about 25%, at least about 50%, at least about 75%, at least about 100% of the outer surface of the biocompatible coating has biomolecules attached. In some aspects, about 25% to about 100% (e.g., about 25%, 30%, 35%, 40%, 45%, 50%, 55%, 60%, 65%, 70%, 75%. 80%, 85%, 90%, 95%, 99%, 100%) of the outer surface of the biocompatible coating has biomolecules attached.

In certain embodiments, a biomolecule attached to, or absorbed to, the biocompatible coating may comprise an antibody, a peptide, a protein, an enzyme, an amino acid, a nucleic acid, a carbohydrate, a fat, an aptamer, a small molecule, a synthetic molecule or a combination thereof.

In some embodiments, a nucleic acid may be DNA (deoxyribonucleic acid), RNA (ribonucleic acid), a peptide nucleic acid, a morpholino-nucleic acid, a locked nucleic acid, a glycol nucleic acid, a threose nucleic acid, an oligonucleotide, or a combination thereof.

In some other embodiments, at least one of the biomolecules may be a peptide. In some embodiments, a peptide may consist of any sequence of 50 amino acids or less, excluding zero. In another aspect, a peptide may consist of any sequence of about 2 amino acids to about 50 amino acids. In a preferred aspect, a peptide may consist of any sequence of 20 amino acids or less, excluding zero.

In certain embodiments, at least one of the biomolecules may be an antibody. As used herein, an “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, nanobodies, single variable domain (e.g., variable heavy domain, variable light domain), bispecific, camelids, and peptabodies.

### (i) CD137 and CD137 Agonists

CD137 (also known as 4-1BB or TNFRSF9) is a glycoprotein that is a member of the tumor necrosis factor receptor superfamily. CD137 binds to a high-affinity ligand (CD137L, also known as 4-1BBL or TNFSF9) expressed on antigen-presenting cells such as, but not limited to, dendritic cells, macrophages, and activated B cells. Expression of CD137 is found on various hematopoietic cells, including primed T cells, natural killer (NK) cells, neutrophils, monocytes, dendritic cells, mast cells, and the like. CD137 can be a costimulatory molecule for T-cell activation and engagement of CD137 on T cells by natural ligand or agonist monoclonal antibody (mAb) can enhance T-cell proliferation and provide protection to CD8 T cells from activation-induced cell death through nuclear factor κB-mediated activation and up-regulation of the antiapoptotic Bcl-2 family members.

In certain embodiments, a biomolecule disclosed herein may be an agonist for CD137. As used herein, the term “agonist” refers to any molecule that partially or fully promotes, induces, increases, and/or activates a biological activity of a native polypeptide disclosed herein (e.g., CD137). Suitable agonist molecules can include, but are not limited to, agonist antibodies or antibody fragments, fragments or amino acid sequence variants of native polypeptides, peptides, antisense oligonucleotides, small organic molecules, small molecules, and the like. In some embodiments, activation in the presence of the agonist is observed in a dose-dependent manner. In some embodiments, the measured signal (e.g., biological activity) may be at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% higher than the signal measured with a negative control under comparable conditions. Also disclosed herein, are methods of identifying agonists suitable for use in the methods of the disclosure. For example, these methods include, but are not limited to, binding assays such as enzyme-linked immuno-absorbent assay (ELISA), Forte Bio systems, and radioimmunoassay (RIA). These assays determine the ability of an agonist to bind the polypeptide of interest (e.g., a receptor or ligand, e.g., CD137) and therefore indicate the ability of the agonist to promote, increase or activate the activity of the polypeptide. Efficacy of an agonist herein can also be determined using functional assays, such as the ability of an agonist to activate or promote the function of the polypeptide. For example, a functional assay may comprise contacting a polypeptide with a candidate agonist molecule and measuring a detectable change in one or more biological activities normally associated with the polypeptide. The potency of an agonist is usually defined by its EC₅₀ value (i.e., the concentration required to activate 50% of the agonist response). The lower the EC₅₀ value the greater the potency of the agonist and the lower the concentration that is required to activate the maximum biological response.

In certain embodiments, a biomolecule disclosed herein may be an aptamer capable of binding CD137. Aptamers are single-stranded oligonucleotides designed through an enrichment process to develop a unique structure capable of binding a given target protein, usually for therapeutic purposes. In some embodiments, a biomolecule disclosed herein may be an aptamer or multiple aptamers capable of activating a biological activity of native CD137.

In certain embodiments, a biomolecule disclosed herein may be a ligand of CD137 (i.e., CD137L). In some embodiments, a biomolecule disclosed herein may be recombinant CD137L or a fragment thereof. In some embodiments, a biomolecule disclosed herein may be recombinant CD137L or a fragment thereof capable of activating a biological activity of native CD137.

In certain embodiments, a biomolecule disclosed herein may be an agonist CD137 antibody and/or agonist CD137 antibody fragments. Accordingly, in some embodiments, an anti-CD137 agonist antibody, or antigen-binding fragment thereof, as described herein, may bind to and agonize CD137 and/or allow or promotes CD137L binding. In some embodiments, an anti-CD137 agonist antibody, or antigen-binding fragment thereof, described herein, binds to and agonizes CD137. In some embodiments, anti-CD137 antibodies provided by the present disclosure may bind to and agonize CD137 and modulate one or more activities of a cell expressing CD137, wherein said modulation is an increase or decrease in the activity of said cell. In some aspects, CD137 antibody agonists disclosed herein may modulate one or more activities of a T cell expressing CD137. The term “T cell” herein can refers to a type of white blood cell that can be distinguished from other white blood cells by the presence of a T cell receptor on the cell surface. There are several subsets of T cells, including, but not limited to, T helper cells (such as T_{H} cells or CD4⁺ T cells) and subtypes, including T_{H}1, T_{H}2, T_{H}3, T_{H}17, T_{H}9, and T_{FH} cells, cytotoxic T cells (i.e., T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (such as T_{reg} cells or suppressor T cells) and subtypes, including CD4⁺ FOXP3⁺ T_{reg} cells, CD4⁺ FOXP3⁻ T_{reg} cells, Tr1 cells, Th3 cells, and T_{reg}17 cells, natural killer T cells (such as. NKT cells), mucosal associated invariant T cells (MAITs), and gamma delta T cells (γδ T cells), including Vγ9/Vδ2 T cells. Any one or more of the aforementioned or unmentioned T cells may be the target cell type for any of the compositions disclosed herein.

In some embodiments, an anti-CD137 agonist antibody, or antigen-binding fragment thereof, as disclosed herein may activate T cells. As used herein, the term “T cell activation” or “activation of T cells” refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. T cell activation requires at least two signals to become fully activated. The first occurs after engagement of the T cell antigen-specific receptor (TCR) by the antigen-major histocompatibility complex (MHC), and the second by subsequent engagement of co-stimulatory molecules (e.g., CD28). These signals are transmitted to the nucleus and result in clonal expansion of T cells, upregulation of activation markers on the cell surface, differentiation into effector cells, induction of cytotoxicity or cytokine secretion, induction of apoptosis, or a combination thereof. In some embodiments, anti-CD137 antibody agonists disclosed herein may result in at least one T cell-mediated response. As used herein, the term “T cell-mediated response” refers to any response mediated by T cells, including, but not limited to, effector T cells (e.g., CD8⁺ cells) and helper T cells (e.g., CD4⁺ cells). T cell mediated responses include, for example, T cell cytotoxicity and proliferation.

In some embodiments, an anti-CD137 agonist antibody, or antigen-binding fragment thereof as disclosed herein may increase the activity of a CD4+ or CD8+ effector cell, decrease the activity of, or deplete, a regulatory T cell (T reg), or both. In either case, the net effect of the antibody can be an increase in the activity of effector T cells, particularly CD4+, CD8+or NK effector T cells. Methods for determining a change in the activity of effector T cells are well known and are described herein. In some embodiments, anti-CD137 antibody agonists disclosed herein may cause an increase in activity in a CD8+ T cell, optionally wherein said increase in activity is an increase in proliferation, IFN-γ production, and/or IL-2 production by the T cell. In some aspects, the increase can be at least about 2-fold, at least about 10-fold, or at least about 25-fold higher than the change in activity caused by an isotype control antibody measured in the same assay.

In certain embodiments, an anti-CD137 agonist antibody, or antigen-binding fragment thereof as disclosed herein may have affinity for CD137 in its native state. In some aspects, anti-CD137 agonist antibodies disclosed herein may bind to a CD137 localized on the surface of a cell. By “localised on the surface of a cell” it is meant that CD137 is associated with the cell such that one or more region of CD137 is present on the outer face of the cell surface. For example, CD137 may be inserted into the cell plasma membrane (i.e., orientated as a transmembrane protein) with one or more regions presented on the extracellular surface. This may occur in the course of expression of CD137 by the cell. Thus, in one embodiment, “localised on the surface of a cell” may mean “expressed on the surface of a cell.” Alternatively, CD137 may be outside the cell with covalent and/or ionic interactions localising it to a specific region or regions of the cell surface. In some embodiments, anti-CD137 agonist antibodies disclosed herein may bind to a CD137 localized on the surface of a cancer cell.

In certain embodiments, a biomolecule disclosed herein may have specificity for CD137. As used herein “specificity for CD137” refers to a biomolecule capable of binding to CD137 in vivo (e.g., under physiological conditions). In some embodiments, an anti-CD137 agonist antibody, or antigen-binding fragment thereof as disclosed herein may have specificity for human CD137. In some aspects, anti-CD137 agonist antibodies of the present disclosure which binds human CD137 may also have cross-reactivity with another species of CD137, another member of the tumor necrosis factor receptor superfamily, or both. As used herein, the term antigen “cross-presentation” refers to presentation of exogenous protein antigens to T cells via MHC class I and class II molecules on APCs. Cross-reactivity can be measured by detecting a specific reactivity with purified antigen in binding assays (e.g., SPR, ELISA) or binding to, or otherwise functionally interacting with, cells physiologically expressing CD137. Methods for determining cross-reactivity include standard binding assays as described herein, for example, by Biacore surface plasmon resonance (SPR) analysis using a Biacore 2000 SPR instrument, or flow cytometric techniques.

In certain embodiments, a biomolecule disclosed herein may be an anti-CD137 agonistic monoclonal antibody. Non-limiting examples of anti-CD137 agonistic monoclonal antibodies suitable for use herein can include, urelumab (BMS-663513); AGEN2373; ADG106; EU101; utomilumab (PF-05082566); and the like. One of skill in the art can appreciate that anti-CD137 agonistic monoclonal antibodies can be designed and synthesized by a variety of methods. Accordingly, in some embodiments a biomolecule disclosed herein may be an anti-CD137 agonistic monoclonal antibody designed, synthetized, and/or optimized for inclusion with the biofunctionalized nanocomposites disclosed herein.

### (d) Biofunctionalized Nanocomposites

In certain embodiments, the present disclosure provides compositions comprising a biofunctionalized nanocomposite. As used herein, the term “nanocomposite” refers to a composition comprised of a nanoparticle core partially or completely surrounded with a material. As used herein, the term “biofunctionalized nanocomposite” refers to a nanocomposite that has been modified to add at least one biological function.

In certain embodiments, biofunctionalized nanocomposites of the present disclosure comprise a core comprising a nanoparticle formed of Prussian blue materials and a shell obtained by partially or completely encapsulating the Prussian blue core with a biocompatible coating wherein at least one biomolecule is attached to, or absorbed to, the biocompatible coating.

In certain embodiments, biofunctionalized nanocomposites disclosed herein may comprise soluble Prussian blue materials forming a nanoparticle prepared as described herein. In some embodiments, the nanoparticle may be formed of one or more of the Prussian blue materials disclosed herein. In some aspects, the Prussian blue materials may be an iron hexacyanoferrate (II) compound as disclosed herein. In some aspects, the Prussian blue materials may be represented by any one of general formulas (I), (II), or (III) as provided herein.

In some embodiments, a nanoparticle formed of Prussian blue materials suitable for use in the biofunctionalized nanocomposites disclosed herein may be about 1 nanometer (nm) to about 10 microns (μm). In some aspects, a particle formed of Prussian blue materials may be about 5 nm to about 500 μm (e.g., about 5 nm, about 10 nm, about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 5 μm, about 10 μm, about 50 μm, about 100 μm, about 150 μm, about 200 μm, about 250 μm, about 300 μm, about 350 μm, about 400 μm, about 450 μm, about 500 μm). In some other aspects, a particle formed of Prussian blue materials may be about 10 nm to about 1 μm.

In certain embodiments, biofunctionalized nanocomposites disclosed herein may comprise a core comprising a nanoparticle formed of Prussian blue materials and a shell obtained by partially or completely encapsulating the Prussian blue core with a biocompatible coating. In some aspects, a shell may encapsulate about 25%, about 50%, about 75%, or about 100% of the nanoparticle. In a preferred aspect, the shell completely encapsulates a nanoparticle formed of Prussian blue materials as disclosed herein.

In certain embodiments, biofunctionalized nanocomposites disclosed herein may comprise a biocompatible coating prepared as described herein. In some aspects, a shell may be formed of one or more of the biocompatible coating materials disclosed herein. In other aspects, a biocompatible coating suitable for use in a biofunctionalized nanocomposite disclosed herein may be dextran, chitosan, silica, polyethylene glycol (PEG), avidin, a protein, a nucleic acid, a carbohydrate, a lipid, neutravidin, streptavidin, gelatin, collagen, fibronectin, albumin, a serum protein, a lysozyme, a phospholipid, a polyvinyl pyrrolidone (PVP), a polyvinyl alcohol, polyethylene glycol diacrylate, polyethylenimine (PEI), or a combination thereof. In other aspects, a biocompatible coating may comprise PEI having an average molecular weight (MW) in the range from about 100 daltons to about 100,000 daltons. In some aspects, PEI may have an average molecular weight of about 100 daltons, about 500 daltons, about 1,000 daltons, about 1,500 daltons, about 2,000 daltons, about 2,500 daltons, about 3,000 daltons, about 3,500 daltons, about 4,000 daltons, about 4,500 daltons, about 5,000 daltons, about 6,000 daltons, about 7,000 daltons, about 8,000 daltons, about 9,000 daltons, about 10,000 daltons, about 20,000 daltons, about 30,000 daltons, about 40,000 daltons, about 50,000 daltons, about 60,000 daltons, about 70,000 daltons, about 80,000 daltons, about 90 000 daltons, or about 100,000 daltons. In yet other aspects, a biocompatible coating may comprise PEI polymers of at least two different average molecular weights ranging from about 100 daltons to about 100,000 daltons. In other aspects, a biocompatible coating may comprise PEI polymers of at least two different average molecular weights wherein there molecular weight may be about 100 daltons, about 500 daltons, about 1,000 daltons, about 1,500 daltons, about 2,000 daltons, about 2,500 daltons, about 3,000 daltons, about 3,500 daltons, about 4,000 daltons, about 4,500 daltons, about 5,000 daltons, about 6,000 daltons, about 7,000 daltons, about 8,000 daltons, about 9,000 daltons, about 10,000 daltons, about 20,000 daltons, about 30,000 daltons, about 40,000 daltons, about 50,000 daltons, about 60,000 daltons, about 70,000 daltons, about 80,000 daltons, about 90 000 daltons, about 100,000 daltons, or a combination thereof.

In certain embodiments, biofunctionalized nanocomposites disclosed herein may comprise a shell partially or completely encapsulating a nanoparticle with biocompatible coating wherein at least one biomolecule may be attached to, or absorbed to, the biocompatible coating. In some embodiments, the biocompatible coating disclosed herein may absorb at least about 25%, at least about 50%, or at least about 75% biomolecule weight by total weight of the biocompatible coating. In other embodiments, at least about 25%, at least about 50%, at least about 75%, or at least about 100% of the outer surface of the biocompatible coating comprises one or more attached biomolecules.

In certain embodiments, biomolecules may be one or more of the biomolecules disclosed herein. In certain embodiments, biofunctionalized nanocomposites disclosed herein may comprise a shell partially or completely encapsulating a nanoparticle with biocompatible coating wherein at least one CD137 agonist may be attached to, or absorbed to, the biocompatible coating. In some aspects, a CD137 agonist suitable for use in a biofunctionalized nanocomposite disclosed herein may be an antibody, a peptide, a protein, an enzyme, an amino acid, a nucleic acid, a carbohydrate, a fat, an aptamer, a small molecule, a synthetic molecule or a combination thereof. In some aspects, a CD137 agonist suitable for use in a biofunctionalized nanocomposite disclosed herein may be an anti-CD137 antibody agonist or antibody fragment thereof.

In certain embodiments, biomolecules disclosed herein (e.g., an CD137 agonist, anti-CD137 antibody agonist) may be attached to, or absorbed to, the biocompatible coating biofunctionalized nanocomposites herein either directly or indirectly, by any suitable means. In some aspects, the biomolecule (e.g., an CD137 agonist, anti-CD137 antibody agonist) can be directly covalently coupled (e.g., such as by a metal-thiol bond) to a nanocomposite herein. In some other aspects, the biomolecule (e.g., an CD137 agonist, anti-CD137 antibody agonist) can be coupled to a nanocomposite herein by using a “linker” molecule, so long as the linker does not significantly negatively affect the activity of the enzyme or the function of the biomolecule. The linker preferably is biocompatible. Common molecular linkers known in the art include, but are not limited to, a maleimide or succinimide group, streptavidin, neutravidin, biotin, or similar compounds.

In some embodiments, one or more biomolecules disclosed herein may be conjugated to a nanoparticle formed of one or more of the Prussian blue materials disclosed herein. The terms “linked”, “joined”, “grafted”, “tethered”, “associated”, “bioconjugated”, and “conjugated” in the context of the nanoparticles of the invention, are used interchangeably to refer to any method known in the art for functionally connecting moieties (such as biologically active agents, surfactants, or targeting moieties), including, without limitation, recombinant fusion, covalent bonding, disulfide bonding, ionic bonding, hydrogen bonding, and electrostatic bonding. In some aspects, biomolecules to be conjugated to nanoparticles disclosed herein may be a CD137 agonist. In some aspects, a CD137 agonist suitable for bioconjugation may be a peptide, an antibody, an aptamer, a polynucleotide, an oligo, a messenger RNA (mRNA) sequence, or any combination thereof. In some embodiments, one or more anti-CD137 antibody agonists disclosed herein may be bioconjugated to a nanoparticle formed of one or more of the Prussian blue materials disclosed herein. In some aspects, anti-CD137 antibodies conjugated to a nanoparticle disclosed herein may be, but are not limited to, an antibody, an antibody fragment, an affibody, a peptide, a cyclic peptide, a toxin, a small molecule, a recombinant humanized monoclonal antibody, a rabbit antibody, a goat antibody, a mouse antibody, an anti-hapten antibody, or any combination thereof. In some other aspects, anti-CD137 antibodies conjugated to a nanoparticle disclosed herein may include a non-protein substance (e.g., via covalent conjugation). For example, an anti-CD137 antibody may, but is not limited to include a radio-isotope, a cytotoxic molecule, a covalently-attached polymer, and the like.

In some embodiments, one or more anti-CD137 antibody agonists disclosed herein may be bioconjugated to a nanoparticle by covalent synthesis. In some aspects, one or more anti-CD137 antibody agonists disclosed herein may be crosslinked to nanoparticles disclosed herein. Cross-linking can be carried out using a variety of crosslinking agents to crosslink the biomolecules (e.g., anti-CD137 antibody agonists) disclosed herein to nanoparticles disclosed herein. Any suitable crosslinker can be used, including homobifunctional crosslinkers (having identical reactive groups at either end of a spacer arm) and heterobifunctional crosslinkers (having different reactive groups at either end). Typical reactive groups at each end of a crosslinker include N-hydroxysuccinimidyl esters, imidoesters, maleimides, pyridyldithiols, haloacetyls, azides, diazirines, carbodiimides, and isocyanates. Exemplary crosslinkers include the carbodiimides, such as 1-ethyl-3-(3-dimethylaminopropyl) carbodiimide (EDC or EDAC), N,N′-dicyclohexylcarbodlimide (DCC), and N,N′-diisopropylcarbodiimide (DIC). Carbodiimide couplings may be conducted in the further presence of N-hydroxysuccinimide or N-hydroxysulfosuccinimide, which may improve the efficiency of carbodiimide coupling reactions. Other crosslinkers include amine-to-amine crosslinkers such as disuccinimidyl glutarate (DSG), disuccinimidyl suberate (DSS), bis(2-(succinimidooxycarbonyloxy)ethyl)sulfone (BSOCOES), dimethyl suberimidate (DMS), 1,5-difluoro-2,4-dinitrobenzene (DFDNB), dithiobis(succinimidyl propionate) (DSP), tris-(succinimidyl)aminotriacetate (TSAT), dimethyl pimelimidate (DMP), bis(sulfosuccinimidyl)suberate (BS3), ethylene glycol bis (succinimidyl succinate) (EGS), ethylene glycol bis (sulfosuccinimidyl succinate) (sulfo-EGS), disuccinimidyl tartrate (DST), dimethyl 3,3′-dithiobispropionimidate (DTBP), PEGylated bis(sulfosuccinimidyl)suberate (e.g., BS (PEG) 5 and BS(PEG)9), 3,3′-dithiobis(sulfosuccinimidyl propionate) (DTSSP), and dimethyl adipimidate (DMA). Other crosslinkers include amine-to-sulfhydryl crosslinkers such as sulfo-SIAB (sulfosuccinimidyl (4-iodoacetyl)aminobenzoate), SM(PEG)6 (PEGylated, long-chain SMCC crosslinker), SMPT (4-succinimidyloxycarbonyl-alpha-methyl-α(2-pyridyldithio)toluene), SIAB (succinimidyl (4-iodoacetyl)aminobenzoate), sulfo-SMCC (sulfosuccinimidyl 4-(N-malcimidomethyl)cyclohexane-1-carboxylate), BMPS (N-β-malcimidopropyl-oxysuccinimide ester), SM(PEG)12 (PEGylated, long-chain SMCC crosslinker), MBS (m-malcimidobenzoyl-N-hydroxysuccinimide ester), SMPB (succinimidyl 4-(p-maleimidophenyl) butyrate), succinimidyl 3-(2-pyridyldithio)propionate (SPDP), sulfo-LC-SPDP (sulfosuccinimidyl 6-(3′-(2-pyridyldithio)propionamido)hexanoate), sulfo-EMCS (N-E-malcimidocaproyl-oxysulfosuccinimide ester), LC-SMCC (succinimidyl 4-(N-malcimidomethyl) cyclohexane-1-carboxy-(6-amidocaproate)), GMBS (N-γ-maleimidobutyryl-oxysuccinimide ester), SM(PEG)8 (PEGylated, long-chain SMCC crosslinker), sulfo-GMBS (N-γ-malcimidobutyryl-oxysulfosuccinimide ester), sulfo-SMPB (sulfosuccinimidyl 4-(N-maleimidophenyl)butyrate), sulfo-KMUS (N-K-malcimidoundecanoyl-oxysulfosuccinimide ester), SMPH (succinimidyl 6-((beta-malcimidopropionamido)hexanoate)), SM(PEG)4 (PEGylated SMCC crosslinker), AMAS (N-α-maleimidoacet-oxysuccinimide ester), SMCC (succinimidyl 4-(N-malcimidomethyl)cyclohexane-1-carboxylate), sulfo-MBS (m-malcimidobenzoyl-N-hydroxysulfosuccinimide ester), PEG12-SPDP (PEGylated, long-chain SPDP crosslinker), SMCC (succinimidyl trans-4-(maleimidylmethyl)cyclohexane-1-carboxylate), LC-SPDP (succinimidyl 6-(3(2-pyridyldithio)propionamido)hexanoate), EMCS (N-ε-malemidocaproyl-oxysuccinimide ester), SBAP (succinimidyl 3-(bromoacetamido)propionate), SPDP (succinimidyl 3-(2-pyridyldithio)propionate), PEG4-SPDP (PEGylated, long-chain SPDP crosslinker), SIA (succinimidyl iodoacetate), SM(PEG)2 (PEGylated SMCC crosslinker), and SM(PEG)24 (PEGylated, long-chain SMCC crosslinker). Other crosslinkers include sulfhydryl-to-sulfhydryl crosslinkers such as tris (2-malcimidocthyl) amine (TMEA), bismalcimidohexane (BMH), 1,11-bismalcimido-tricthyleneglycol (BM (PEG) 3), 1,4-bismalcimidobutane (BMB), 1,8-bismaleimido-diethyleneglycol (BM (PEG) 2), bismalcimidocthane (BMOE), and dithiobismalcimidocthane (DTME). Further crosslinkers include sulfhydryl-to-carbohydrate crosslinkers such as 4-(4-N-malcimidophenyl) butyric acid hydrazide (MPBH), N-β-malcimidopropionic acid hydrazide (BMPH), N-K-malcimidoundecanoic acid hydrazide (KMUH), 3-(2-pyridyldithio) propionyl hydrazide (PDPH), and N-E-maleimidocaproic acid hydrazide (EMCH).

In some embodiments, about 50% to about 100% of anti-CD137 antibodies disclosed herein may be conjugated to a nanoparticle during synthesis. In some aspects, biofunctionalized nanocomposites disclosed herein may comprise the nanocomposites disclosed herein and anti-CD137 antibodies at a ratio that completely binds all anti-CD137 antibodies to the nanocomposites. In some other aspects, biofunctionalized nanocomposites disclosed herein may comprise the nanocomposites disclosed herein and anti-CD137 antibodies at a ratio that completely binds at least about 50% to about 99% (e.g., about 50%, 60%, 70%, 80%, 90%, 95%, 99%) of the anti-CD137 antibodies to the nanocomposites. In some embodiments, biofunctionalized nanocomposites disclosed herein may comprise the nanocomposites disclosed herein and anti-CD137 antibodies at a ratio of about 1 nanoparticle to about 1 anti-CD137 antibody (1:1), about 1.5 nanoparticles to about 1 anti-CD137 antibody (1.5:1), about 2 nanoparticles to about 1 anti-CD137 antibody (2:1), about 2.5 nanoparticles to about 1 anti-CD137 antibody (2.5:1), about 3 nanoparticles to about 1 anti-CD137 antibody (3:1), about 4 nanoparticles to about 1 anti-CD137 antibody (4:1), or about 5 nanoparticles to about 1 anti-CD137 antibody (1:1).

In certain embodiments, biofunctionalized nanocomposites disclosed herein may further comprise an imaging agent. In some aspects, an imaging agent may be one or more of the imaging agents disclosed herein. In some aspects, an imaging agent for use in a biofunctionalized nanocomposite disclosed herein may be a fluorescein compound, a rhodamine compound, a xanthene compound, a cyanine compound, a naphthalene compound, a coumarin compound, an oxadiazole compound, a pyrene compound, an oxazine compound, an acridine compound, an arylmethine compound, a tetrapyrrole compound, a proprietary molecule, or a combination thereof.

In certain embodiments, a biofunctionalized nanocomposite disclosed herein can be stable. As used herein, a biofunctionalized nanocomposite is considered to be “stable” when the neither the heating ability nor the hydrodynamic diameter of the composition have changed from baseline measurements. In some embodiments, a biofunctionalized nanocomposite disclosed herein can be stable from about 20° C. to about 120° C. In some aspects, a biofunctionalized nanocomposite can be stable at no less than about 20° C., about 40° C., about 60° C., about 80° C., about 100° C., or about 120° C. In some other aspects, a biofunctionalized nanocomposite can be stable no less than about 80°° C.

In certain embodiments, a biofunctionalized nanocomposite disclosed herein can be stable from about 1 day to about 14 days. In some aspects, a biofunctionalized nanocomposite can be stable for about 1 day, about 2 days, about 3 days, about 5 days, about 7 days, about 10 days, about 12 days, or about 14 days. In some other aspects, a biofunctionalized nanocomposite can be stable for about 7 days. In yet some other aspects, a biofunctionalized nanocomposite disclosed herein can be stable from about 0° C. to about 90° C. In some aspects, a biofunctionalized nanocomposite can be stable at about 0° C., about 10° C., about 20°° C., about 30° C., about 40° C., about 50° C., about 60° C., about 70° C., about 80° C., or about 90° C. In some other aspects, a biofunctionalized nanocomposite can be stable at about 4° C., about 20° C., or about 80° C. In some embodiments, a biofunctionalized nanocomposite disclosed herein can be stable from about 0° C. to about 90° C. for up to about 14 days. In some aspects, a biofunctionalized nanocomposite disclosed herein can be stable from about 0° C. to about 90° C. for about 3 days, about 5 days, about 7 days, about 10 days, about 12 days, or about 14 days. In some other aspects, a biofunctionalized nanocomposite disclosed herein can be stable at about 0° C., about 10° C., about 20° C., about 30° C., about 40° C., about 50° C., about 60° C., about 70° C., about 80° C., or about 90° C. for about 3 days, about 5 days, about 7 days, about 10 days, about 12 days, or about 14 days. In preferred aspects, a biofunctionalized nanocomposite can be stable at about 4° C., about 20° C., or about 80° C. for about 7 days.

### (e) Photothermal Therapy Agents

In certain embodiments, compositions disclosed herein comprise a photothermal therapy agent. As used herein, the term “photothermal therapy agent” refers to a composition comprised of light-absorbing materials suitable for use in photothermal therapy.

In some embodiments, photothermal therapy agents of the present disclosure may comprise a core comprising a nanoparticle formed of Prussian blue materials and a shell obtained by partially or completely encapsulating the Prussian blue core with a biocompatible coating wherein at least one biomolecule (e.g., a CD137 agonist) attached to, or absorbed to, the biocompatible coating.

In some embodiments, photothermal therapy agents disclosed herein may comprise soluble Prussian blue materials forming a nanoparticle prepared as described herein. In some aspects, the nanoparticle may be formed of one or more of the Prussian blue materials disclosed herein. In some aspects, the Prussian blue materials may be iron hexacyanoferrate (II) compounds as disclosed herein. In some other aspects, the Prussian blue materials may be represented by general formula (I) or (II) as disclosed herein.

In some embodiments, a nanoparticle formed of Prussian blue materials that is suitable for use in photothermal therapy agents disclosed herein may be about 1 nanometer (nm) to about 10 microns (μm). In still other aspects, a particle formed of Prussian blue may be about 5 nm to about 500 nm. In preferred aspects, a particle formed of Prussian blue materials may be about 10 nm to about 1 μm.

In some embodiments, photothermal therapy agents disclosed herein may comprise a core comprising a nanoparticle formed of Prussian blue materials and a shell obtained by partially or completely encapsulating the Prussian blue core with a biocompatible coating. In some aspects, a shell encapsulates about 25%, about 50%, about 75%, or about 100% of the nanoparticle. In a preferred aspect, a shell completely encapsulates a nanoparticle formed of Prussian blue materials as disclosed herein.

In certain embodiments, photothermal therapy agents disclosed herein may comprise a biocompatible coating prepared as described herein. In some aspects, a shell may be formed of one or more of the biocompatible coating materials disclosed herein. In a preferred aspect, a biocompatible coating suitable for photothermal therapy agents as disclosed herein may be dextran, chitosan, silica, polyethylene glycol (PEG), avidin; a protein, a nucleic acid, a carbohydrate, a lipid, neutravidin, streptavidin, gelatin, collagen, fibronectin, albumin, a serum protein, a lysozyme, a phospholipid, a polyvinyl pyrrolidone (PVP), a polyvinyl alcohol, polyethylene glycol diacrylate, polyethylenimine (PEI), or a combination thereof.

In some embodiments, photothermal therapy agents disclosed herein may comprise a shell partially or completely encapsulating a nanoparticle with biocompatible coating wherein at least one biomolecule may be attached to, or absorbed to, the biocompatible coating. In various embodiments, the biocompatible coating disclosed herein may absorb at least 25%, at least 50%, or at least 75% amount of biomolecule by total weight of the biocompatible coating. In other embodiments, at least 25%, at least 50%, at least 75%, at least 100% of the outer surface of the biocompatible coating comprises attached biomolecules.

In some embodiments, one or more biomolecules disclosed herein suitable for photothermal therapy agents disclosed herein may be an antibody, a peptide, a protein, an enzyme, an amino acid, a nucleic acid, a carbohydrate, a fat, an aptamer, a small molecule, a synthetic molecule or a combination thereof. In some aspects, a biomolecule suitable for photothermal therapy agents disclosed herein may be a CD137 agonist. In some other aspects, a biomolecule suitable for photothermal therapy agents disclosed herein is an anti-CD137 agonist antibody or antibody fragment thereof.

In other embodiments, photothermal therapy agents disclosed herein may further comprise an imaging agent. In some aspects, an imaging agent may be one or more of the imaging agents disclosed herein. In a preferred aspect, an imaging agent for use in photothermal therapy agents disclosed herein may be a fluorescein compound, a rhodamine compound, a xanthene compound, a cyanine compound, a naphthalene compound, a coumarin compound, an oxadiazole compound, a pyrene compound, an oxazine compound, an acridine compound, an arylmethine compound, a tetrapyrrole compound, a proprietary molecule, or a combination thereof.

In various embodiments, a photothermal therapy agent as disclosed herein can be stable. As used herein, a photothermal therapy agent is considered to be “stable” when the neither the heating ability nor the hydrodynamic diameter of particles comprising the composition have changed from baseline measurements.

In some embodiments, a photothermal therapy agent disclosed herein can be stable from about 20° C. to about 120° C. In an aspect, a photothermal therapy agent can be stable at no less than about 20° C., about 40° C., about 60° C., about 80° C., about 100° C., or about 120° C. In another aspect, a photothermal therapy agent can be stable no less than about 80° C.

In other embodiments, a photothermal therapy agent disclosed herein can be stable from about 3 days to about 14 days. In an aspect, a photothermal therapy agent can be stable for about 3 days, about 5 days, about 7 days, about 10 days, about 12 days, or about 14 days. In another aspect, a photothermal therapy agent can be stable for about 7 days.

In some embodiments, one or more biofunctionalized nanocomposites as disclosed herein may be an active ingredient in a photothermal therapy agent composition. In an aspect, biofunctionalized nanocomposites disclosed herein may comprise about 0.1 to about 99% by weight in a photothermal therapy agent composition. In other aspects, biofunctionalized nanocomposites disclosed herein may comprise at least 10%, at least 25%, at least 50%, at least 75%, at least 99% by weight in a photothermal therapy agent composition.

In various embodiments, a photothermal therapy agent composition may be in a dosage-form suitable for administration to a subject in need thereof. Non-limiting examples of dosage-forms are tablets, granules, solutions, dispersions, emulsions, capsules, or suspensions.

In various embodiments, a photothermal therapy agent composition may further comprise one or more excipients. In an aspect, excipients suitable for use in a photothermal therapy agent composition disclosed herein may be carriers, diluents, buffers, stabilizers, an anti-oxidant, colorants, other medicinal or pharmaceutical agents, adjuvants, preserving agents, stabilizing agents, wetting agents, emulsifying agents, solution promoters, salts, solubilizers, antifoaming agents, antioxidants, dispersing agents, surfactants, or combinations thereof. In some aspects, a photothermal therapy agent composition may comprise one or more excipients at about 0.1 to about 99% by weight in the photothermal therapy agent composition. In other aspects, excipients may comprise at least 10%, at least 25%, at least 50%, at least 75%, at least 99% by weight in the photothermal therapy agent composition.

### (f) Photothermal Therapy

In some embodiments, biofunctionalized nanocomposites disclosed herein may be used in photothermal therapy. As used herein, “photothermal therapy” is a method of accumulating a material generating heat by absorbing light in a location requiring hyperthermal therapy and irradiating light. Photothermal heating via incident light and PBNPs is termed PBNP-PTT in the present disclosure. Herein, the general principles underlying photothermal treatment (PTT) generally known by those skilled in the art are employed.

In some embodiments photothermal therapy may use a light source to irradiate biofunctionalized nanocomposites disclosed herein. In some embodiments, a biofunctionalized nanocomposite composition disclosed herein may absorb near infrared radiation (NIR) delivered thereto, thus becoming irradiated. Devices and methods for delivering radiation of a particular wavelength include, but not limited to, lasers well-known and standard in the art. In some aspects, the amount of light delivered to a cell via PTT may be determined based on the physical dimensions and thermal characteristics of the tissue to be treated, such that the absorption of said light leads to the desired temperature increase in the tissue. In some embodiments, a method of using biofunctionalized nanocomposites compositions disclosed herein for use in photothermal therapy further comprises calculating output power of the laser based at least in part upon one of heat dissipation and conductivity values within the cell culture or shape factor values of the t cell culture and/or determining time of exposure of the laser. In some embodiments, the light wavelength is in a range of about 600 nm to about 1500 nm (e.g., about 600 nm, about 650 nm, about 700 nm, about 750 nm, about 800 nm, about 850 nm, about 900 nm, about 950 nm, about 1000 nm, about 1500 nm). In preferred embodiments, the light wavelength can range from about 650 nm to about 900 nm.

In some embodiments, biofunctionalized nanocomposites disclosed herein may be irradiated after exposure to a light source for about 4 minutes to about 20 minutes. In other embodiments, biofunctionalized nanocomposites disclosed herein may be irradiated after exposure to a light source for about 4 minutes, about 6 minutes, about 8 minutes, about 10 minutes, about 12 minutes, about 14 minutes, about 16 minutes, about 18 minutes, or about 18 minutes. In a preferred embodiment, biofunctionalized nanocomposites disclosed herein may be irradiated after exposure to NIR for about 10 minutes.

In some embodiments, a method of irradiating biofunctionalized nanocomposites disclosed herein by administering photothermal therapy can result in a thermal interaction at the site of the targeted cell. In some aspects, a thermal interaction may increase temperatures of the targeted cell to at least about 40° C., at least about 41° C., at least about 42° C., at least about 43° C., or at least about 44° C. In other aspects, a thermal interaction may increase temperatures of the targeted cell to stimulate cell and/or tissue death. In yet other aspects, a thermal interaction may increase temperatures of the targeted cell to stimulate cell immune response. In some embodiments, a thermal interaction may increase temperatures of the targeted cell to stimulate cell immune response by at least a 2-fold, at least a 5-fold, at least a 10-fold, at least a 20-fold, at least a 50-fold compared to an untreated cell.

In some embodiments, a method of irradiating biofunctionalized nanocomposites disclosed herein by administering PTT can result a cellular and/or tissue cytotoxic T lymphocyte response. As used herein, a “cytotoxic T lymphocyte response” or “CTL response” refers to an immune response in which cytotoxic T cells are activated by photothermal therapy. A CTL response can include the activation of precursor CTLs as well as differentiated CTLs. In some aspects, a CTL response may include any measurable CTL response for at least one CTL that is specific for an antigen expressed on an autologous tumor cell. In some embodiments, a method of irradiating biofunctionalized nanocomposites disclosed herein by administering PTT may result in an increased the frequency of precursor CTLs specific for tumor antigens compared to an untreated cell. In some embodiments, a method of irradiating biofunctionalized nanocomposites disclosed herein by administering PTT may increase the frequency of precursor CTLs specific for tumor antigens by at least about 2-fold, at least about 5-fold, at least about 10-fold, at least about 20-fold, or at least about 50-fold compared to an untreated cell. In some embodiments, a method of irradiating biofunctionalized nanocomposites disclosed herein by administering PTT may stimulate the frequency of CTLs for a tumor cell compared to an untreated cell. In some embodiments, a method of irradiating biofunctionalized nanocomposites disclosed herein by administering PTT may stimulate the frequency of CTLs for tumor cells by at least about 2-fold, at least about 5-fold, at least about 10-fold, at least about 20-fold, at least about 50-fold compared to an untreated cell. In some embodiments, a method of irradiating biofunctionalized nanocomposites disclosed herein by administering PTT may result in T cell proliferation compared to an untreated cell. In some embodiments, a method of irradiating biofunctionalized nanocomposites disclosed herein by administering PTT may increase T cell proliferation by at least about 50%, at least about 60%, at least about 70%, at least about 80%, or at least about 90%.

In some embodiments, a method of irradiating biofunctionalized nanocomposites disclosed herein by administering PTT may be applied to target cells herein to generate target cell immunogenicity. As used herein, the term “immunogenicity” refers to the ability of cells/tissues to provoke an immune response. In some embodiments, a method of irradiating biofunctionalized nanocomposites disclosed herein by administering PTT may be applied to target cells herein to generate target cell immunogenicity as measured by one or more biochemical correlates of immunogenic cell death (ICD). Examples of biochemical correlates of ICD include, but are not limited to, ATP, calreticulin, HMGB1, and the like. Other cell markers of immunogenicity suitable for use herein can include, but not limited to, co-stimulatory molecule expression, co-inhibitory molecule expression, immune checkpoint expression, MHC expression, and/or antigen release.

### (e) Pharmaceutical Compositions

In certain embodiments, pharmaceutical compositions are provided herein. The pharmaceutical compositions contemplated in the present disclosure can contain a pharmaceutically acceptable carrier combined with one or more biofunctionalized nanocomposites disclosed herein. Pharmaceutically acceptable excipients (carriers) are well known in the art. In some aspects, pharmaceutical compositions herein may comprise at least one pharmaceutically acceptable carrier combined with one or more biofunctionalized nanocomposites comprising a CD137 agonist. In some other aspects, pharmaceutical compositions herein may comprise at least one pharmaceutically acceptable carrier combined with one or more biofunctionalized nanocomposites comprising an anti-CD137 antibody agonist.

In certain embodiments, pharmaceutical compositions disclosed herein can be formulated for parenteral administration by injection. In some aspects, parenteral administration by injection can be by infusion. In some embodiments, pharmaceutical compositions disclosed herein can encompass one or more biofunctionalized nanocomposites comprising a CD137 agonist (e.g., an anti-CD137 antibody agonist) and at least one additional component selected from the group consisting of pharmaceutically acceptable excipients, adjuvants, diluents, preservatives, antibiotics, and combinations thereof.

In some embodiments, pharmaceutical compositions disclosed herein may be formulated in a conventional manner using one or more physiologically acceptable carriers including excipients and auxiliaries which can facilitate processing of active components into preparations which can be used pharmaceutically. In other embodiments, proper formulation of pharmaceutical compositions disclosed herein may be dependent upon the route of administration chosen. In an aspect, any of the well-known techniques, carriers, and excipients may be used as suitable and as understood in the art. A summary of carriers, and excipients suitable for use in immune cell therapy formulations described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety for such disclosure.

“Adjuvants” as used herein are agents that enhance the immune response of an antigen. In some embodiments, one or more adjuvants may be a particulate adjuvant. In some embodiments, one or more adjuvants may be an emulsion. In some embodiments, one or more adjuvants may be a water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil can be used in combination with emulsifiers to form the emulsion. The emulsifiers may be nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E.S.). JohnWiley and Sons, NY, pp51-94 (1995) and Todd et al., Vaccine 15:564-570 (1997). In some embodiments, one or more adjuvants may be a liposome. In some embodiments, one or more adjuvants may be a microsphere of biodegradable polymers. In some embodiments, one or more adjuvants may be an immunomodulator. In some embodiments, an adjuvant system of the present disclosure may be any combination of adjuvants and immunomodulators. Non-limiting examples of immunomodulators comprise monophosphoryl lipid A, bark-saponin Quil A, dsRNA analogues, and N-acetyl muramyl-L-alanyl-D-isoglutamine. Further suitable adjuvant systems useful to the present disclosure include, but are not limited to, the RIBI adjuvant system (Ribi Inc.), Block co-polymer (CytRx, Atlanta GA), SAF-M (Chiron, Emeryville CA), AS15, MF59, Avridine lipid-amine adjuvant, heat-labile enterotoxin from E. coli (recombinant or otherwise), cholera toxin, IMS 1314, GLA-SE, IC31, CAF01, ISCOMs, or muramyl dipeptide among many others.

In some embodiments, pharmaceutical compositions disclosed herein that are formulations for injection may be presented in unit dosage form. In some aspects, a unit dosage form may be in ampoules and or in multi-dose containers. In other aspects, pharmaceutical compositions disclosed herein may be suspensions, solutions or emulsions in oily or aqueous vehicles. In still other aspects, pharmaceutical compositions disclosed herein may contain formulatory agents such as suspending, stabilizing and/or dispersing agents.

In some embodiments, pharmaceutical compositions described herein for parenteral administration can include aqueous and non-aqueous (oily) sterile injection solutions of the compositions which may contain antioxidants, buffers, bacteriostats and solutes which render the formulation isotonic with the blood of the intended recipient; and aqueous and non-aqueous sterile suspensions which may include suspending agents and thickening agents. In some aspects, pharmaceutical compositions described herein may include lipophilic solvents or vehicles. Non-limiting examples of vehicles include fatty oils such as sesame oil, or synthetic fatty acid esters, such as ethyl oleate or triglycerides, or liposomes. In some embodiments, pharmaceutical compositions described herein may be aqueous injection suspensions. In some aspects, pharmaceutical compositions described herein may contain substances which increase the viscosity of the suspension, such as sodium carboxymethyl cellulose, sorbitol, or dextran. In other aspects, pharmaceutical compositions described herein may comprise suitable stabilizers or agents which increase the solubility of the enzymes and fining agents to allow for the preparation of highly concentrated solutions.

### (II) Methods of Use

In certain embodiments, the present disclosure provides for methods of using compositions encompassing a biofunctionalized nanocomposite and/or pharmaceutical compositions encompassing one ore more biofunctionalized nanocomposites as disclosed herein. In some embodiments, methods disclosed herein may be used for treating and/or preventing a cancer in a subject. Some other embodiments of the present disclosure include methods of administering compositions disclosed herein to a subject in need wherein administration treats at least one tumor cell. Still other embodiments of the present disclosure are methods of administering compositions disclosed herein to a subject in need wherein administration prevents metastasis of at least one tumor cell. Other embodiments of the present disclosure are methods of administering compositions disclosed herein to a subject in need wherein administration treats and/or prevents cancer.

In certain embodiments, compositions disclosed herein can be administered to a subject in need thereof. A suitable subject includes a mammal, a human, a livestock animal, a companion animal, a lab animal, or a zoological animal. In some embodiments, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet other embodiments, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In certain embodiments, a subject in need may be suspected of having a cancer and/or be at risk for cancer. In certain embodiments, a subject in need may have been diagnosed with cancer. In some embodiments, the subject may have cancer at any stage of disease progression. In some aspects, a subject may have Stage I-IV cancer. In another embodiment, a subject may have Stage I-III cancer. In some other aspects, a subject may have Stage I-II cancer. In some aspects, a subject may have Stage I cancer. In some other aspects, the cancer may be a primary cancer, a metastases, or primary cancer and metastases. In some aspects, the cancer may be a solid cancer. In some other aspects, the cancer may be a liquid cancer. Non-limiting examples of cancer types include carcinomas, sarcomas, lymphomas, leukemias, myelomas and mixed types (e.g., blastomas).

In some embodiments, a subject in need thereof may have been diagnosed with a cancer. By example, but not limited to, a subject may have been diagnosed with nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, tonsil cancer, or a combination thereof.

In certain embodiments, compositions disclosed herein can be administered to a subject according to an intravenous, intraperitoneal, intradermal, subcutaneous, intrathecal, intracerebral, peri-tumoral, and/or intra-tumoral route of administration. In some embodiments, compositions disclosed herein may be administered by parenteral administration. As used herein, “by parenteral administration” refers to administration of immune cell therapy compositions disclosed herein via a route other than through the digestive tract. In some embodiments, compositions disclosed herein may be administered by parenteral injection. In some aspects, administration of the disclosed compositions by parenteral injection may be by subcutaneous, intramuscular, intravenous, intraperitoneal, intracardiac, intraarticular, or intracavernous injection. In other aspects, administration of the disclosed compositions by parenteral injection may be by slow or bolus methods as known in the field. In some embodiments, the route of administration by parenteral injection can be determined by the target location. In some aspects, compositions disclosed herein may be administered to a solid tumor.

In certain embodiments, the dosages of compositions disclosed herein to be administered are not particularly limited and may be appropriately chosen depending on conditions such as a purpose of preventive and/or therapeutic treatment, a type of a disease, the body weight or age of a subject, severity of a disease and the like. In certain embodiments, the amount and/or frequency of compositions disclosed herein administered can be adjusted based upon factors such as the particular compound, disease condition and its severity, according to the particular circumstances surrounding the case, including, e.g., the route of administration, the condition being treated, the target area being treated, and the subject or host being treated. In certain embodiments, administration of a dose of a composition disclosed herein may comprise a therapeutically effective amount of the composition disclosed herein. As used herein, the term “therapeutically effective” refers to an amount of administered composition that reduces the amount of viable tumor cells, reduces the rate of metastasis, impairs tumor growth, shrinks tumor size, decreases the presence of at least one tumor marker, improve cancer life expectancy, or a combination thereof. A therapeutically effective amount of a composition disclosed herein to be delivered to a subject may be an amount that does not result in undesirable systemic side effects.

In certain embodiments, compositions administered as disclosed herein may comprise about 5% to about 95%, about 15% to about 85%, or about 25% to about 75% total biofunctionalized nanocomposite by total weight of the composition. In some embodiments, compositions administered as disclosed herein may comprise about 5% to about 95%, about 15% to about 85%, or about 25% to about 75% total photothermal therapy agent by total weight of the composition. In still some other embodiments, photothermal therapy agent compositions administered as disclosed herein may comprise about 5% to about 95%, about 15% to about 85%, or about 25% to about 75% total biofunctionalized nanocomposite by total weight of the composition. In some embodiments, a composition disclosed herein may be administered to a subject in need thereof once. In some embodiments, a composition disclosed herein may be administered to a subject in need thereof more than once. In some embodiments, a first administration of a composition disclosed herein may be followed by a second administration of a composition disclosed herein. In some embodiments, a first administration of a composition disclosed herein may be followed by a second and third administration of a composition disclosed herein. In some embodiments, a first administration of a composition disclosed herein may be followed by a second, third, and fourth administration of a composition disclosed herein. In some embodiments, a first administration of a composition disclosed herein may be followed by a second, third, fourth, and fifth administration of a composition disclosed herein.

The number of times a composition may be administered to a subject in need thereof can depend on the discretion of a medical professional, the disorder, the severity of the disorder, and the subject's response to the formulation. In some embodiments, a composition disclosed herein may be administered once to a subject in need thereof with a mild acute condition, for example a subject with Stage I-II cancer. In some embodiments, a composition disclosed herein may be administered more than once to a subject in need thereof with a moderate or severe acute condition, for example a subject with Stage I-IV cancer. In some embodiments, a composition disclosed herein may be administered continuously; alternatively, the dose of drug being administered may be temporarily reduced or temporarily suspended for a certain length of time (i.e., a “drug holiday”). In some aspects, the length of the drug holiday can vary between 2 days and 1 year, including by way of example only, 2 days, 1 week, 1 month, 6 months, and 1 year. In some other aspects, dose reduction during a drug holiday may be from 10%-100%, including by way of example only 10%, 25%, 50%, 75%, and 100%.

In some embodiments, the desired daily dose of compositions disclosed herein may be presented in a single dose or as divided doses administered simultaneously (or over a short period of time) or at ap-propriate intervals. In some embodiments, administration of a composition disclosed herein may be administered to a subject about once a day, about twice a day, about three times a day. In other embodiments, administration of a composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter. In a preferred embodiment, administration of a composition dis-closed herein may be administered to a subject once a day for at about 4 weeks.

In some embodiments, administration of a composition disclosed herein may be administered to a subject at least once a week, at least once a week for about 2 weeks, at least once a week for about 3 weeks, at least once a week for about 4 weeks, at least once a week for about 8 weeks, at least once a week for about 12 weeks, at least once a week for about 16 weeks, at least once a week for about 24 weeks, at least once a week for about 52 weeks and thereafter. In some aspects, administration of a composition disclosed herein may be administered to a subject once a week for at about 12 weeks.

In certain embodiments, the present disclosure provides methods for treating cancer in a subject herein. In some embodiments, methods herein may be used for treating a solid tumor in a subject. Non-limiting examples of solid tumors that may be treated by the methods herein may include pancreatic ductal adenocarcinoma (PDA), colorectal cancer (CRC), melanoma, cholangiocarcinoma, breast cancer, small cell and non-small cell lung cancer, upper and lower gastrointestinal malignancies, gastric cancer, squamous cell head and neck cancer, genitourinary cancer, hepatocellular carcinoma, ovarian cancer, sarcomas, mesothelioma, glioblastoma, esophageal cancer, bladder cancer, urothelial cancer, renal cancer, cervical and/or endometrial cancer. In some embodiments, the cancer that may be treated by the methods herein may be adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, cholangiocarcinoma, cholangiosarcoma, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, genitourinary cancers, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer (for example, non-small cell lung cancer, NSCLC, and small cell lung cancer, SCLC), lymphoma (e.g., AIDS-related lymphoma, Burkitt lymphoma, cutaneous T cell lymphoma, Hogkin lymphoma, Non-Hogkin lymphoma, primary central nervous system lymphoma), malignant mesothelioma, multiple myeloma, myelodysplastic syndrome, nasal cavity cancer, paranasal sinus cancer, pancreatic duct adenocarcinoma (PDA) nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), squamous cell head and neck cancer, small intestine cancer, stomach cancer, teratoid tumor, testicular cancer, throat cancer, thymus cancer, thyroid cancer, unusual childhood cancers, upper and lower gastrointestinal malignancies (including, but not limited to, esophageal, gastric, and hepatobiliary cancer), urethral cancer, uterine cancer, uterine sarcoma, vaginal cancer, vulvar cancer, Waldenstrom macroglobulinemia, and Wilms tumor. In some embodiments, the cancer may be selected from hematological malignancies including acute lymphoblastic leukemia, chronic lymphocytic leukemia, lymphomas, multiple myeloma, acute myelogenous leukemia, chronic myelogenous leukemia, myelodysplastic syndromes and the myeloproliferative neoplasms, such as essential thrombocythemia, polycythemia vera, myelofibrosis, gallbladder cancer (adenocarcinomas or squamous cell carcinoma), or any combination thereof.

In certain embodiments, the present disclosure provides methods for treating one ore more symptoms associated with a cancer in a subject herein. In some embodiments, the symptom(s) associated with the disease (e.g., cancer) may include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, problems with memory and concentration, or any combination thereof.

A subject having any of the above noted cancers can be identified by routine medical examination, e.g., laboratory tests, organ functional tests, genetic tests, interventional procedure (biopsy, surgery) and/or any relevant imaging modalities. In some embodiments, a subject to be treated by methods described herein may be a human cancer patient who has undergone or is subjecting to an anti-cancer therapy, for example, chemotherapy, radiotherapy, immunotherapy, or surgery. In some embodiments, subjects may have received prior immunomodulatory anti-tumor agents. Non-limiting examples of such immunomodulatory agents include, but are not limited to as anti-PD1, anti-PD-L1, anti-CTLA-4, anti-OX40, and the like. In some embodiments, a subject herein can show disease progression through the treatment. In other embodiments, a subject herein may be resistant to the treatment (either de novo or acquired). In some embodiments, such a subject may demonstrate as having one or more advanced malignancies (e.g., inoperable or metastatic). Alternatively or in addition, in some embodiments, a subject herein may have no standard therapeutic options available or ineligible for standard treatment options, which refer to therapies commonly used in clinical settings for treating the corresponding solid tumor (i.e., a subject having a terminal cancer). Alternatively or in addition, in some embodiments, a subject herein may be a human patient having a refractory disease. As used herein, “refractory” refers to cancer and/or tumor that does not respond to and/or becomes resistant to a treatment. In some instances, a subject herein may be a human patient having a relapsed disease. As used herein, “relapsed” or “relapses” refers to a tumor that returns or progresses following a period of improvement (e.g., a partial or complete response) with treatment.

Embodiments of the instant disclosure also provide methods and compositions for treating cancers, tumors, or any combination thereof resistant to or suspected of becoming resistant to one or more anticancer drugs/therapies. As used herein an “anticancer drug” refers to any drug that for the treatment of malignant, or cancerous, disease. Anticancer therapy refers to a treatment regime for the treatment of malignant, or cancerous, disease such as administration of an anticancer drug, radiation, surgical methods, and the like. In certain embodiments, methods disclosed herein may be used treating cancers, tumors, or any combination thereof resistant to or suspected of becoming resistant to one or more ICIs. In certain embodiments, methods disclosed may prevent and/or attenuate the growth of tumors resistant to one or more immunotherapy drugs, particularly immune checkpoint inhibitors. Immunotherapy drugs called immune checkpoint inhibitors work by blocking checkpoint proteins from binding with their partner proteins. This prevents the “off” signal from being sent, allowing the T cells to kill cancer cells. As an example, but not limited to, immune checkpoint inhibitors may block immune checkpoint proteins cytotoxic T lymphocyte-associated molecule-4 (CTLA-4), programmed cell death receptor-1 (PD-1), and programmed cell death receptor-1 ligand (PD-L1), and the like. Non-limiting examples of immune checkpoint inhibitors include ipilimumab, pembrolizumab, nivolumab, cemiplimab, dostarlimab, atezolizumab, avelumab, durvalumab, tremelimumab, tebotelimab, MBG453, MGC018, AB928, CPI-006, monalizumab, COM701, CM24, NEO-201, defactinib, PF-04136309, MSC-1, TTI-662, lacnotuzumab, emactuzumab, pexidartinib, canakinumab, pepinemab, trebananib, FP-1305, enapotamab vedotin, bavituximab.

In certain embodiments, a tumor to be treated by compositions and methods disclosed herein can be a solid tumor. In some embodiments disclosed herein, compositions and methods disclosed herein are designed to re-sensitize or sensitize a tumor in a subject to anticancer drug (e.g., one or more ICIs) to reduce costs, improve outcome and reduce or eliminate patient exposure to an anticancer therapy without significant effect.

In some embodiments, a subject can have an ICI resistant tumor or be suspected of developing such a tumor where additional agents can be administered to re-sensitize or sensitize a tumor in a subject where the tumor includes a solid tumor. In some embodiments, a solid tumor can be an abnormal mass of tissue that is devoid of cysts or liquid regions within the tumor. In some embodiments, solid tumors can be benign (not progressed to a cancer), a malignant or metastatic tumor. In some embodiments, a solid tumor herein can be a malignant cancer that has metastasized. In other embodiments, solid tumors contemplated herein can include, but are not limited to, sarcomas, carcinomas, lymphomas, gliomas or a combinational thereof. In accordance with some embodiments herein, tumors resistant to platinum-based chemotherapy can include, but are not limited to, a testicular tumor, ovarian tumor, cervical tumor, a kidney tumor, bladder tumor, head-and-neck tumor, liver tumor, stomach tumor, lung tumor, endometrial tumor, esophagcal tumor, breast tumor, cervical tumor, central nervous system tumor, germ cell tumor, prostate tumor, Hodgkin's lymphoma, non-Hodgkin's lymphoma, neuroblastoma, sarcoma, multiple myeloma, melanoma, mesothelioma, osteogenic sarcoma or a combination thereof. In some embodiments, a targeted tumor contemplated herein can include a solid tumor such as ovarian tumors, breast tumors, or any combination thereof.

In certain embodiments, the nanoparticles comprising Prussian blue materials as disclosed herein can be administered concurrently with the one or more CD137 agonists by the same or different modes of administration. In some embodiments, the one or more nanoparticles comprising Prussian blue materials as disclosed herein can be administered before, during or after the one or more CD137 agonists. In certain embodiments, photothermal therapy may be administered following concurrent administration of the nanoparticles comprising Prussian blue materials and one or more CD137 agonists. In certain embodiments, photothermal therapy may be administered following administration of the nanoparticles comprising Prussian blue materials before, during or after administration of one or more CD137 agonists. In certain embodiments, the nanoparticles comprising Prussian blue materials as disclosed herein can be administered after photothermal therapy to boost immune response in the subject.

In some embodiments, a subject treated with any of the methods herein can have completed an additional therapeutic regimen, be receiving an additional therapeutic regimen, or can receive an additional therapeutic regimen following treatment according to the methods herein. In some embodiments, an additional therapeutic regimen for use herein can include administering a chemotherapeutic agent. In some embodiments, a chemotherapeutic agent can be a cell cycle inhibitor. As used herein “cell cycle inhibitor” can include a chemotherapeutic agent that inhibits or prevents the division and/or replication of cells. In some embodiments, a cell cycle inhibitor can include a chemotherapeutic agent such as doxorubicin, melphlan, roscovitine, mitomycin C, hydroxyurea, 5-fluorouracil, cisplatin, ara-C, etoposide, gemcitabine, bortezomib, sunitinib, sorafenib, sodium valproate, a HDAC inhibitor, or dacarbazine. More examples of additional chemotherapeutic agents include but are not limited to HDAC inhibitors such as FR01228, trichostatin A, SAHA and/or PDX101. In some embodiments, the cell cycle inhibitor is a DNA synthesis inhibitor. As used herein, a “DNA synthesis inhibitor” can include a chemotherapeutic agent that inhibits or prevents the synthesis of DNA by a cancer cell. Examples of DNA synthesis inhibitors include but are not limited to AraC (cytarabine), 6-mercaptopurine, 6-thioguanine, 5-fluorouracil, capecitabine, floxuridine, gemcitabine, decitabine, vidaza, fludarabine, nelarabine, cladribine, clofarabine, pentostatin, thiarabine, troxacitabine, sapacitabine or forodesine. More examples of additional chemotherapeutic agents include, but are not limited to, FLT3 inhibitors such as Semexanib (SCT5416), Sunitinib (SU 11248), Midostaurin (PKC412), Lestautinib (CEP-701), Tandutinib (MLN518), CHIR-258, Sorafenib (BAY-43-9006) and/or KW-2449. More non-limiting examples of additional chemotherapeutic agents include farnesyltransferase inhibitors such as tipifarnib (Rl 15777, Zarnestra), lonafarnib (SCH66336, Sarasar™) and/or BMS-214662. More examples of additional chemotherapeutic agents include, but are not limited to, topoisomerase II inhibitors such as the epipodophyllotoxins etoposide, teniposide, anthracyclines doxorubicin and/or 4-epi-doxorubicin. More non-limiting examples of additional chemotherapeutic agents include P-glycoprotein modulators such as zosuquidar trihydrochloride (Z.3HCL), vanadate, or verapamil. More non-limiting examples of additional chemotherapeutic agents include hypomethylating agents such as 5-aza-cytidine or 2′ deoxyazacitidine.

In some embodiments, an additional therapeutic regimen for use herein can include administering one or more immunomodulatory agents. Non-limiting examples of such immunomodulatory agents can include on or more monoclonal antibody (mAb) therapies. In some embodiments, a mAb therapy can target HER2; EGFR; VEGFR; VEGF; CD-20; CD-22; CD-52; CD-33; CD-30; CD19/CD3; CD38; CTLA-4; PD-1; PD-L1; RANKL; GD2; PDGFR; SLAMF7, or any combination thereof. Non-limiting examples of mAb therapies suitable for use herein can include adotrastuzumab, trastuzumab, pertuzumab, cetuximab, panitumumab, necitumumab, ramucirumab, bevacizumab, rituximab, ofatumumab, ibritumomab, tositumomab, obinutuzumab, inotuzumab, alemtuzumab, gemtuzumab, brentuximab, blinatumomab, daratumumab, ipilimumab, nivolumab, atezolizumab, avelumab, cemiplimab, pembrolizumab, durvalumab, denosumab, dinutuximab, olaratumab, clotuzumab, and the like.

In some embodiments, an additional therapeutic regimen for use herein can include administering one or more small molecules. Non-limiting examples of such small molecules can include imatinib, dasatinib, nilotinib, bosutinib, regorafenib, ponatinib, sunitinib, sorafenib, erdafitinib, lenvatinib, pazopanib, afatinib, gefitinib, osimertinib, vandetanib, erlotinib, lapatinib, dacomitinib, neratinib, ribociclib, abemaciclib, palbociclib, cabozantinib, crizotinib, axitinib, alectinib, vemurafenib, encorafenib, dabrafenib, olaparib, rucaparib, talazoparib, niraparib, larotrectinib, entrectinib, lorlatinib, ibrutinib, cobimctinib, binimctinib, trametinib, brigatinib, cgilteritinib, ceritinib, ivosidenib, carfilzomib, marizomib, alpelisib, duvelisib, copanlisib, and the like.

In certain embodiments, methods of treatment disclosed herein (e.g., photothermal therapy) can impair tumor growth compared to tumor growth in an untreated subject with identical disease condition and predicted outcome. In some embodiments, tumor growth can be stopped following treatments (e.g., photothermal therapy) according to the methods disclosed herein. In some embodiments, tumor growth can be impaired at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other words, tumors in a subject treated according to the methods disclosed herein may grow at least 5% less (or more as described above) when compared to an untreated subject with identical disease condition and predicted outcome. In some embodiments, tumor growth can be impaired at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, tumor growth can be impaired at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated subject with identical disease condition and predicted outcome.

In some embodiments, treatment of tumors according to the methods disclosed herein (e.g., photothermal therapy) can result in a shrinking of a tumor in comparison to the starting size of the tumor. In some embodiments, tumor shrinking is at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the tumor is completely gone after treatment) compared to the starting size of the tumor.

In some embodiments, treatments administered according to the methods disclosed herein (e.g., photothermal therapy) can improve patient life expectancy compared to the cancer life expectancy of an untreated subject with identical disease condition and predicted outcome. As used herein, “patient life expectancy” is defined as the time at which 50 percent of subjects are alive and 50 percent have passed away. In some embodiments, patient life expectancy can be indefinite following treatment according to the methods disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

In some embodiments, a subject to be treated by any of the methods herein (e.g., photothermal therapy) can present with one or more cancerous solid tumors, metastatic nodes, of a combination thereof. In some embodiments, a subject herein can have a cancerous tumor cell source that can be less than about 0.2 cm³ to at least about 20 cm³ or greater, at least about 2 cm³ to at least about 18 cm³ or greater, at least about 3 cm³ to at least about 15 cm³ or greater, at least about 4 cm³ to at least about 12 cm³ or greater, at least about 5 cm³ to at least about 10 cm³ or greater, or at least about 6 cm³ to at least about 8 cm³ or greater.

In some embodiments, any of the methods disclosed herein (e.g., photothermal therapy) can further include monitoring occurrence of one or more adverse effects in the subject. Exemplary adverse effects include, but are not limited to, hepatic impairment, hematologic toxicity, neurologic toxicity, cutaneous toxicity, gastrointestinal toxicity, or a combination thereof. When one or more adverse effects are observed, the method disclosed herein can further include reducing or increasing the dose of the compositions disclosed herein, the dose of one or more anticancer drugs (e.g., chemotherapeutics, small molecules, mAbs) or both depending on the adverse effect or effects in the subject.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Introduction to Examples 1-7

Despite the promise of immunotherapy such as the immune checkpoint inhibitors (ICIs) anti-PD-1 and anti-CTLA-4 for advanced melanoma, only 26%-52% of patients respond, and many experience grade III/IV immune-related adverse events. Motivated by the need for an effective therapy for patients non-responsive to clinically approved ICIs, the present disclosure provides, in part, a novel nanoimmunotherapy that combines locally administered Prussian blue nanoparticle-based photothermal therapy (PBNP-PTT) with systemically administered agonistic anti-CD137 monoclonal antibody therapy (aCD137).

### Example 1

In an exemplary method, the synthesis of αCD137-PBNPs was optimized. In brief, the optimum ratio of PBNP to αCD-137 for charge-charge binding was determined. PBNPs naturally have a charge of −30 mV and αCD-137 has a net positive charge in a DI H₂O and PBNP solution. Assays were performed in order to identify a ratio that completely binds all αCD-137 with none left behind after synthesis. Prussian Blue has a quenching effect on fluorescently tagged antibodies. In order to evaluate attachment of αCD-137 to the nanoparticles, a BCA assay was used on the supernatant that was collected after centrifugation of the nanoparticles during synthesis. Attachment was assessed by measuring the size and charge of the nanoparticles.

αCD137-PBNPs synthesis. For αCD137-PBNPs synthesis, each of the varying amounts of αCD-137 was contacted with 250 μg PBNP for 1 hour in a 1.7 ml Eppendorf tube. The solution was then spun down at 15000 RPM. The supernatant was then separated and analyzed for αCD-137 via BCA Assay. The pellet was resuspended in DI H₂O and analyzed via dynamic light scattering and zeta potential with a Malvern Zeta Sizer. Nanoparticles were allowed to sit before measuring the size and zeta potential every day after synthesis until a notable change in size was found.

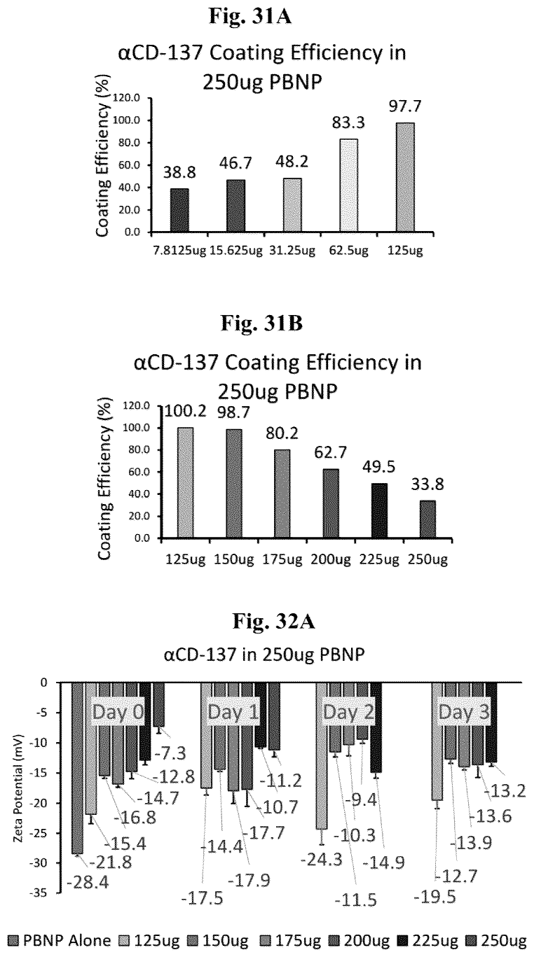

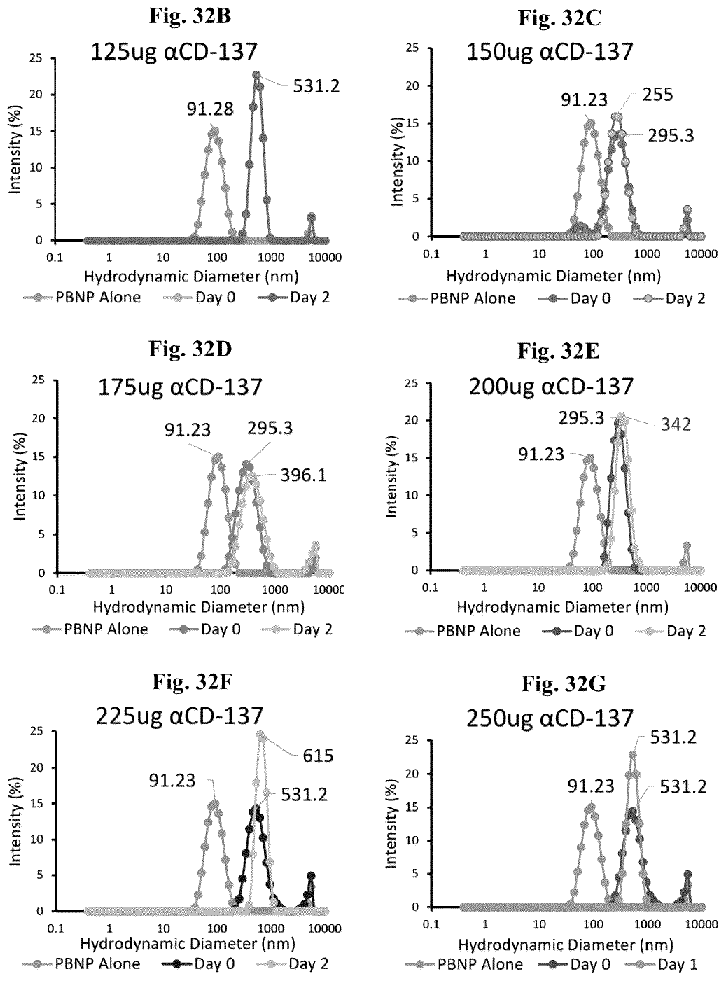

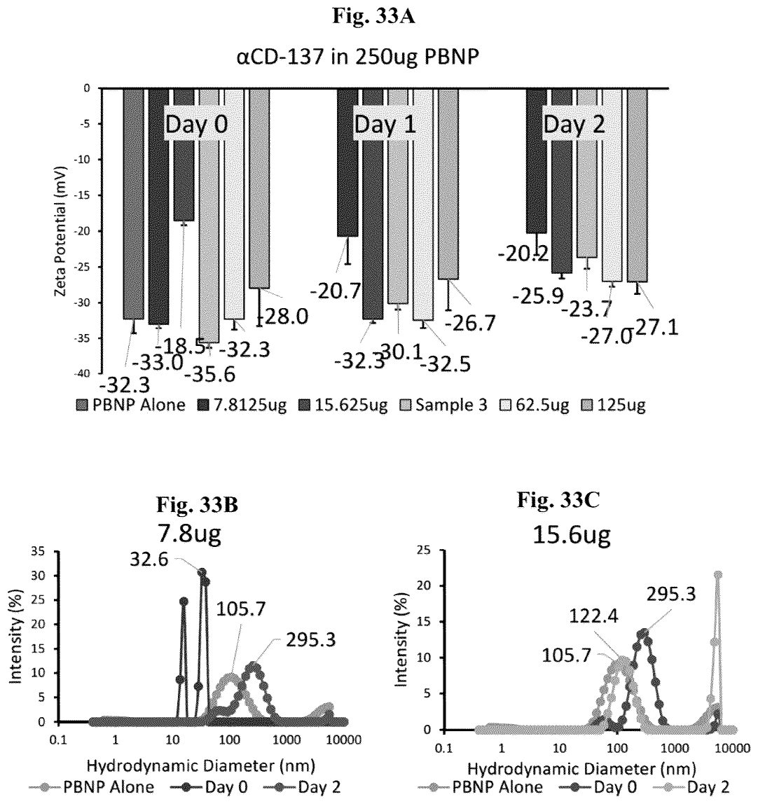

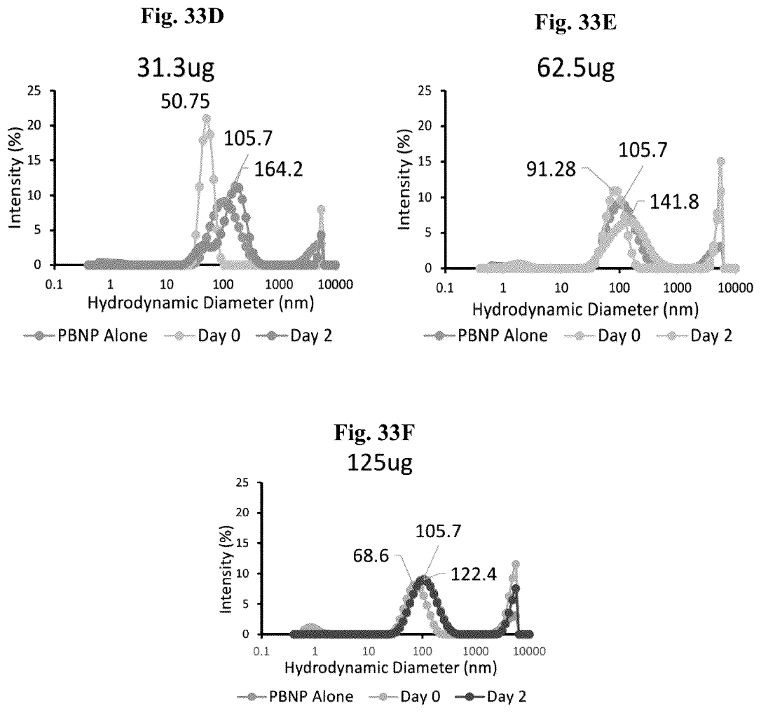

Mass Ratios of PBNP to αCD-137. In order to determine the optimum concentration of αCD-137 to PBNP, different amounts of αCD-137 were chosen between the mass αCD-137: mass PBNP ratios of 1:32 and 1:1 with the ratio of 1:2 serving as the middle point. The data appeared this way because the decreasing amounts of αCD-137 (FIGS. 33A-33F) were performed as a separate experiment from the increasing amounts of αCD-137 (FIGS. 32A-32G). FIGS. 31A-31B shows the coating efficiency of αCD-137 onto PBNPs as determined by BCA Assay of the supernatant following centrifugation. It was found that 125 μg αCD-137:250 μg PBNP yielded no traceable of amounts of αCD-137 after synthesis. It was also found that particles tend to lose stability and aggregate after 2 days. FIGS. 32A-32G and 33A-33F show sizes and up until aggregation. Data showed that the 2 μg/ml PBNP to 1 μg/ml αCD-137 ratio is the optimal ratio for synthesis of αCD137-PBNPs.

Synthesis of Bioconjugated αCD137-PBNP. Covalent synthesis of the biohybrid (αCD137-PBNP) nanoparticle was carried out using EDC (1-ethyl-3-(-3-dimethylaminopropyl) carbodiimide hydrochloride) chemistry. To begin, 21.5 μL of prepared citrate-coated PBNP (23.21 mg/mL) was combined with 100 μL of EDC solution (2.2 mg/mL; Thermo Fisher, Waltham, MA, USA; Cat. No. 22980) and 100 μL of Sulfo-NHS solution (8.0 mg/mL; Millipore Sigma, Darmstadt, Germany; Cat. No. 56485) in 1 mL of MES buffer (0.1 M MES, 0.5 M NaCl, pH=5; ACROS Organics, Geel, Belgium; Cat. No. 397350250). The first crosslinking reaction occurred for 15 minutes at room temperature (RT; 23° C.±5° C.) and stopped via addition of 100 μL of 2-mercaptoethanol (8.9 mg/mL; Thermo Fisher, Waltham, MA, USA; Cat. No. 21985023) inside a chemical fume hood. Citrate-coated PBNP crosslinked to Sulfo-NHS were then centrifuged at 22,000 g×30 minutes using a table-top microcentrifuge unit at RT. Particles were collected, suspended in 1 mL MES buffer, and sonicated at 40% amp for 30 seconds to achieve a homogeneous colloidal solution. aCD137 antibody was then added to a final concentration of 0.25 μg/mL, corresponding to a 1:2000 mass-to-mass ratio of aCD137:PBNP. The samples were then contacted at RT for 3 hours on an orbital shaker, after which 100 μL of 0.1 M hydroxylamine (Thermo Fisher, Waltham, MA, USA; Cat. No. 26103) was added to quench any remaining primary amine sites. aCD137-PBNPs were again centrifuged at 22,000 g×30 minutes at RT, collected, resuspended in 1 mL H₂O, and sonicated to again achieve a homogeneous colloidal solution. This process was repeated twice for a total of three washes, after which particles were resuspended in the desired buffer for use.

### Example 2

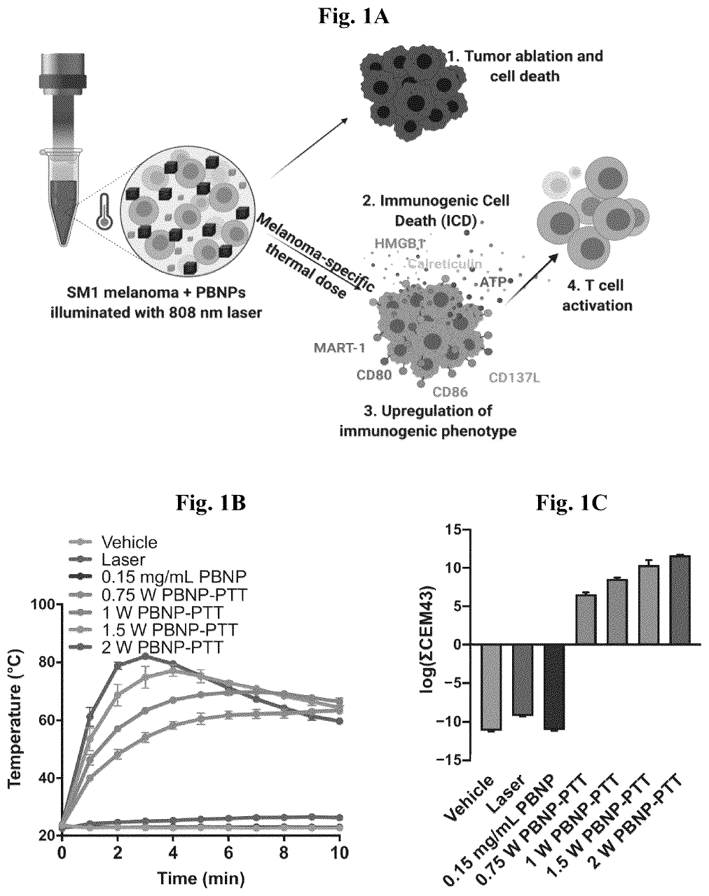

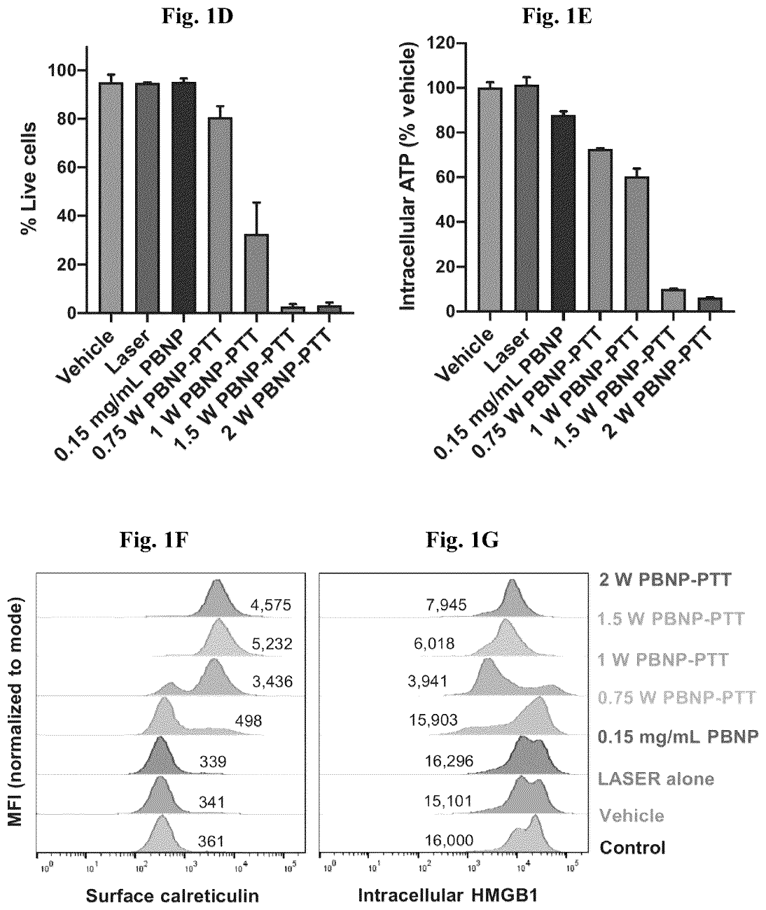

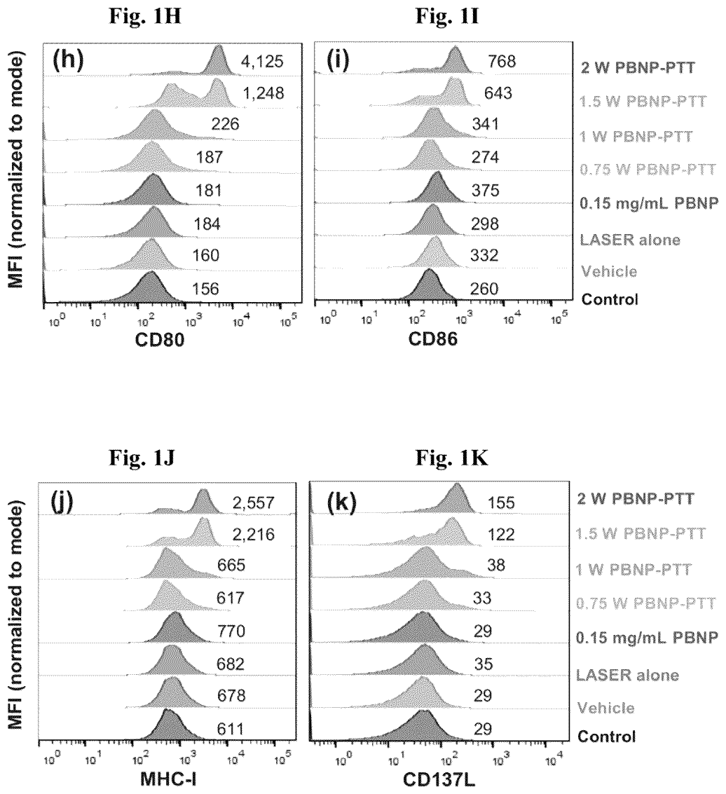

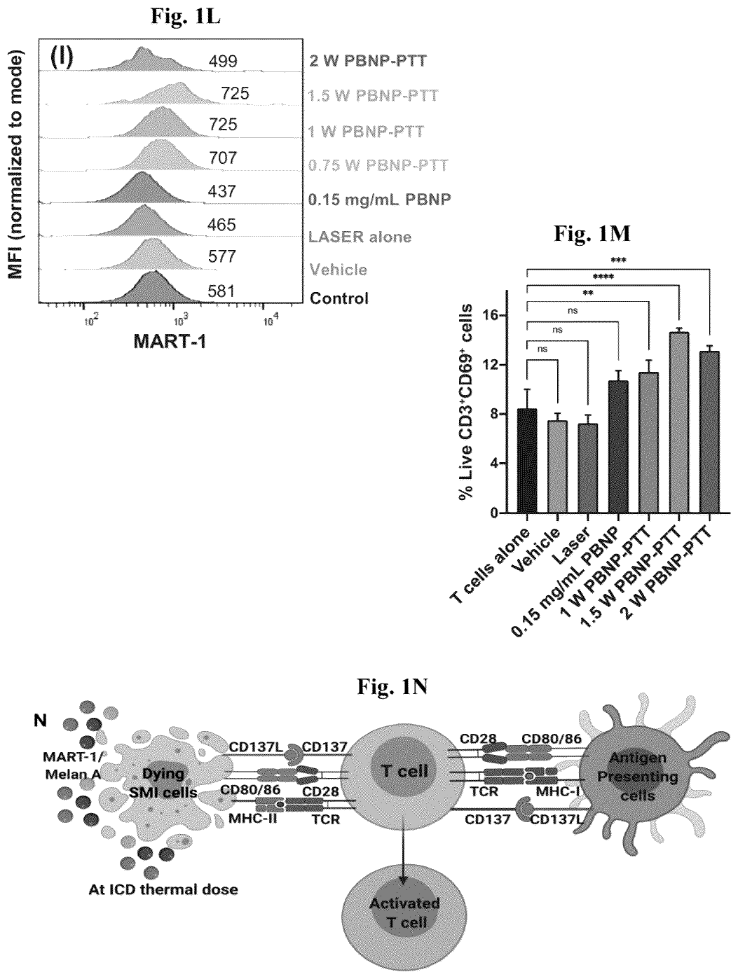

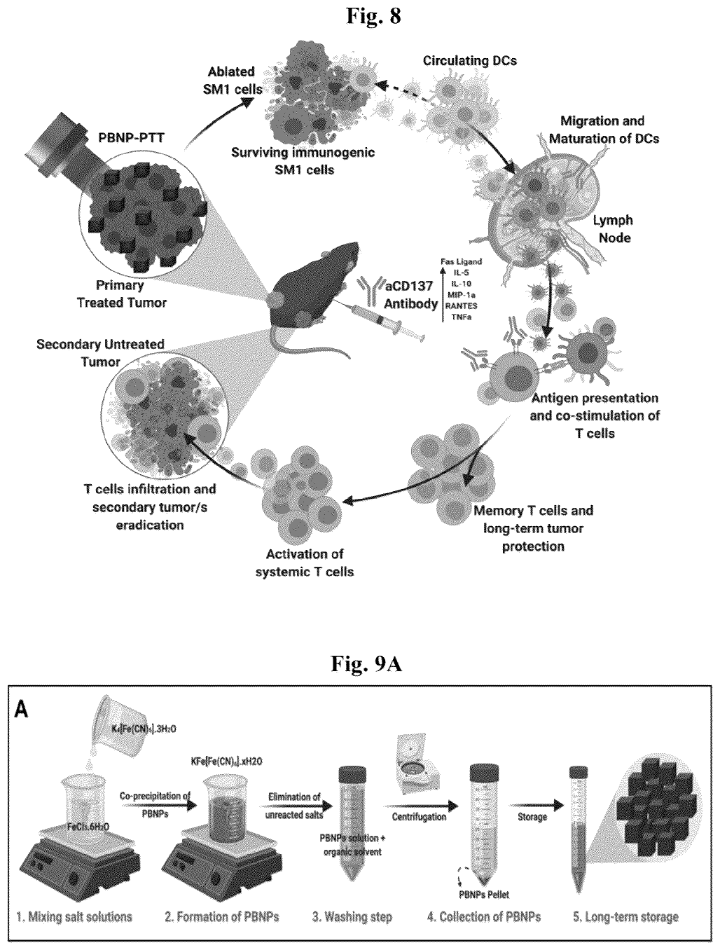

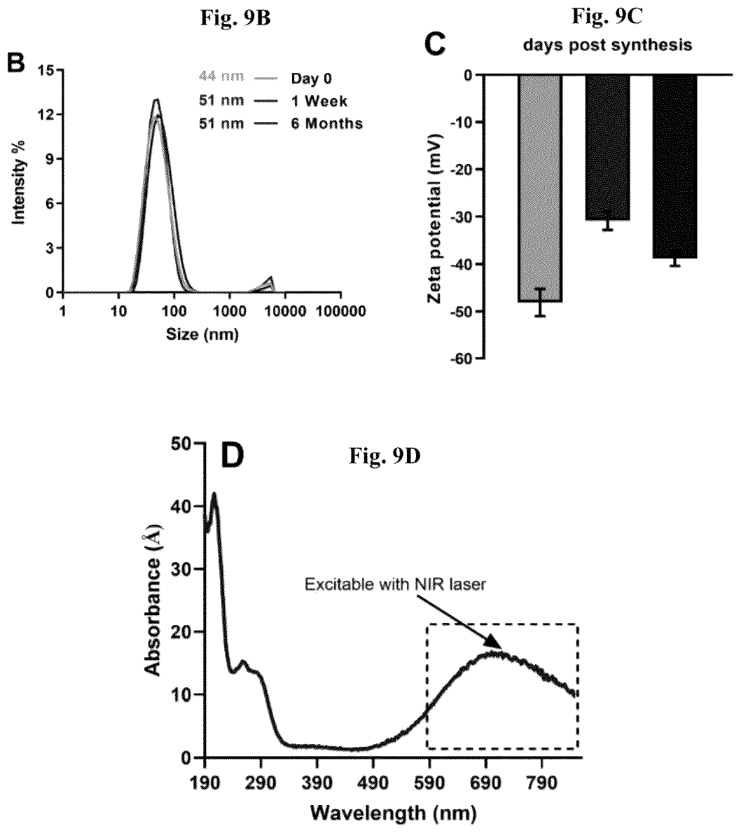

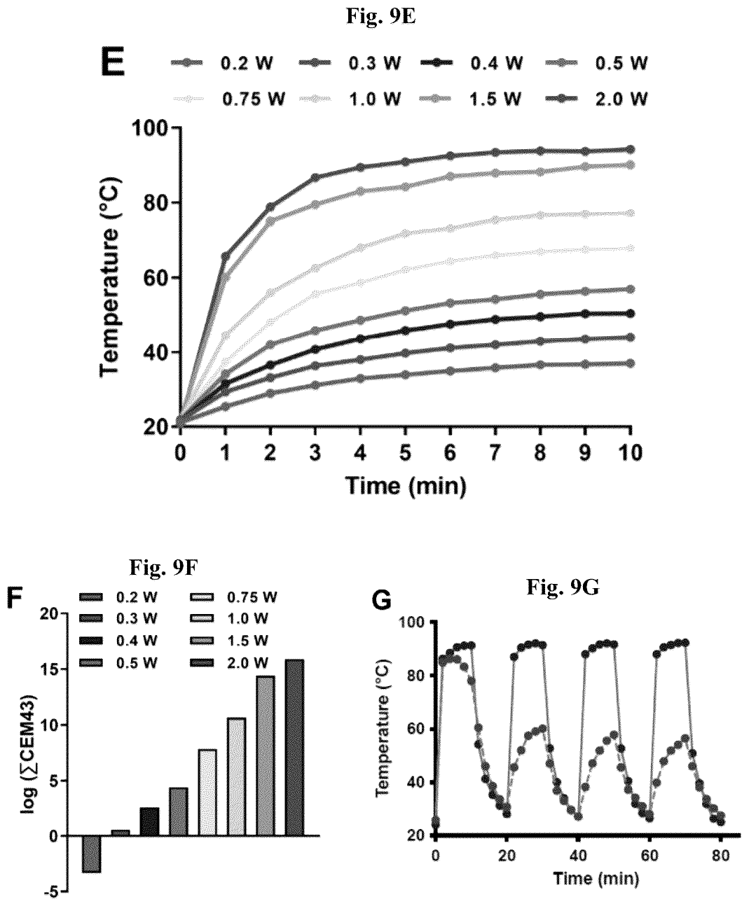

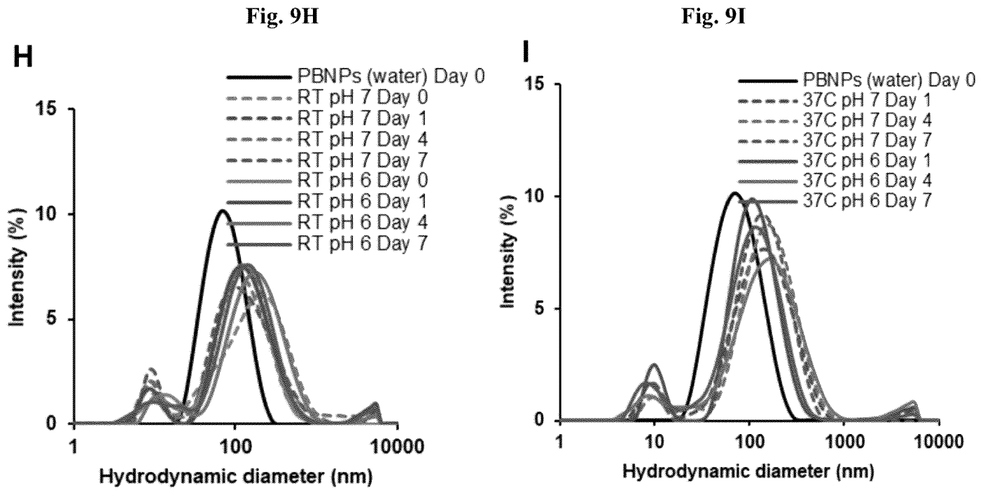

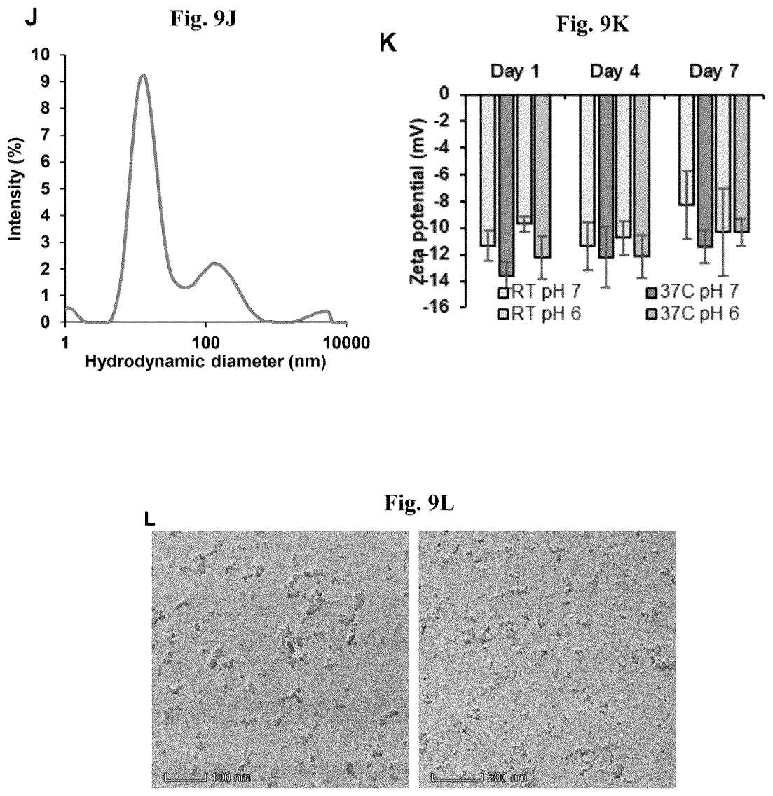

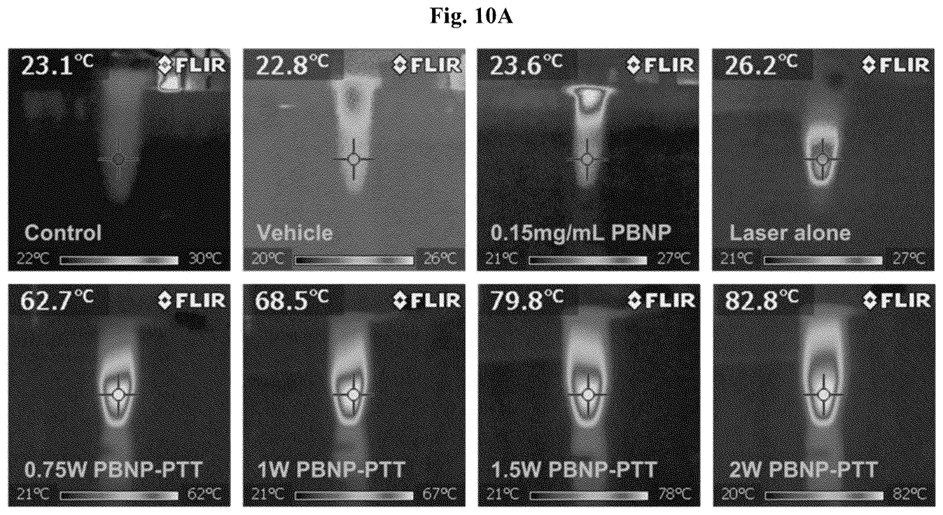

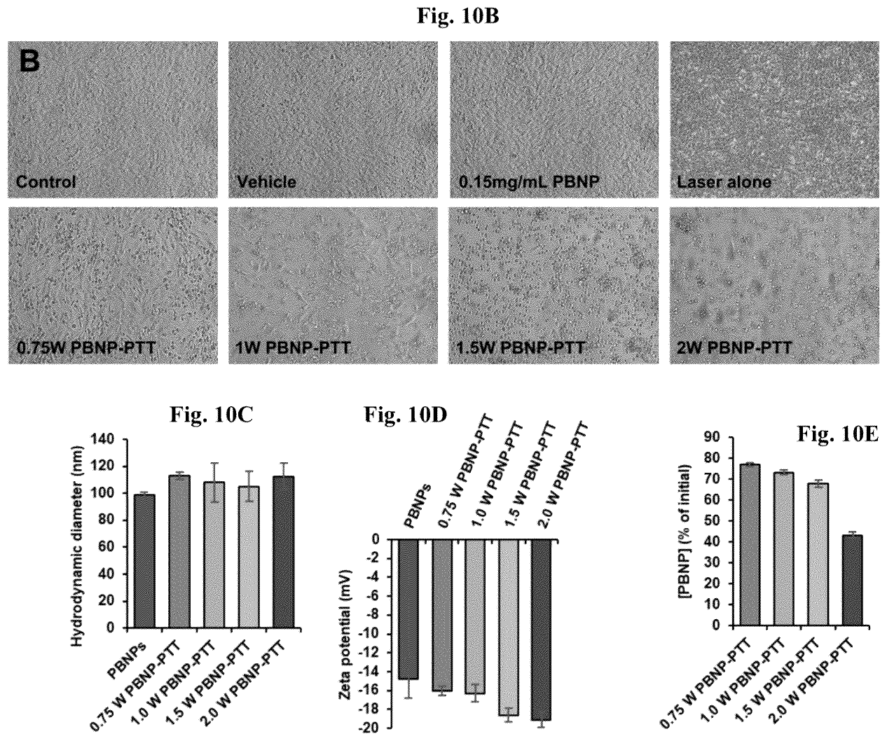

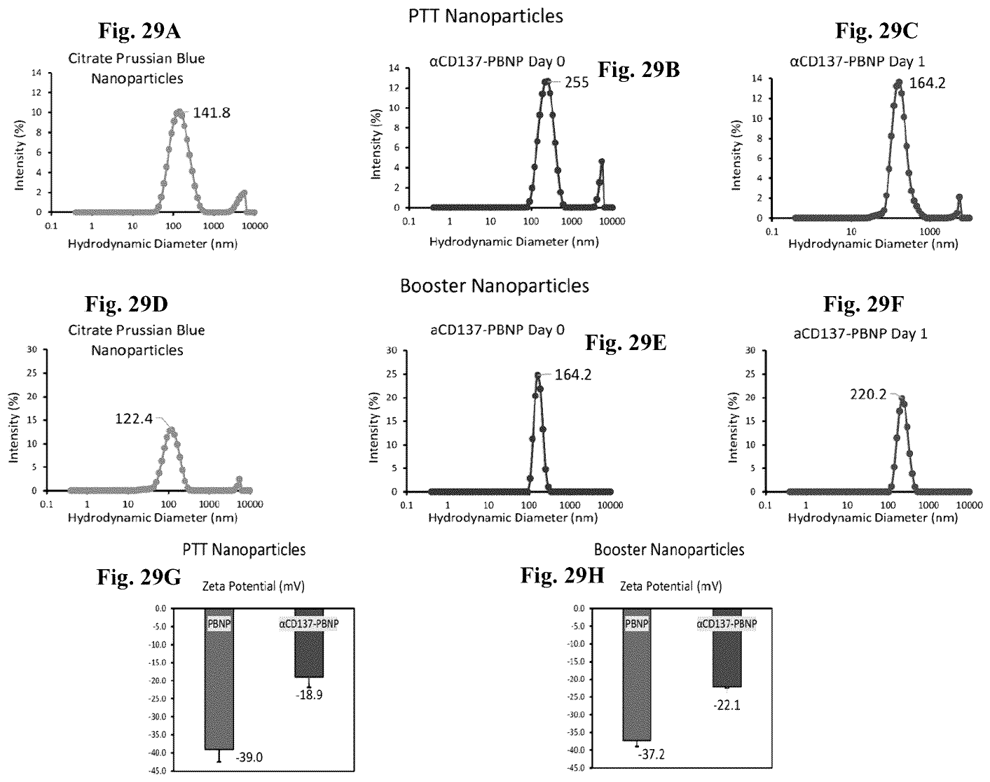

In an exemplary method, PBNP-PTT increased the immunogenicity of melanoma cells, which directly activated T cells in a thermal dose-dependent manner in vitro. In brief, to uncover the immunogenic effects of PBNP-PTT on melanoma cells, metrics for measuring immunogenicity in vitro were first established. Specifically, melanoma cell death, the upregulation of markers defining ICD, the induction of molecular patterns associated with immune cell engagement, and the direct activation of T cells were measured after co-culturing the cells with PBNP-PTT-treated melanoma cells (FIG. 1A). To perform these analyses, PBNPs were synthesized and characterized for uniform size distributions, stability, and photothermal heating properties (FIGS. 9A-9L and FIGS. 29A-29H). Using a constant PBNP concentration (0.15 mg/mL PBNPs), SM1 cells were subjected to PBNP-PTT at varied laser powers (0.75-2.0 W) for ten min in vitro. SM1 cells heated in a laser power-dependent manner, reaching the highest temperature (81.5° C.) at the highest laser power (2.0 W) (FIG. 1B and FIGS. 10A-10E). The time-temperature plots were converted to cumulative equivalent minutes at 43° C. (ΣCEM43), a parameter used to quantify the thermal dose, which illustrated that the thermal doses administered to the SM1 cells were also laser power-dependent (FIG. 1C).

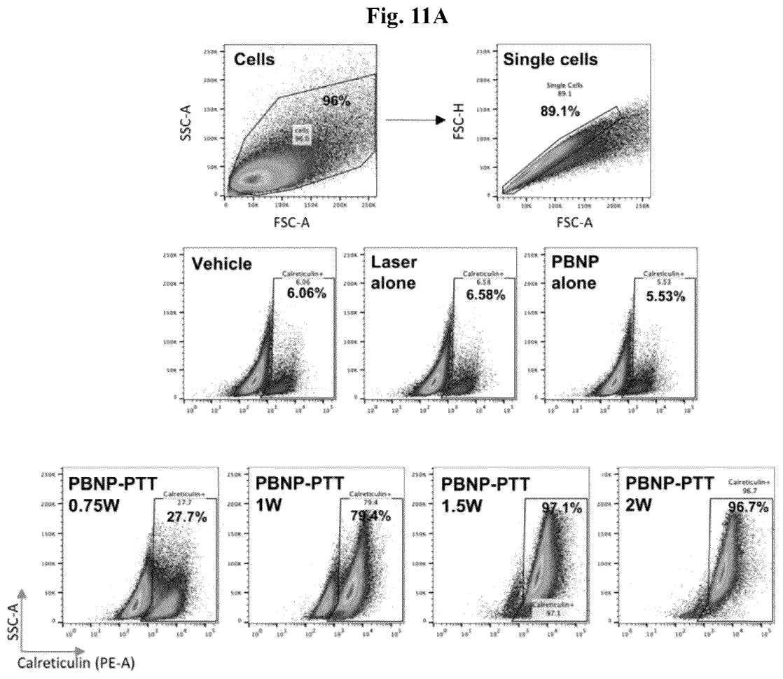

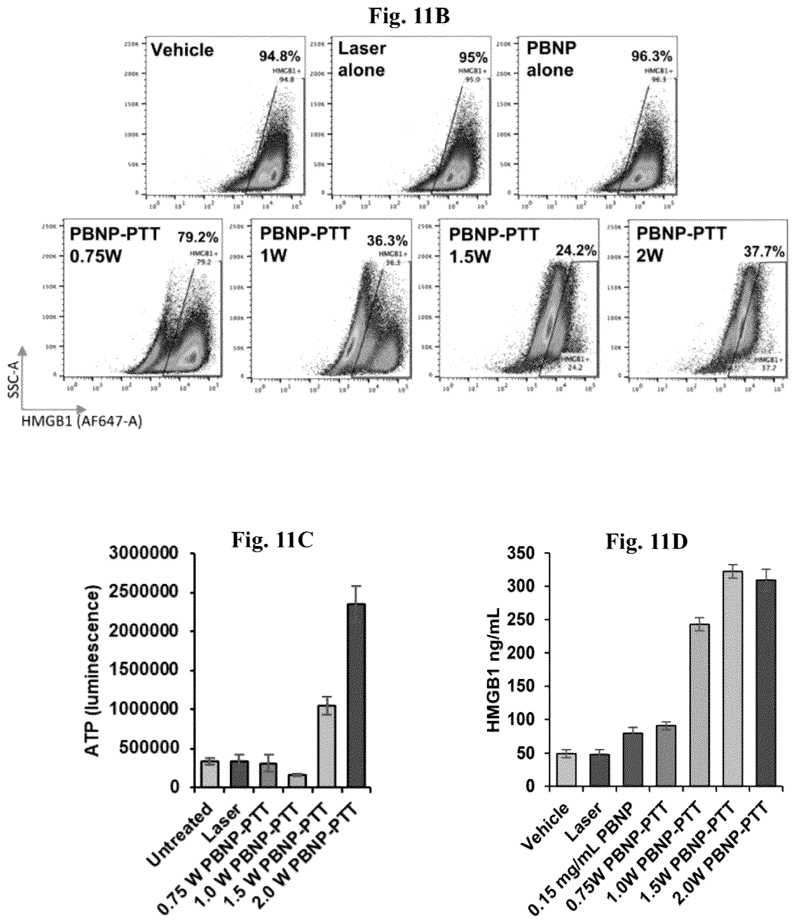

To determine whether the heat generated during PBNP-PTT elicited cytotoxicity and ICD in SM1 cells in vitro, cell death and the consensus biochemical correlates for ICD (i.e., release of intracellular ATP and HMGB1, and upregulation of cell surface calreticulin) were measured. As expected, SM1 cells were killed in a thermal dose-dependent manner, with <10% live cells remaining after treatment with ≥1.5 W laser power (FIG. 1D and FIGS. 10A-10E). PBNP-PTT generated a laser power-and thermal dose-dependent decrease in intracellular ATP of SM1 cells, suggesting it was released (FIG. 1E), with <10% intracellular ATP remaining after treatment with ≥1.5 W laser power. Concurrently, PBNP-PTT generated an increase in SM1 cell surface calreticulin (FIG. 1F) and decreased intracellular HMGB1 (FIG. 1G) as measured by flow cytometry, suggesting the upregulation of cell surface calreticulin and the release of HMGB1 at laser powers ≥1 W. The maximum expression of the ICD correlates was attained at a thermal dose of 8.5-10.5 log(ΣCEM43) (FIGS. 11A-11D).

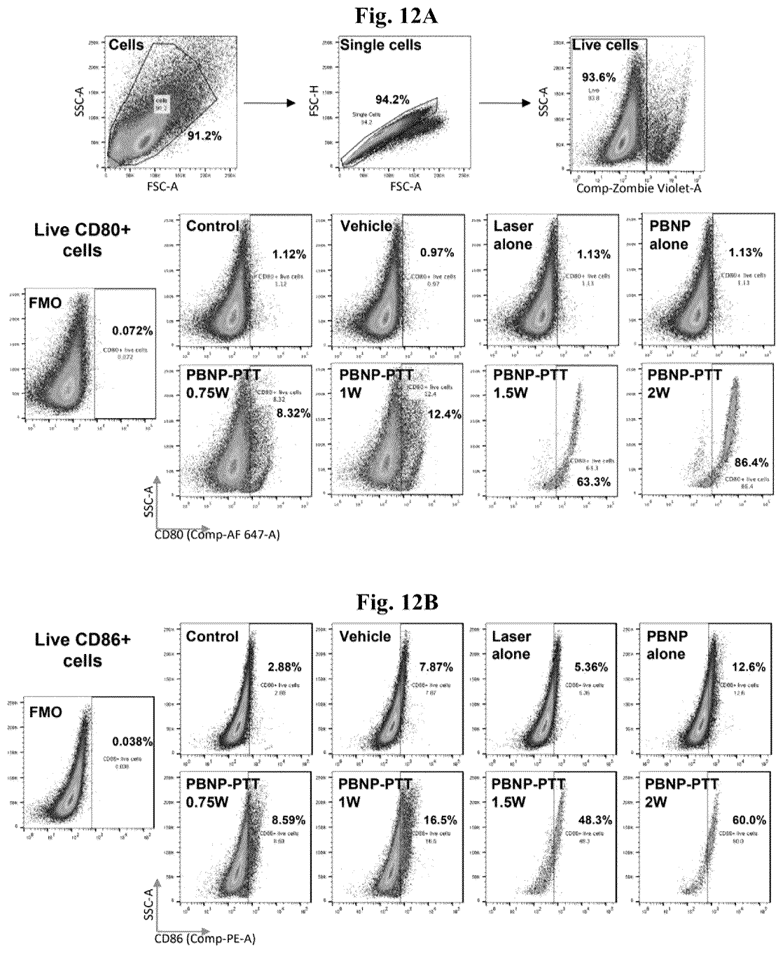

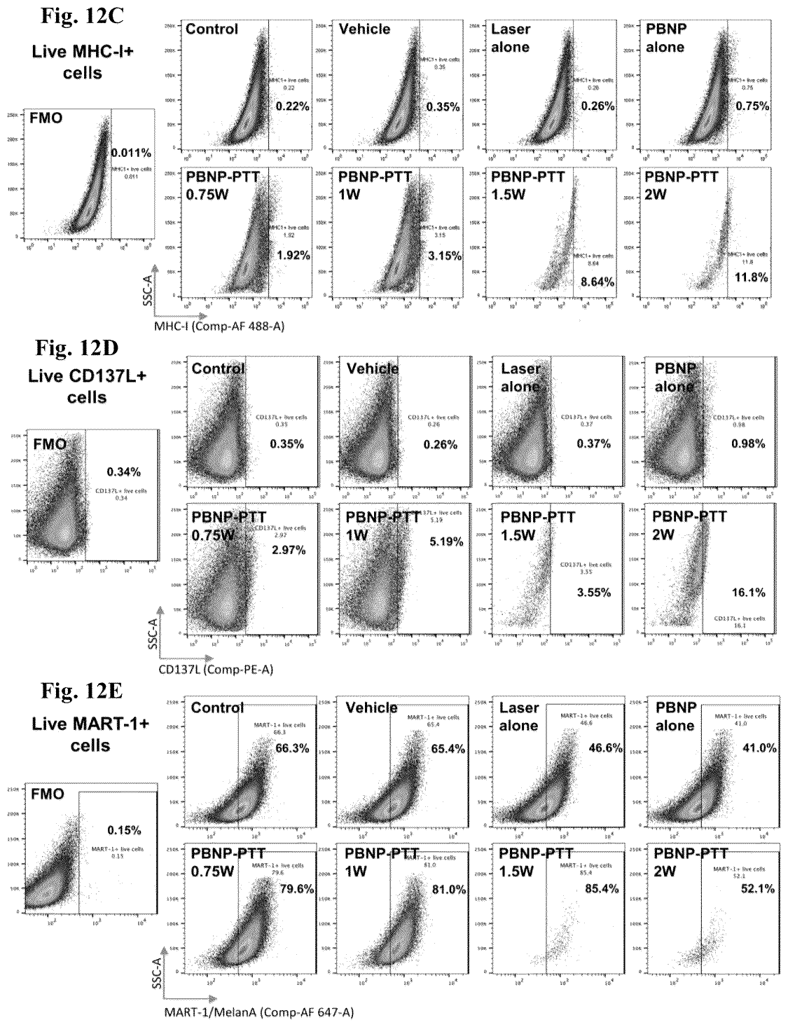

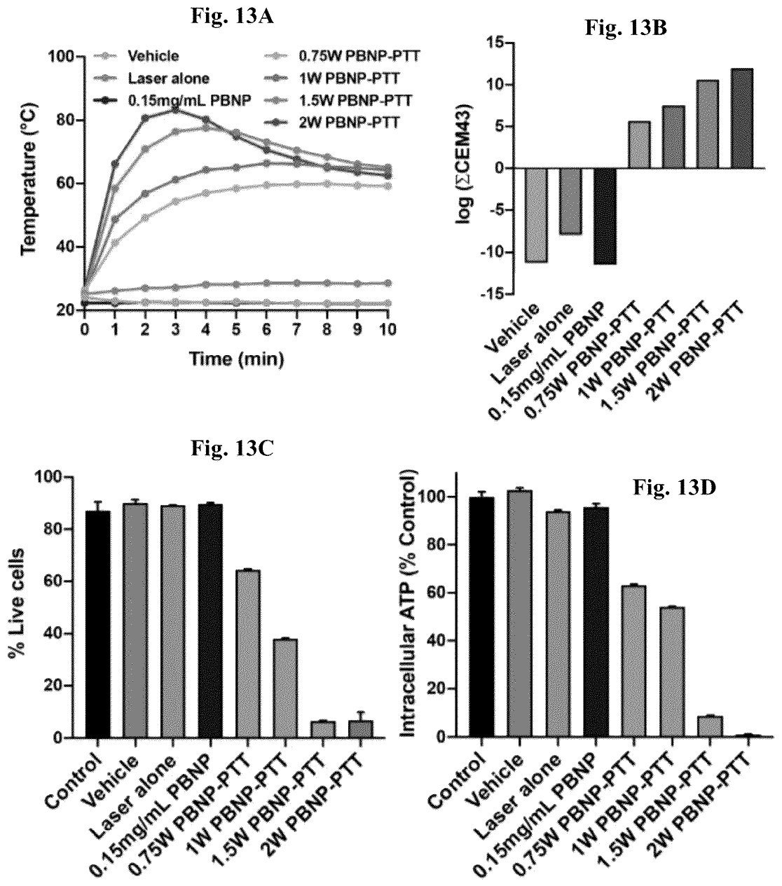

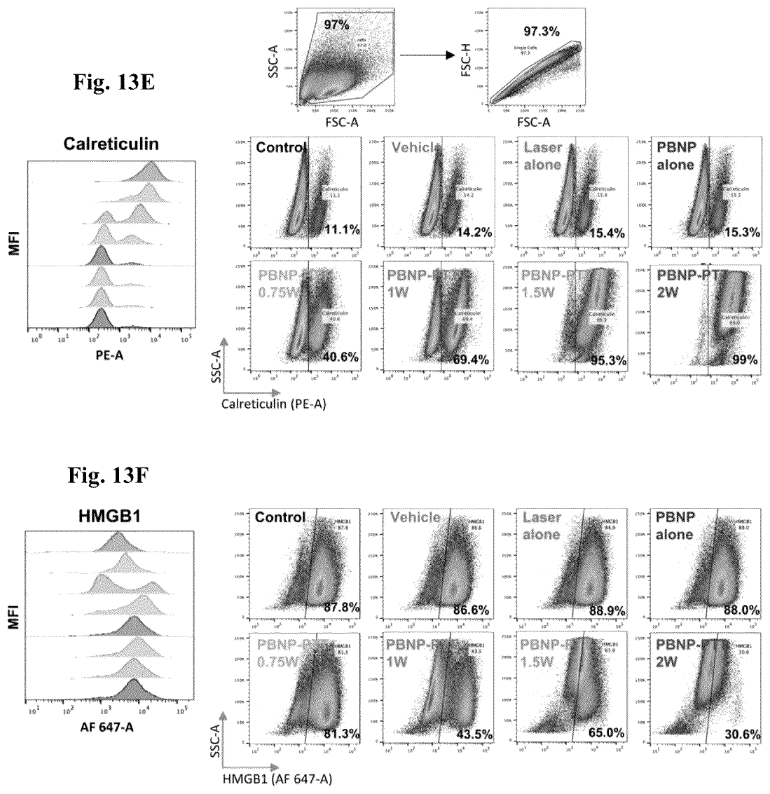

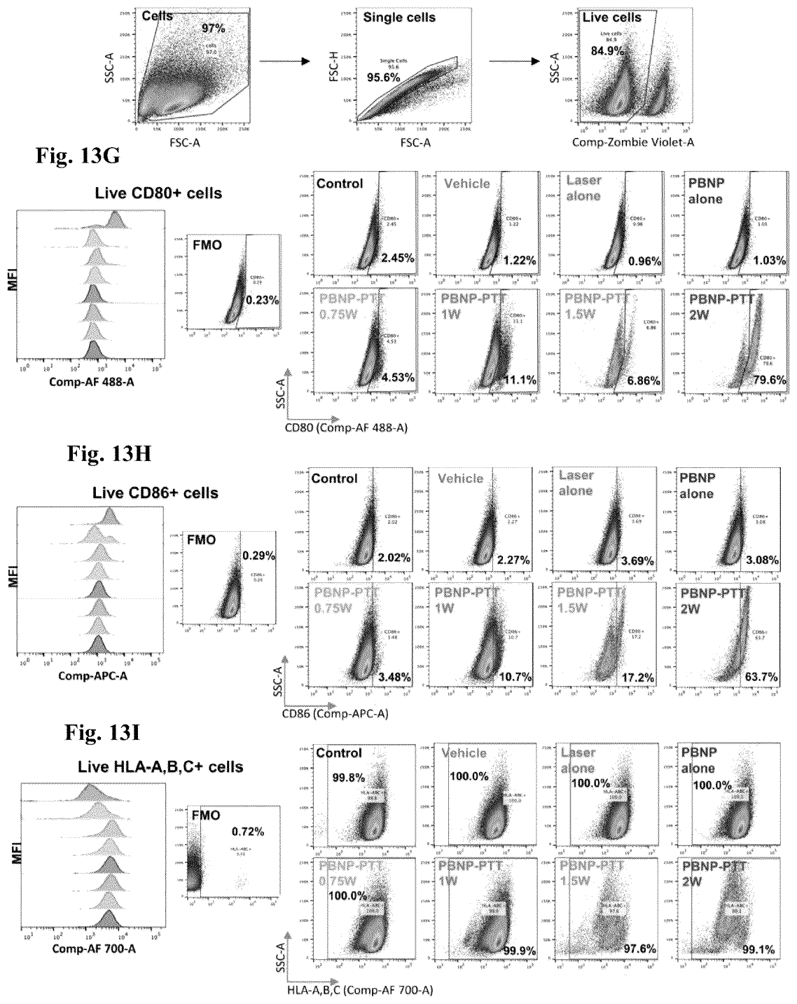

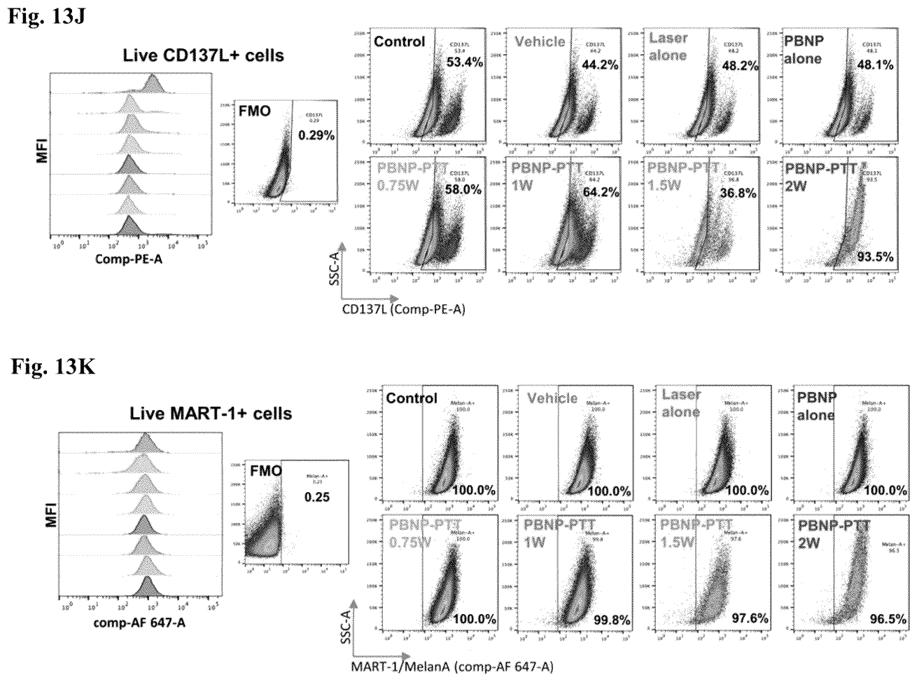

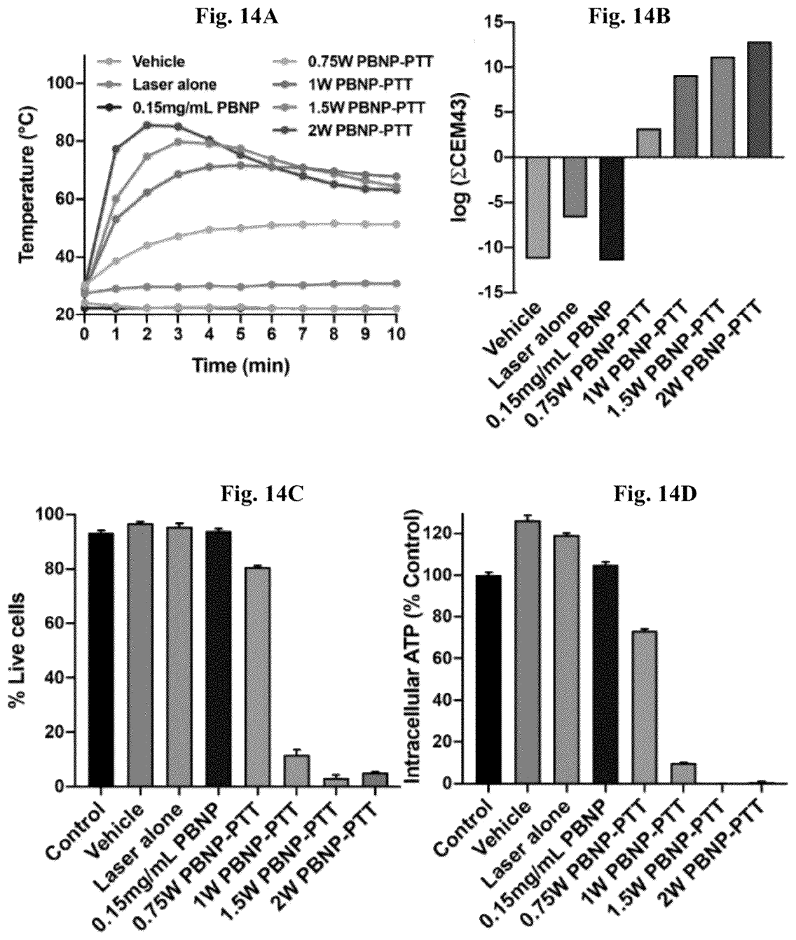

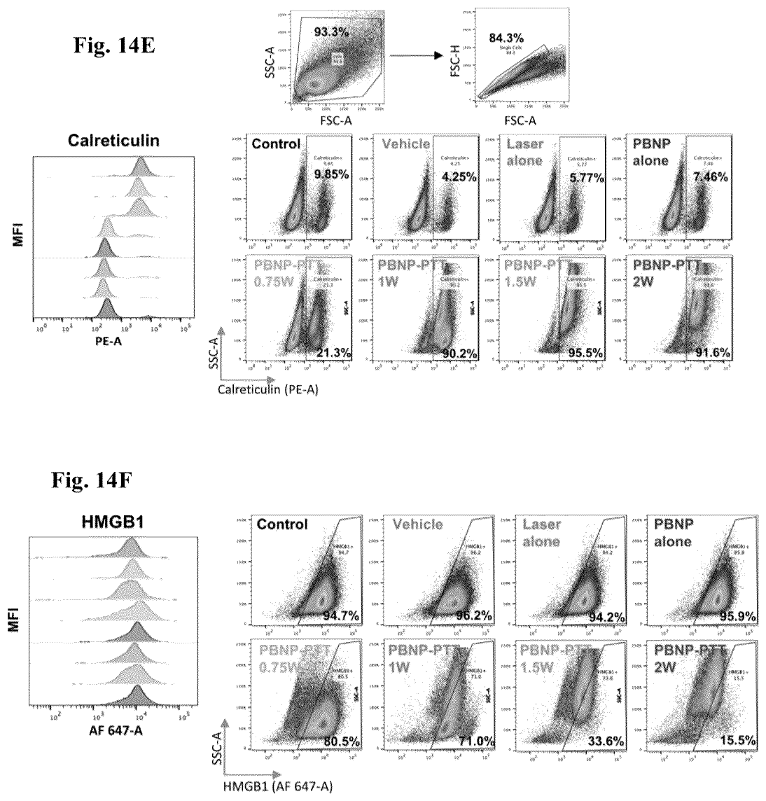

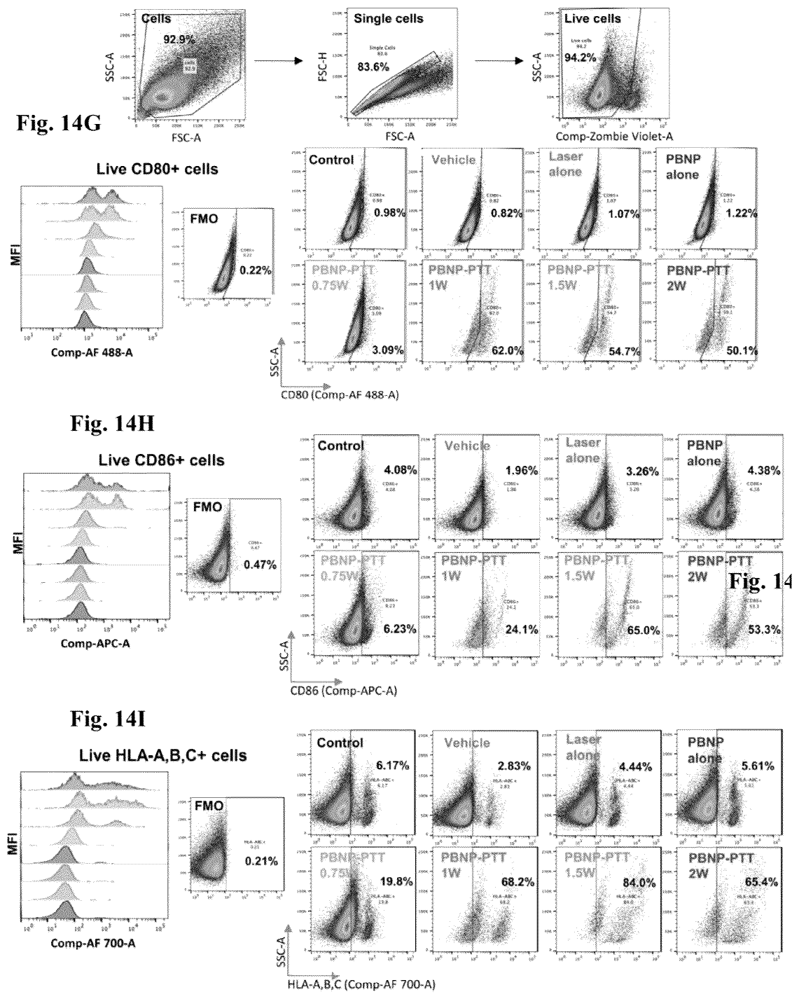

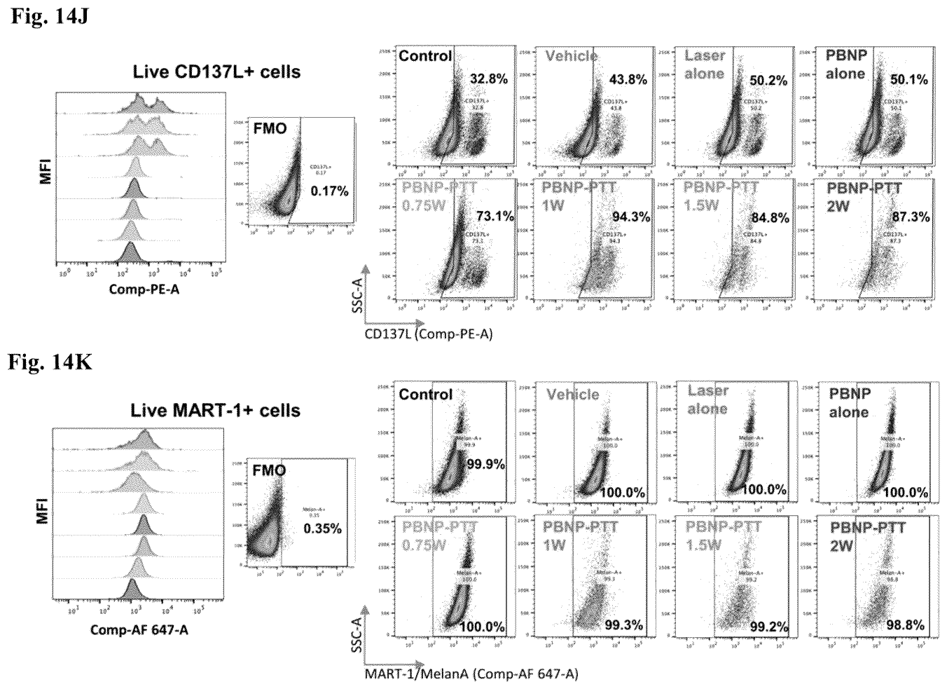

In addition to ICD, the ability of PBNP-PTT to upregulate molecules involved in antigen presentation and immune cell co-stimulation on SM1 cells in vitro was investigated. PBNP-PTT generated laser power-and thermal dose-dependent increases in the expression of CD80 (maximum MFI (mMFI) at 2 W: 4,125; FIG. 1H), CD86 (mMFI: 768; FIG. 1I), MHC-I (mMFI: 2557; FIG. 1J), and CD137 ligand (CD137L) (mMFI: 155; FIG. 1K) on SM1 cells. Melanoma antigen recognized by T cells (MART-1/Melan-A) was also increased with higher PBNP-PTT thermal dose (mMFI: 725 at 1.5 W), suggesting an increase in the antigenicity and immunogenicity potential of the cells in response to treatment (FIG. 1L; see the gating strategy in FIGS. 12A-12E). To establish reproducibility of these effects in human cells, PBNP-PTT was administered to WM9 (FIGS. 13A-13K) and WM793 (FIGS. 14A-14K) human melanoma cell lines harboring the BRAFV600E mutation and predicted to be unresponsive to aPD-1 therapy and illustrated consistent results. Briefly, both WM9 and WM793 tumor cells also generated thermal dose-dependent increases in ICD biochemical correlates and molecules involved in antigen presentation and immune cell co-stimulation. Maximum cell death and immunogenicity, as measured by the aforementioned markers, was elicited with PBNP-PTT using a 2 W laser power (12.8 log(ΣCEM43)) for WM9 cells and 1.5 W laser power (10.6 log(ΣCEM43)) for WM793 cells correlating with the effects observed in murine SM1 cells.

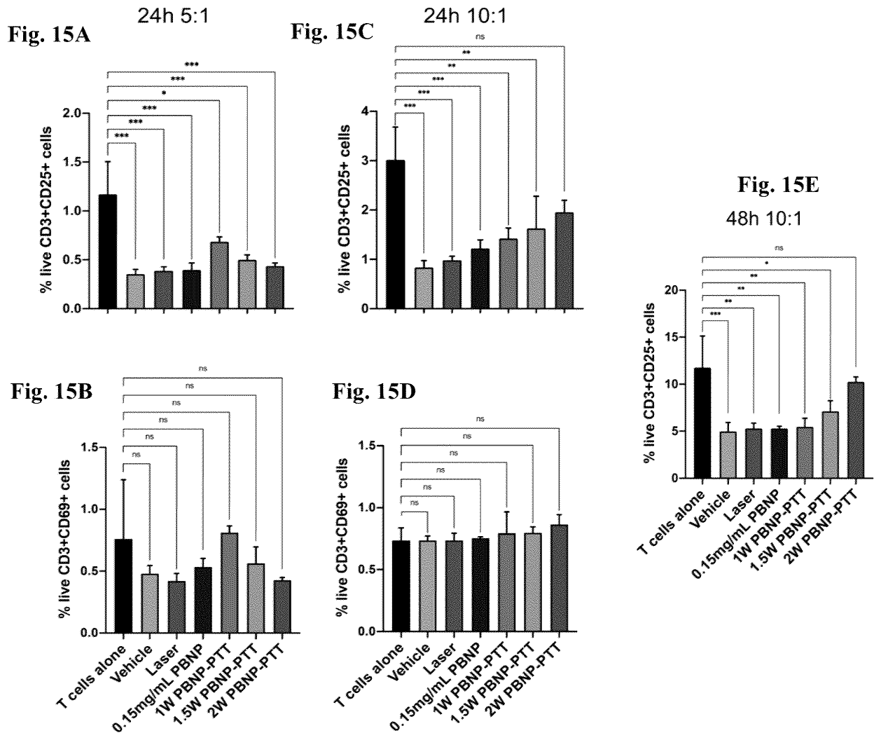

Because PBNP-PTT upregulated molecules involved in antigen presentation and immune cell co-stimulation, experiments to uncover whether SM1 cells treated with PBNP-PTT could directly activate T cells were performed. For this purpose, T cells were isolated from the spleens of naïve mice (not exposed to SM1 cells) and co-cultured ex vivo at a 5:1 effector-to-target cell (E:T) ratio with SM1 cells treated in vitro with either vehicle (water), PBNPs (0.15 mg/mL), or PBNP-PTT (0.15 mg/mL PBNP +10 minute laser illumination at 1 W, 1.5 W, or 2 W). After 48 hours, expression of the activation marker CD69 was significantly increased on T cells after co-culture with SM1 cells treated with PBNP-PTT at all thermal doses tested (FIG. 1M). The highest T cell activation was observed with 1.5 W PBNP-PTT (14.7% CD3+/CD69+). Expression of CD69 and CD25, another T cell activation marker, was also measured in response to PBNP-PTT-treated SM1 cells at 5:1 and 10:1 E: T ratios after 24 and 48 hours of co-culture; no other tested condition elicited direct T cell activation as measured by increased CD69 or CD25 expression (FIGS. 15A-15E). Co-culture with SM1 cells (after vehicle and PBNP-PTT treatments) generated small but statistically significant decreases in CD25 expression (FIGS. 15A-15E). These data suggest that activation of T cells by surviving PBNP-PTT-treated cells, in a similar manner to antigen-presenting cells (APCs), can be achieved under specific thermal dose conditions (FIG. 1N).

The overall goal of these described in vitro studies was to establish the bounds of PBNP-PTT thermal dose that elicits immunogenicity in melanoma cells. Based on these studies, it was observed that the optimal PBNP-PTT was generated in vitro using a laser power of 1.5-2 W, which corresponds to 10.3-10.5 log(ΣCEM43) for the tested melanoma cells. These thermal doses enabled PBNP-PTT to generate optimal effects, that is, high percentage of tumor cell killing, induction of ICD, upregulation of immunostimulatory molecules, and direct T cell activation. Thus, PBNP-PTT should be administered at a thermal dose above ˜10.3 log (ΣCEM43) for optimal cell death and immunogenicity.

### Example 3

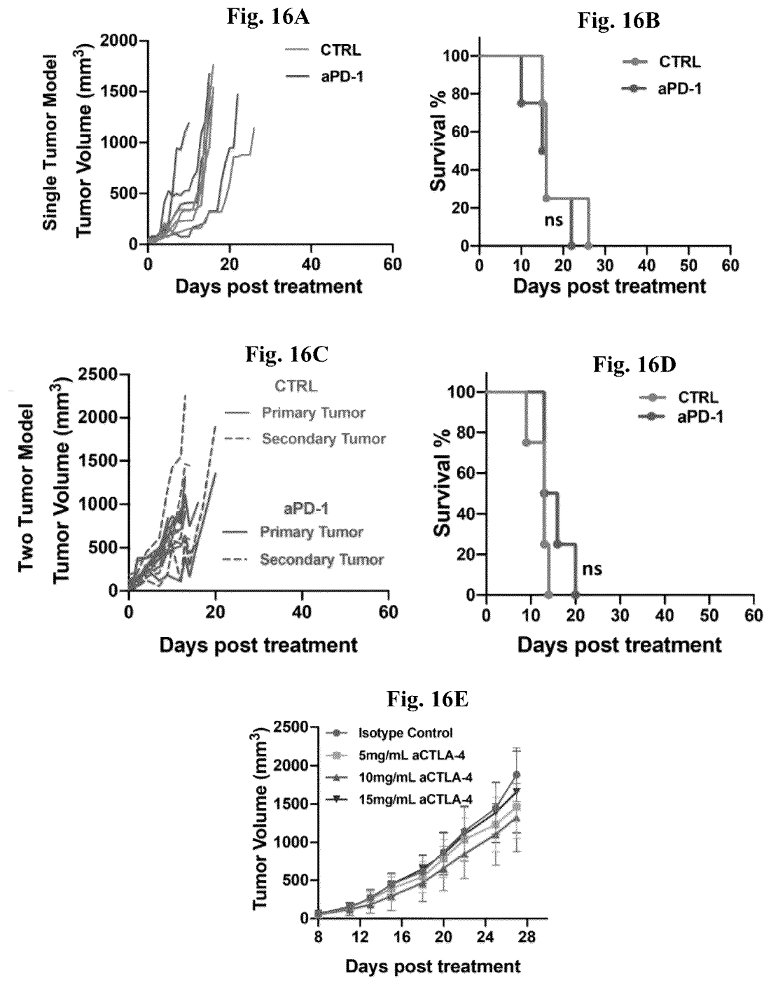

In another exemplary method, PBNP-PTT eliminated local SM1 tumors but could not eliminate a distant tumor and was not improved with the addition of a PD-1 ICI therapy. To determine the efficacy of ICI therapy on SM1 melanoma, the effects of aPD-1 and aCTLA-4 mAb immunotherapy were separately investigated in the SM1 melanoma mouse model. As predicted based on its mutation status (BRAFV600E), it was verified that, using the specific dosing scheme disclosed herein, neither aPD-1 nor aCTLA-4 ICI therapy was able to significantly slow the growth of SM1 tumors nor improve the survival of SM1 tumor-bearing mice (FIGS. 16A-16E).

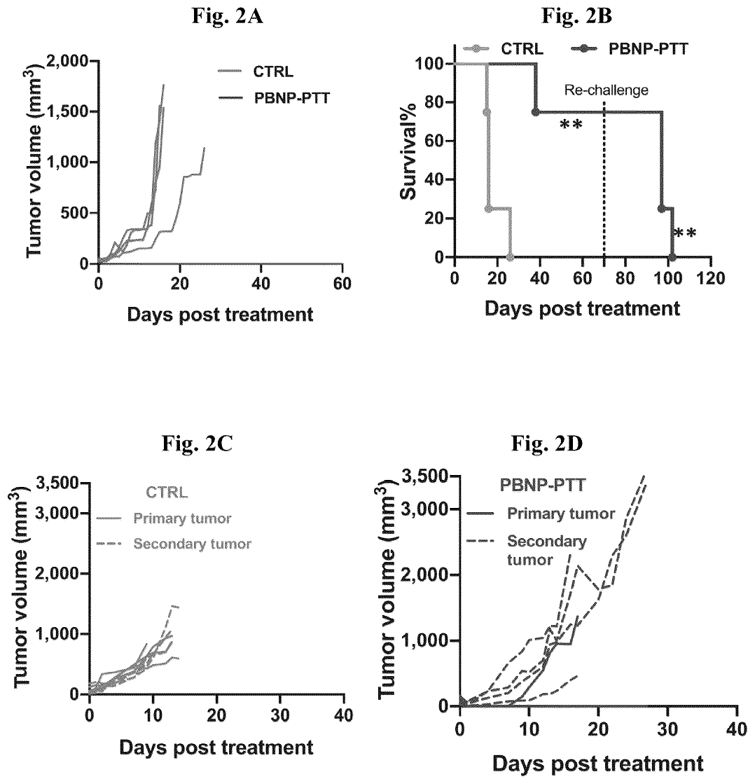

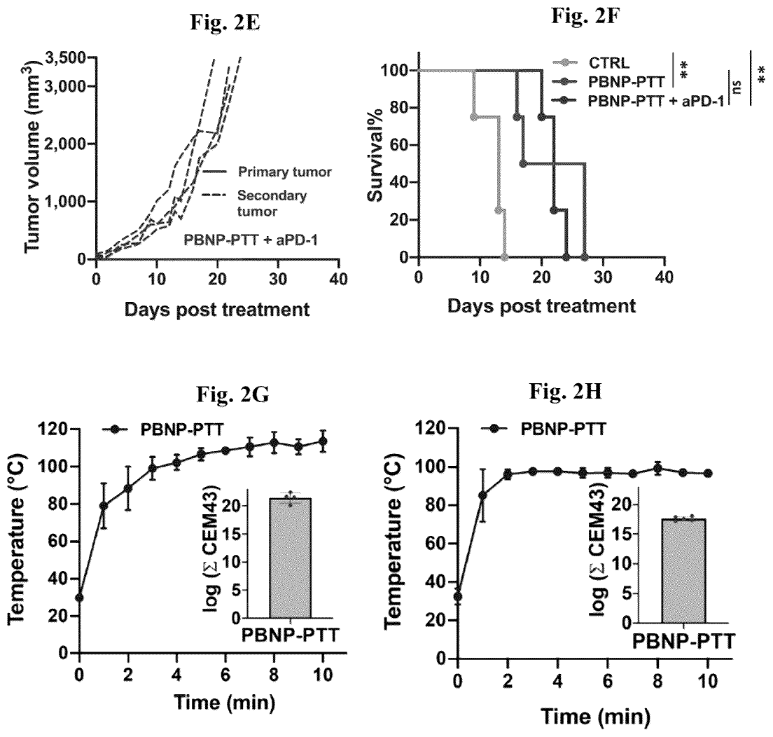

Having established that SM1 melanoma did not respond to ICI therapy at the doses tested, and that PBNP-PTT enabled SM1 cell death and immunogenicity in vitro, it was next sought to understand the effect of PBNP-PTT on SM1 melanoma in vivo. SM1 tumor-bearing mice were treated with PBNP-PTT or left untreated. PBNP-PTT eliminated 100% of treated tumors, while tumors of mice left untreated (CTRL) progressed (FIG. 2A and 2G), resulting in a significant improvement in survival for mice treated with PBNP-PTT (median survival (MS): not achieved in the study) versus CTRL (MS: 16 days) (FIG. 2B). Long-term surviving PBNP-PTT-treated mice (n=3, 75%) were rechallenged with SM1 tumors ˜70 days after initial treatment. Zero percent (%) of the rechallenged mice rejected the tumor, and 100% succumbed to tumor burden within 32 days after rechallenge (FIG. 2B). These data suggested that although PBNP-PTT was effective for local tumor control in vivo, it did not generate an effective immunological memory response.

To determine whether local treatment with PBNP-PTT generated a systemic immune response able to impact the growth of a secondary tumor, SM1 cells were simultaneously inoculated into contralateral flanks of mice to generate two synchronous tumors. PBNP-PTT was administered to one tumor (designated “primary” tumor), and the other tumor was left untreated (designated “secondary” tumor). While PBNP-PTT-treated primary tumors were eliminated, the secondary untreated tumor growth was unchanged compared to CTRL (FIGS. 2C-2D and 2H). The tumor burden endpoints for each individual tumor in the CTRL group were lower than in the groups receiving PBNP-PTT because mice in the CTRL group had two tumors (thus lowering the endpoint criteria) whereas those in the PBNP-PTT-treated groups had only one tumor. These results suggested that although PBNP-PTT was effective in treating single tumors, the immunogenic effects could not eliminate established distal disease, and thus may benefit from combination with immunotherapy. However, mice treated with the combination treatment of PBNP-PTT+aPD-1 did not exhibit any benefit over PBNP-PTT alone in this two-tumor model (MS: 22) (FIGS. 2E-2F). Although survival was marginally improved for mice treated with PBNP-PTT (MS: 22 days) over CTRL (MS: 13.5 days), all PBNP-PTT and PBNP-PTT+aPD-1-treated mice succumbed to tumor burden and there were no long-term survivors (FIG. 2F). These data suggested that PBNP-PTT may require a different complementary immunotherapy intervention to exert improved therapeutic benefit for this melanoma model.

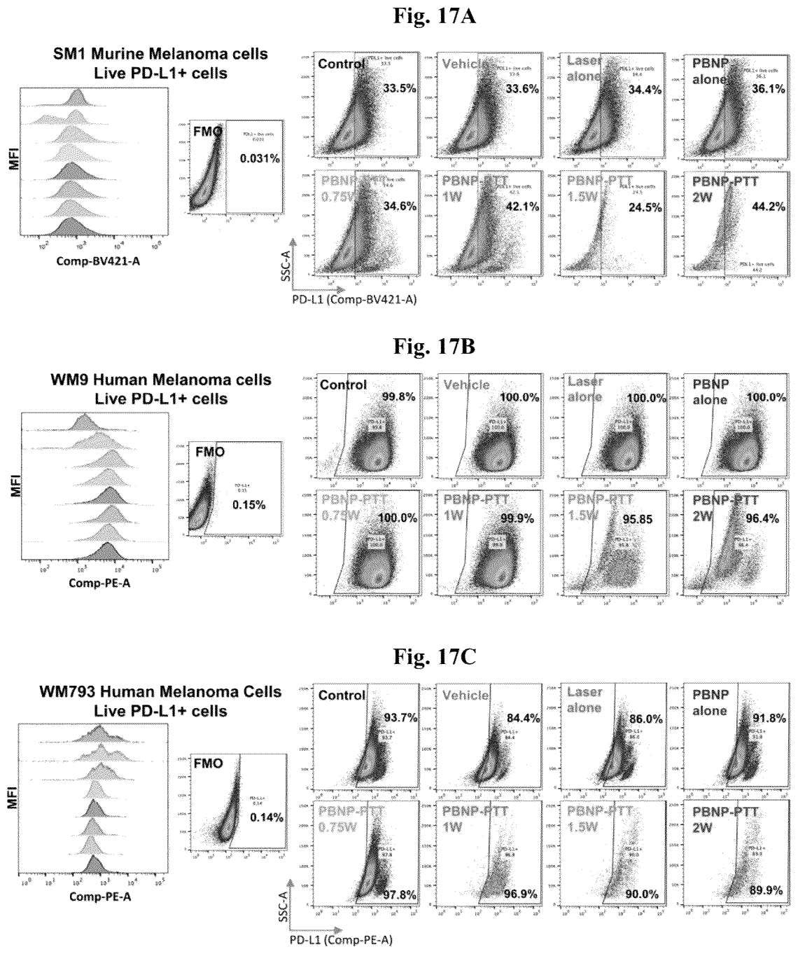

To uncover whether the lack of responsiveness to PBNP-PTT+aPD-1 therapy was due to a lack of expression of PD-L1, a known ligand for PD-1, SM1 cells were treated with varying doses of PBNP-PTT. PD-L1 was unchanged or decreased from basal levels in response to all tested conditions of PBNP-PTT in SM1 melanoma cells and in both human melanoma cell lines (FIGS. 17A-17C), suggesting that the effects of PBNP-PTT were not improved by combining with aPD-1 therapy.

### Example 4

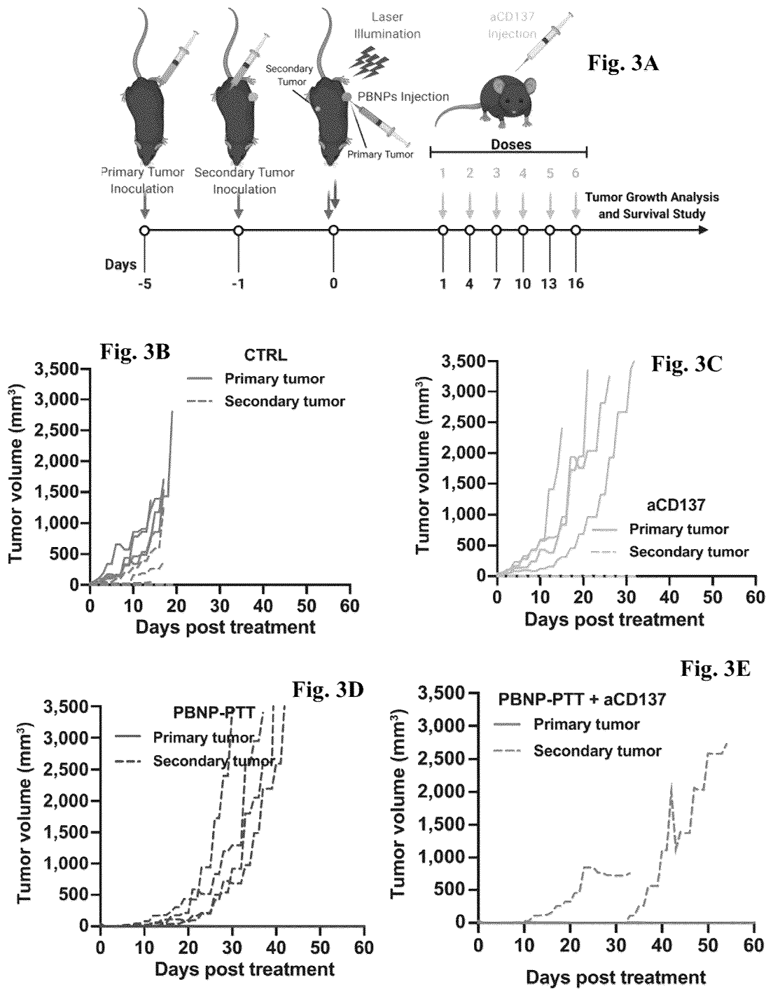

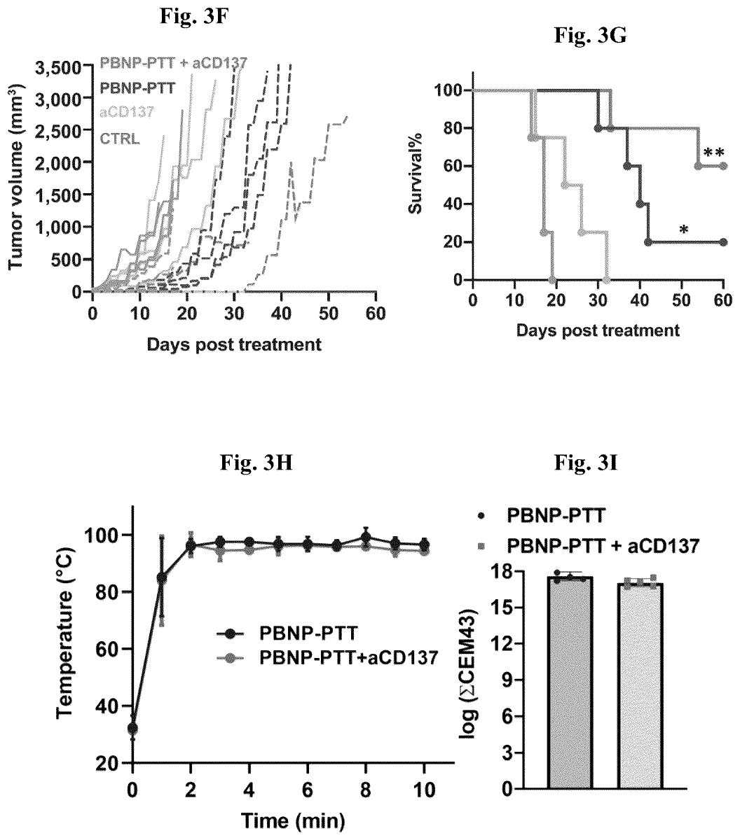

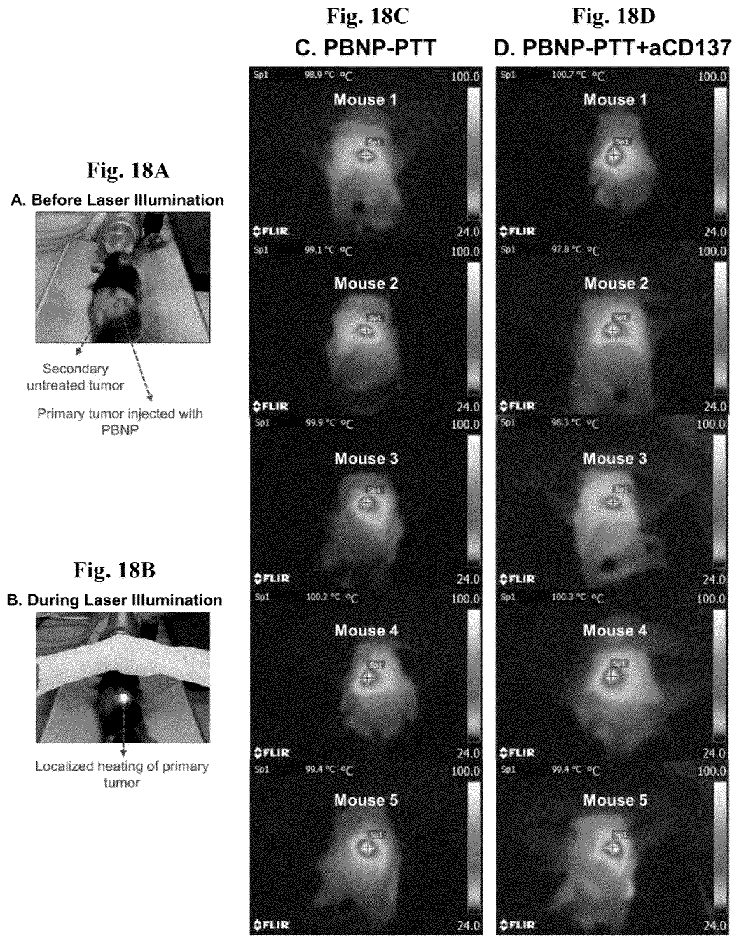

In another exemplary method, PBNP-PTT combined with aCD137 generated an abscopal effect that significantly improved the long-term survival of metachronous tumor-bearing mice. In light of the examples above illustrating the increase of CD137L on SM1 melanoma cells in response to PBNP-PTT in vitro, aCD137, a T cell agonistic mAb, was utilized as a second component of a nanoimmunotherapy in combination with PBNP-PTT. It was next sought to uncover if the antitumor effects of the combination treatment could be effective in a model of distal melanoma. To test this, bilateral metachronous SM1 tumors were utilized with four days between tumor cell inoculations. The treatment groups were: CTRL (untreated), PBNP-PTT, aCD137, and PBNP-PTT+aCD137. In CTRL animals, both tumors were untreated. When used, PBNP-PTT was administered once locally to only the first established (“primary”) tumor, while the secondary tumor was left untreated (FIGS. 18A-18D). When used, aCD137 was systemically delivered via intraperitoneal (i.p.) administration. This setup enabled observation of a potential abscopal effect (FIG. 3A). Primary and secondary tumors of CTRL mice rapidly progressed (FIGS. 3B and 3H-3I). Although aCD137 treatment did not affect primary tumor growth compared to CTRL, it prevented 100% of secondary tumors in the metachronous two-tumor model (FIG. 3C), suggesting that it could mount an effective antitumor immune response to attack a nascent tumor, but did not have the cytotoxic capability to eliminate an established tumor. 100% of PBNP-PTT-treated primary tumors were eliminated, and secondary tumors exhibited significantly delayed growth kinetics compared to CTRL (FIG. 3D). This pattern suggested that PBNP-PTT exerted both a cytotoxic effect to the primary tumor and a suboptimal abscopal antitumor immune response that was able to slow untreated secondary tumor growth, although unable to completely prevent its eventual growth. Strikingly, 60% of mice treated with PBNP-PTT+aCD137 exhibited complete primary tumor elimination and prevention of secondary tumor growth (FIG. 3E), suggesting the synergistic advantage of combining the two treatment modalities. Comparative tumor growth rates were illustrated in superimposed curves (FIG. 3F). These effects on tumor growth resulted in significantly improved survival benefit for mice treated with PBNP-PTT (MS: 40 days) or PBNP-PTT+aCD137 (MS: not achieved during the study) compared to no treatment (MS: 17 days) and aCD137 treatment (MS: 17 days) (FIG. 3G). These data suggested that combining local PBNP-PTT with systemic aCD137 administration enabled robust abscopal efficacy against SM1 tumors in vivo.

### Example 5

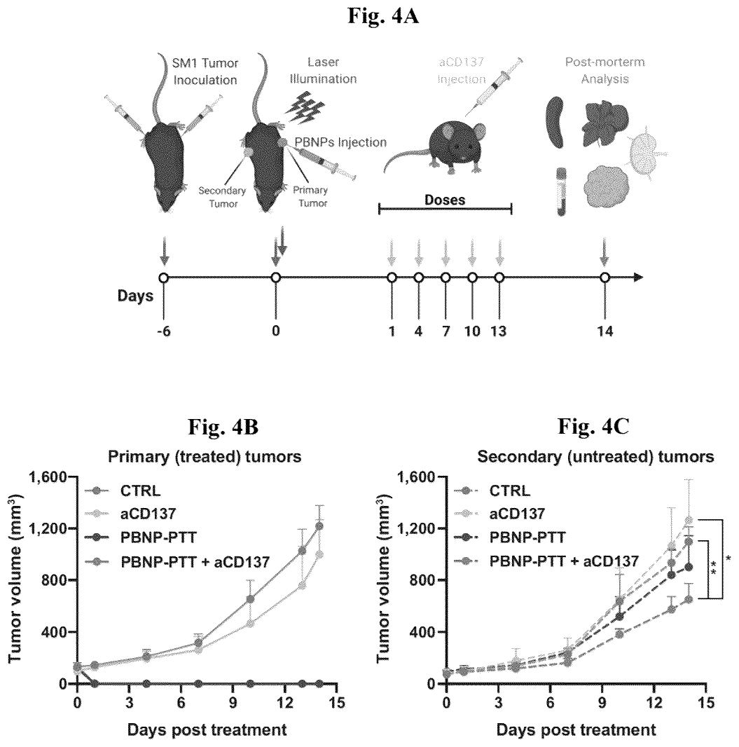

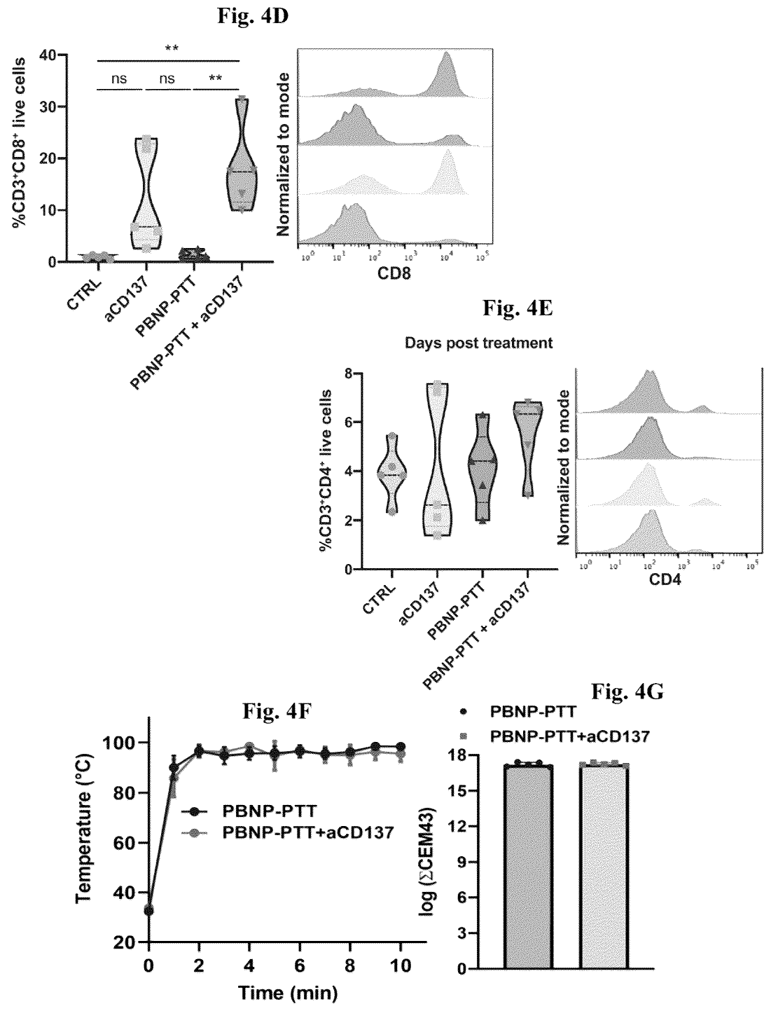

In yet another exemplary method, PBNP-PTT combined with aCD137 generated an abscopal antitumor effect via tumor-infiltrating CD8+ T cells and systemic activation of DCs and T cells. Having illustrated the treatment benefit of PBNP-PTT+aCD137 on bilateral SM1 melanoma tumors in vivo in the examples herein, it was next sought to identify the key mechanistic components of immunity driving the observed abscopal efficacy. In order to analyze infiltrating immune cells in an established secondary tumor, a synchronous, rather than metachronous, bilateral tumor model was generated. This model enabled the collection of secondary tumors not present in all treatment groups in the metachronous model. The treatment groups were: CTRL (untreated); PBNP-PTT; aCD137; and PBNP-PTT+aCD137. When used, PBNP-PTT was administered once locally to only one tumor denoted as “primary,” while “secondary” tumors were left untreated. When used, aCD137 was systemically delivered via i.p. administration (FIG. 4A). CTRL mice rapidly grew both primary and secondary tumors (FIGS. 4B-4C and 4F-4G). Unlike the metachronous model, aCD137 was unable to impact the growth of either tumors and all aCD137-treated mice rapidly grew both primary and secondary tumors. PBNP-PTT, alone and in combination with aCD137, eliminated 100% of primary tumors (FIGS. 4B-4C). PBNP-PTT+aCD137 significantly slowed secondary tumor growth (1.68-fold reduction in tumor volume compared to CTRL at the day of euthanasia), confirming the abscopal effect (FIG. 4C). Secondary tumors were not eliminated, as they were in mice treated with PBNP-PTT+aCD137 in the metachronous model, since the synchronous model comprised two established tumors, thus representing a higher initial tumor burden.

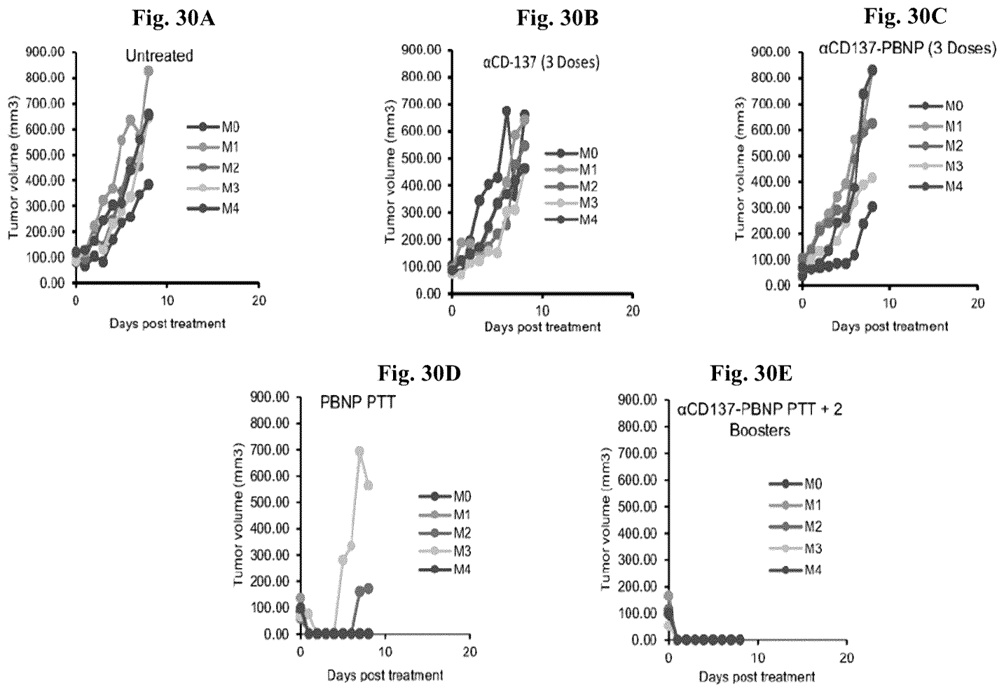

Additionally, tumor-bearing mice were randomly divided into five treatment groups: 1) vehicle (PBS); 2) aCD137; 3) aCD137-PBNP (no laser irradiation); 4) PBNP-PTT; and 5) aCD137-PBNP-PTT followed by 2 boosters (aCD137-PBNP with no laser irradiation) (FIGS. 30A-30E). The aCD137-PBNP-PTT treatment followed by 2 booster treatments greatly decreased tumor volume.

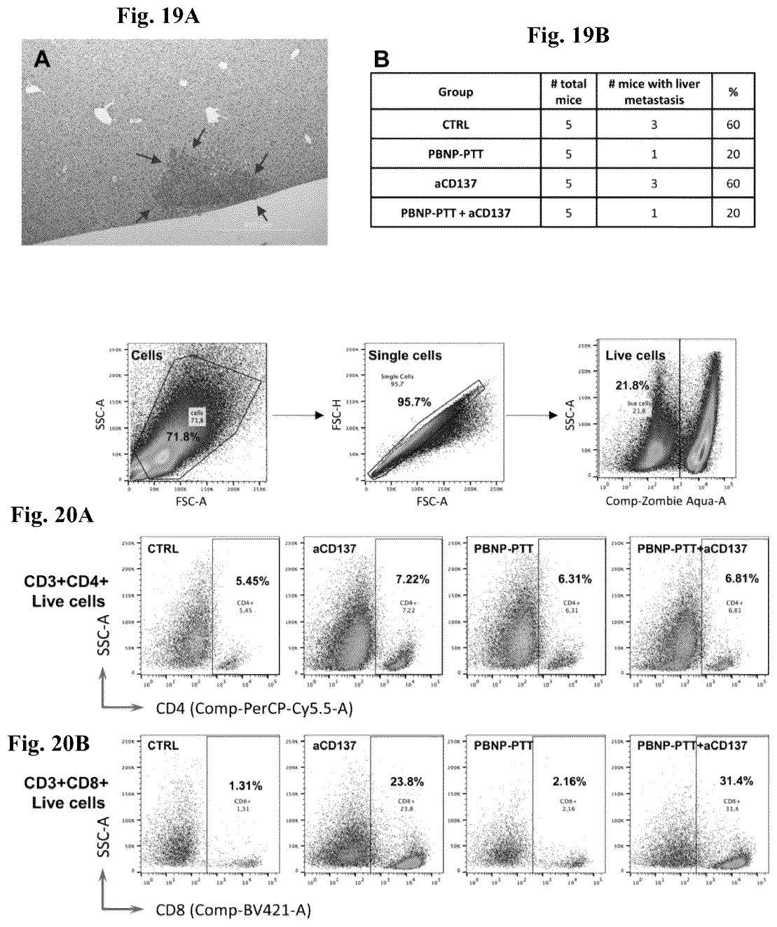

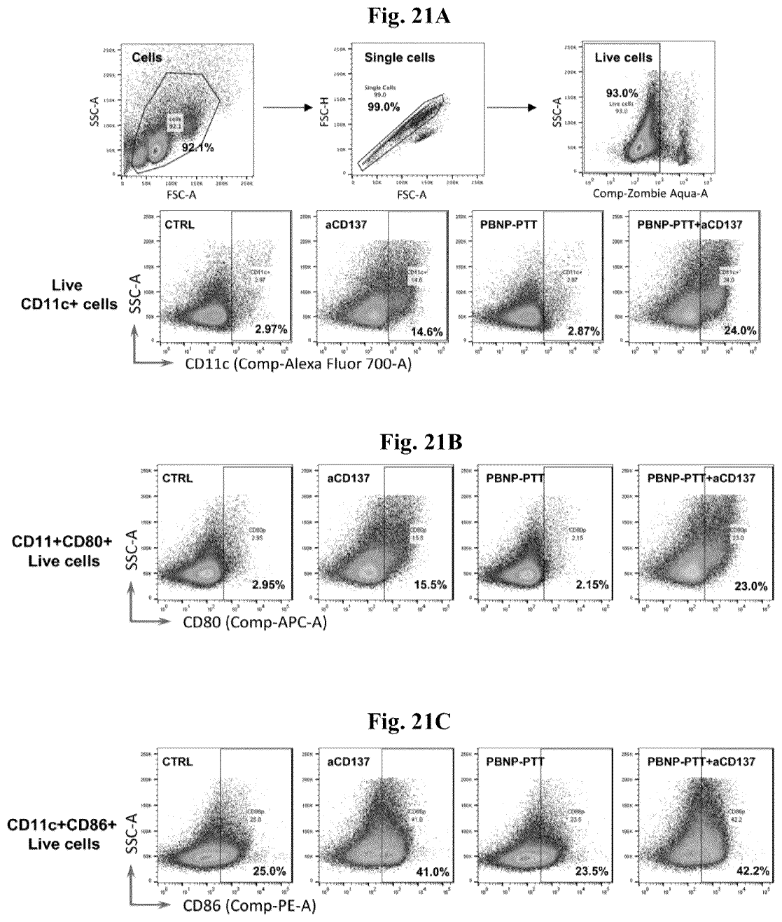

After 14 days (a timepoint established before treatment), mice were euthanized, and tumors, lymph nodes, spleens, blood, and livers were harvested for analysis. To illustrate the abscopal effect on metastasis, livers were harvested and analyzed for the presence of metastatic foci. Sixty percent (60%) of livers from CTRL and aCD137-treated mice harbored tumor foci (>10 tumor cells) (FIGS. 19A-19B), as compared to mice treated with PBNP-PTT (20%) or PBNP-PTT+aCD137 (20%), suggesting that PBNP-PTT prevented the occurrence of hepatic metastases via abscopal immune effects.

To understand the mechanism through which PBNP-PTT+aCD137 exerted robust abscopal effect, the presence of tumor-infiltrating lymphocytes (TILs) in secondary tumors was investigated by flow cytometry. The percentage of CD8+ T cells observed in tumors was significantly higher in PBNP-PTT+aCD137-treated mice (17%; FIG. 4D and FIGS. 20A-20B) compared with CTRL (<1%) and PBNP-PTT-treated mice (<1%), suggesting that CD8+ T cell infiltration may drive the responses seen in the secondary tumors. Although not statistically significant, aCD137 monotherapy also marginally increased CD8+T cell infiltration into secondary tumors (FIG. 4D and FIGS. 20A-20B). The content of CD4+ T cells in tumors was statistically identical across treatment groups (2%-7%; FIG. 4E and FIGS. 20A-20B).

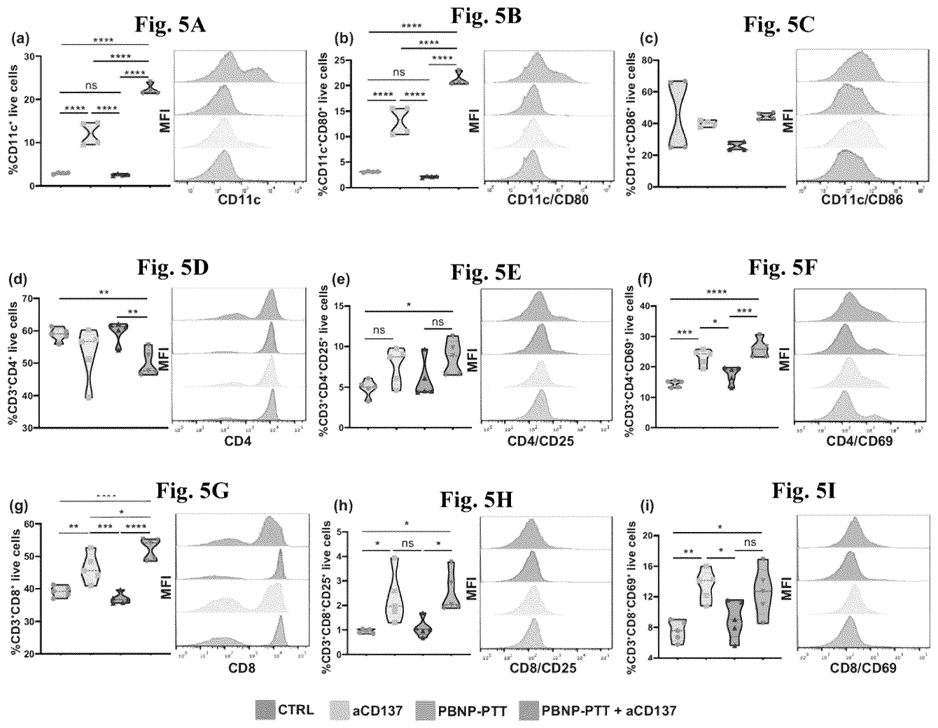

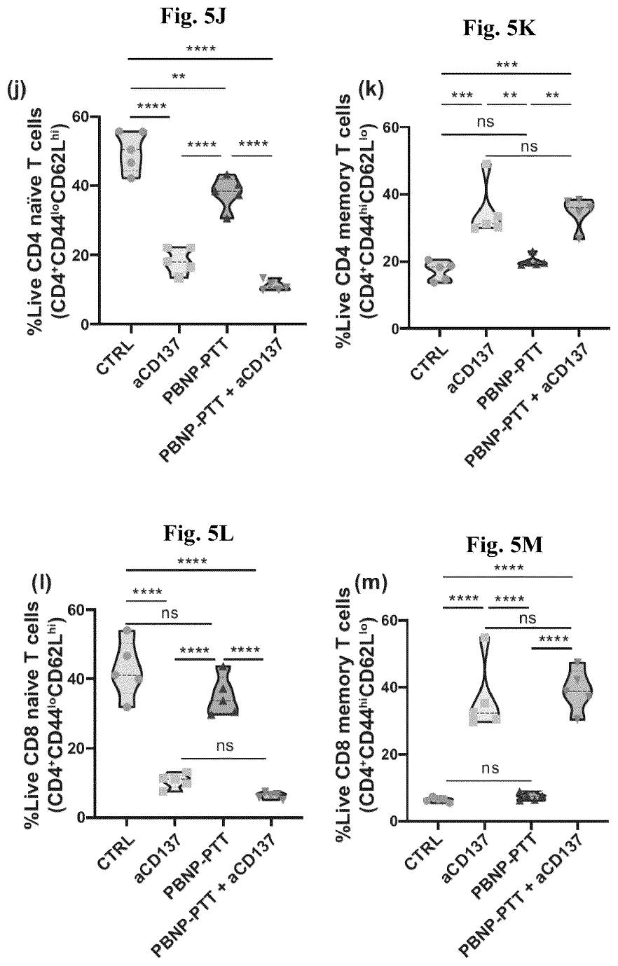

PBNP-PTT+aCD137-treated mice also exhibited significantly higher DC maturation and activation in the inguinal lymph nodes as measured by a significantly increased proportion of CD11c+cells (21.6%, FIG. 5A and FIGS. S21A-21C) and CD11c+/CD80+ cells (20.4%, FIG. 5B and FIGS. S21A-21C) compared to all other groups. CD11c+/CD86+ cells were not statistically different across treatment groups compared to CTRL, likely due to the high variance of CD11c+/CD86+ DCs in the lymph nodes of CTRL mice (24%-66%) (FIG. 5C). Treatment with aCD137 also generated significantly increased CD11c+ (12.2%) and CD11c+/CD80+ (13.3%) cells in the lymph nodes compared to CTRL and PBNP-PTT treatments, but these populations were significantly lower than those found in mice treated with PBNP-PTT+aCD137. These observations suggested that PBNP-PTT+aCD137 elicited systemic antitumor immune responses driven by DCs. Because these analyses were performed 14 days after the administration of PBNP-PTT, the acute effects of PBNP-PTT on DC activation and maturation were not measured. However, it was predicted that PBNP-PTT would generate short-term increases in CD80+ and CD86+ DC populations in the lymph nodes.

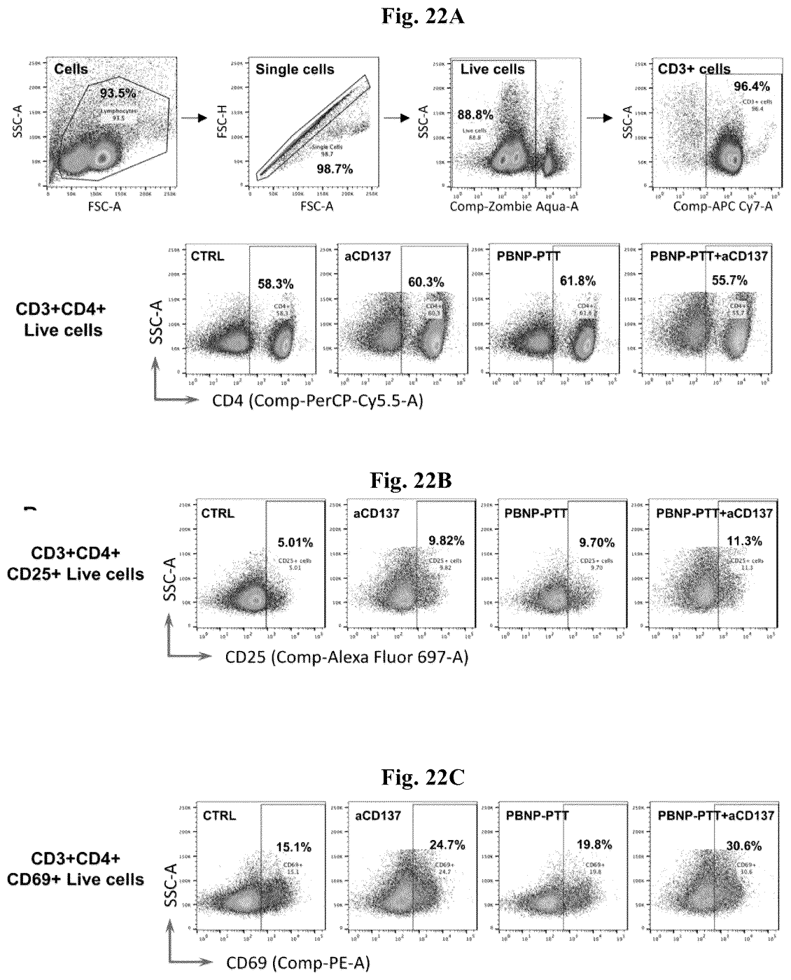

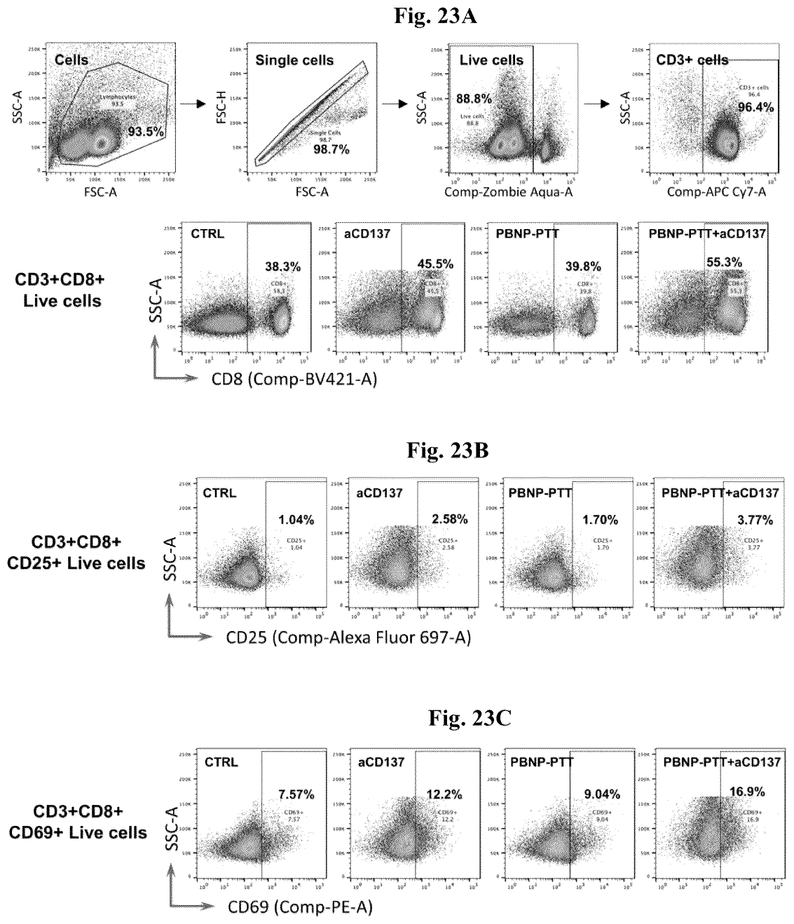

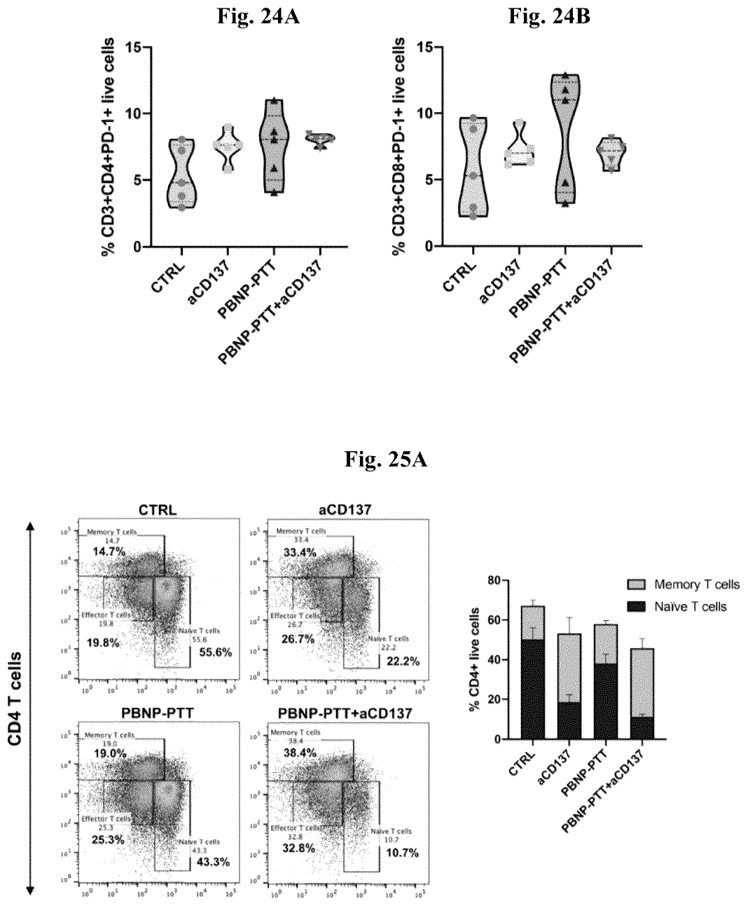

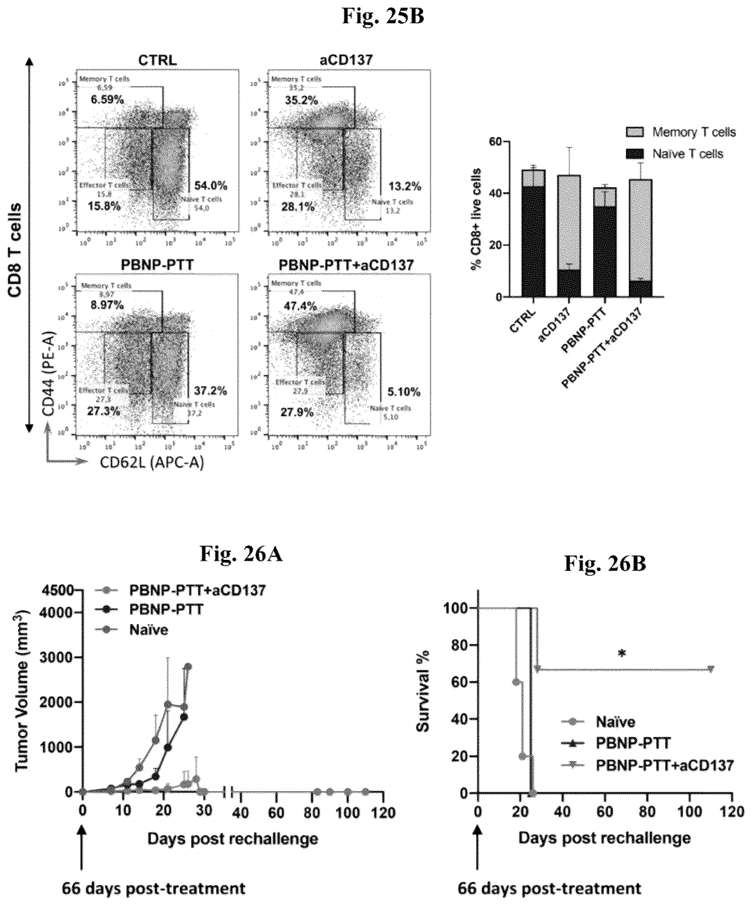

To complement the observations in the secondary tumors and lymph nodes, spleens were analyzed for T cell activation by flow cytometry 14 days after treatment initiation (24 hours after final aCD137 administration). PBNP-PTT+aCD137 treatment generated a significant decrease in CD4+splenic T cells (47%) compared to CTRL (59%) and PBNP-PTT (61%) treatment (FIG. 5D and FIGS. 22A-22C). Interestingly, despite the decrease in CD4+ T cell populations in PBNP-PTT+aCD137-treated mice, the CD4+ T cells appeared to be significantly activated, as seen by their increased expression of CD25 (9% vs. 5% in CTRL; FIG. 5E and FIGS. 22A-22C) and CD69 (25.7% vs. 14% in CTRL; FIG. 5F and FIGS. 22A-22C). The aCD137-treated mice also exhibited significantly increased CD4+/CD69+ cells (24.2%) compared to CTRL and PBNP-PTT treatments. PBNP-PTT+aCD137 treatment generated a significant increase in splenic CD8+ T cells (64%) compared to CTRL (40%), aCD137 (45%), and PBNP-PTT (37%) (FIG. 5G and FIGS. 23A-23C), complementing the observed increase in tumor-infiltrating CD8+ T cells. CD25 (2%; FIG. 5H and FIGS. 23A-23C) and CD69 (13%; FIG. 5I and FIGS. 23A-23C) expression were both significantly increased in the CD8+ T cells in the spleens of mice treated with PBNP-PTT+aCD137, suggesting that the treatment caused a systemic T cell activation. Splenic T cells of treated mice also showed no change in T cell exhaustion marker, PD-1, compared to CTRL (FIGS. 24A-24B). To further elucidate the T cell response driving abscopal efficacy in SM1 melanoma-bearing mice, splenic T cells were analyzed for CD4+ and CD8+ naïve (CD62Lhi/CD44lo) versus memory (CD62Llo/CD44hi) markers. PBNP-PTT+aCD137 caused a significant decrease in CD4+ naïve (10%; FIG. 5J) and a significant increase in CD4+ memory T cells (36%, FIG. 5K). The same trend was observed in the CD8+ T cell subset (6.4% naïve; 39% memory) (FIGS. 5L-5M; see the gating strategy in FIGS. 25A-25B). These data suggested that not only did the combination nanoimmunotherapy cause DC and T cell activation and infiltration, but also generated a T cell-mediated immunological memory response against SM1 melanoma. A similar pattern of CD4+ and CD8+ memory T cell generation was observed in mice treated with aCD137 monotherapy, although this treatment did not slow tumor growth nor increase survival benefit. These findings suggested the importance of the localized PBNP-PTT in initiating the antitumor response that was then potentiated by aCD137 in the combination nanoimmunotherapy disclosed herein.

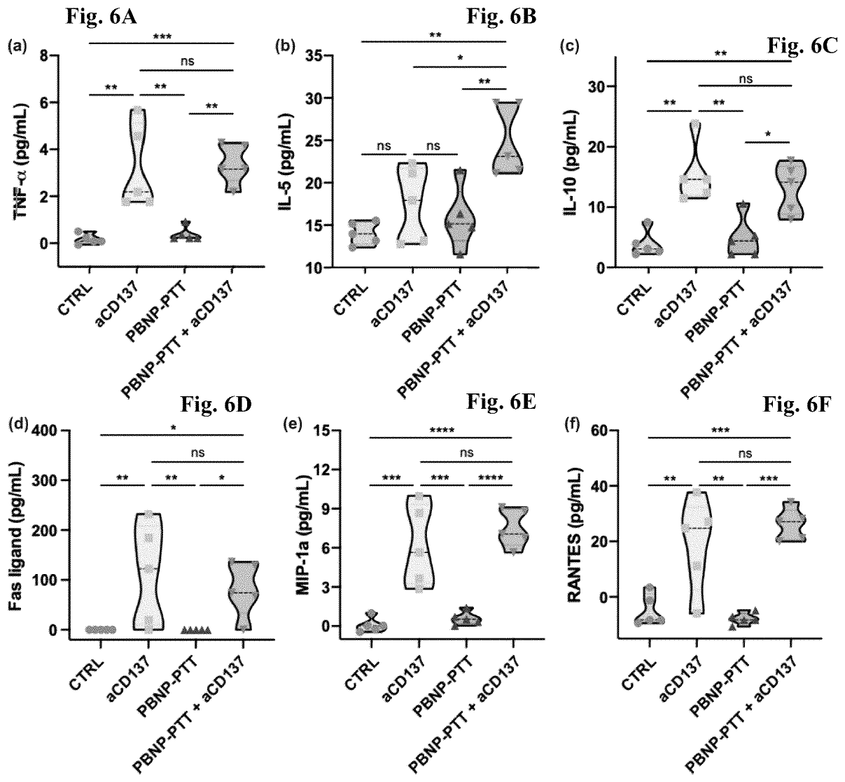

To further characterize the systemic immune response generated by PBNP-PTT+aCD137, serum was harvested from mice 14 days after treatments were initiated and analyzed for cytokines and chemokines associated with T cell-mediated immunity. TNFα, IL-10, Fas ligand, MIP-1a, and RANTES were chosen as representative cytokines/chemokines involved in an inflammatory immune response. IL-5 was investigated as it has shown different immunological functions dependent on its context. Measured by magnetic multiplexing, PBNP-PTT+aCD137 significantly increased serum concentrations of TNFα (4.5 pg/mL), IL-5 (23.5 pg/mL), IL-10 (14.1 pg/mL), Fas ligand (73.4 pg/mL), MIP-1a (7.1 pg/mL), and RANTES (26.8 pg/mL) compared to CTRL (0, 13, 2.8, 0, 0, 0 pg/mL, respectively) and PBNP-PTT treatment (0, 15, 4.3, 0, 0.4, 0 pg/mL, respectively) (FIGS. 6A-6F). TNFα (2.1 pg/mL), IL-10 (14.4 pg/mL), Fas ligand (122.6 pg/mL), MIP-1a (5.6 pg/mL), and RANTES (24.8 pg/mL) were also significantly increased by aCD137 treatment alone. However, PBNP-PTT+aCD137 significantly increased scrum IL-5 expression compared to all other groups (FIG. 6B). These changes in serum cytokines and chemokines suggested the engagement of a systemic antitumor immune effect driving the observed responses. It is important to note that these measurements represented a snapshot in time, based on when the serum was harvested from the animal (14 days after PBNP-PTT and 24 hours after the final aCD137 dose). As such, these results did not represent the dynamic cytokine landscape that may adapt over time.

### Example 6

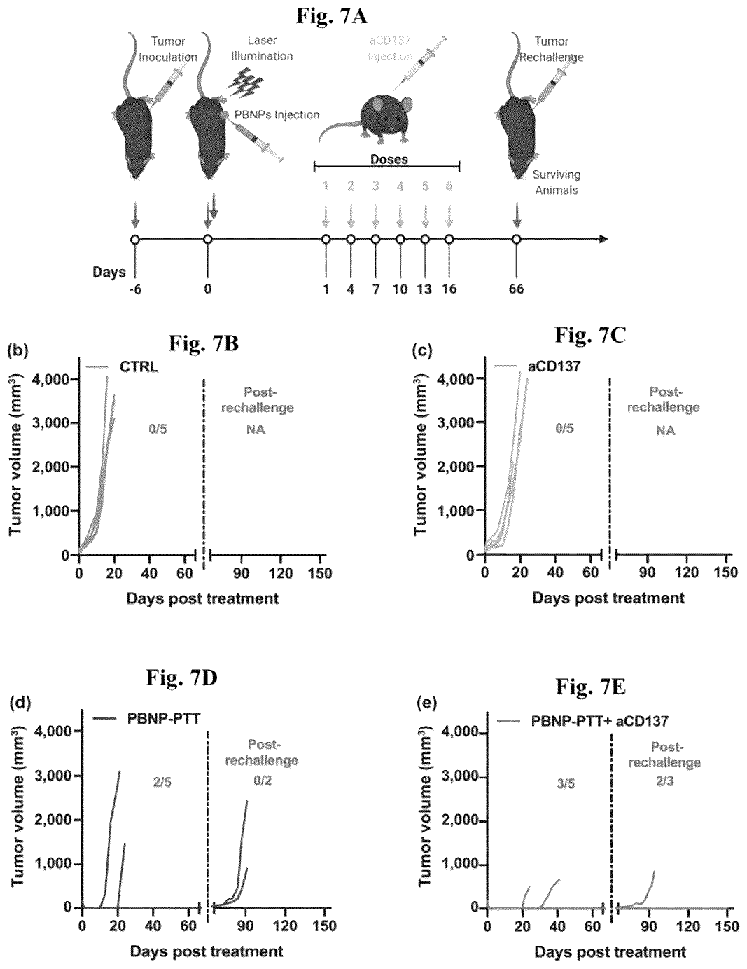

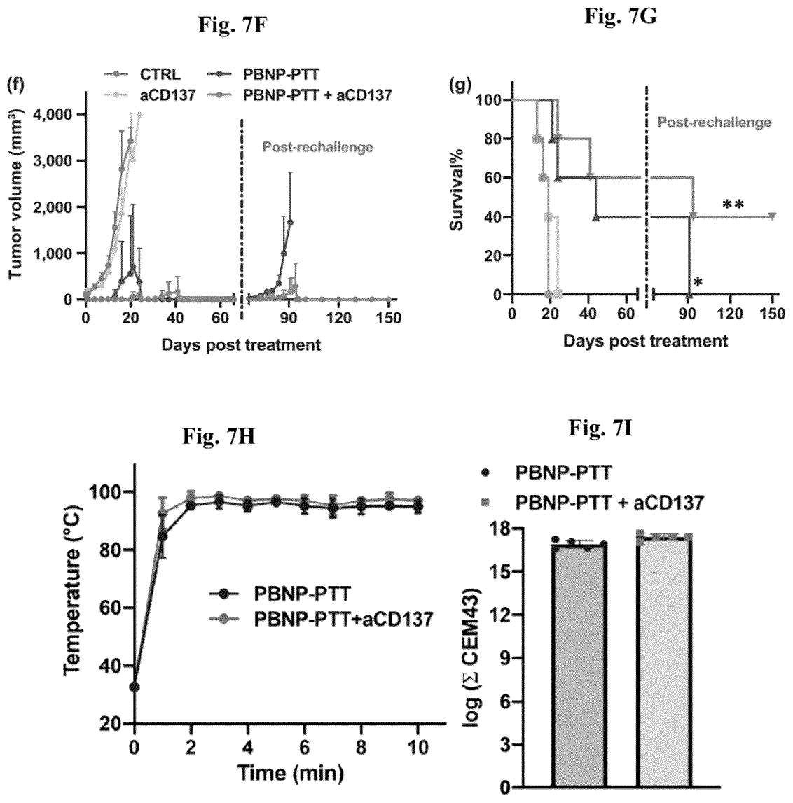

In another exemplary method, PBNP-PTT combined with aCD13 generated T cell-mediated immunological memory. Having established the therapeutic efficacy of PBNP-PTT+aCD137 to treat SM1 melanoma tumors in vivo and generate both CD4+ and CD8+ memory T cells, the ability of the memory T cells to prevent melanoma recurrence in vivo was investigated. To this end, a single SM1 tumor model was utilized in C57BL/6 mice. The nanoimmunotherapy or control treatments were administered to tumor-bearing mice, and the mice were monitored for tumor growth and long-term survival. After completion of the treatments, all long-term surviving animals were rechallenged with SM1 cells (FIGS. 7A and 7H-7I). The aCD137 monotherapy was unable to significantly slow tumor growth compared to CTRL, and thus generated no significant survival benefit (FIGS. 7B-7C). PBNP-PTT eliminated 100% of treated tumors and generated a significant increase in survival; however, 60% of treated mice succumbed to their disease (FIG. 7D). Importantly, PBNP-PTT+aCD137 enabled complete tumor cure and long-term survival in 60% of treated mice (FIG. 7E). Long-term surviving mice treated with PBNP-PTT (n=2) and PBNP-PTT+aCD137 (n=3) were rechallenged with SM1 cells 66 days after the initial treatment to examine immunological memory. Strikingly, 66% of mice treated with PBNP-PTT+aCD137 rejected tumor rechallenge (FIGS. 7E-7F) and exhibited complete tumor-free survival (FIG. 7G), suggesting that immunological memory was established. Naïve age-matched mice and rechallenged PBNP-PTT-treated mice rapidly succumbed to tumor burden (FIGS. 26A-26B). Comparative tumor growth kinetics are illustrated in averaged curves (FIG. 7F). These effects in tumor growth resulted in significantly improved survival benefit for mice treated with PBNP-PTT (MS: 44 days) and PBNP-PTT+aCD137 (MS: not achieved by day of rechallenge) compared to CTRL (MS: 19 days) and aCD137 treatment (MS: 19 days) (FIG. 7G). These data complemented the observations of increased CD4+ and CD8+ memory T cells in the spleens of PBNP-PTT+aCD137-treated mice, and illustrated that antitumor immune memory was generated, manifesting in the rejection of melanoma rechallenge.

### Example 7

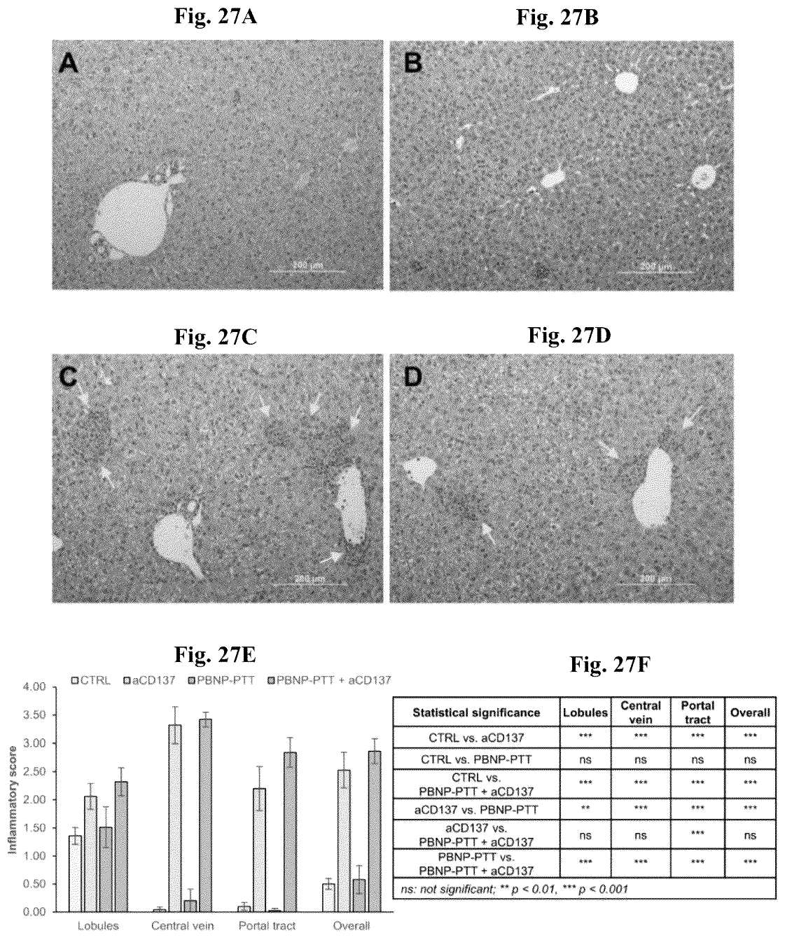

In an exemplary method, PBNP-PTT combined with aCD137 generated acute hepatotoxicity similar to aCD137 monotherapy but appeared to recover over time. Because success of aCD137 therapy relies on reduction of hepatotoxicity, inflammation in harvested livers was examined to quantify acute toxicity 14 days after PBNP-PTT and 24 hours after the final aCD137 i.p. injection. Livers from CTRL and PBNP-PTT-treated animals appeared healthy and non-inflamed (FIGS. 27A-27F). As predicted, aCD137 monotherapy generated acute hepatitis and significant increases in inflammatory foci (90 foci), primarily around the central veins, compared to CTRL (8 foci) and PBNP-PTT-treated animals (12 foci; FIGS. 27A-27F). Livers from PBNP-PTT+aCD137-treated mice also showed increased liver inflammation (142 foci; FIGS. 27A-27F) compared to controls (FIGS. 27A-27F). However, it was predicted that the synergy generated by the combination therapy may enable a lower effective aCD137 dose, thereby mitigating aCD137-mediated hepatotoxicity based on data in the examples herein. It was also noteworthy that this assessment was made 24 hours after injection of aCD137, and therefore it may represent short-term hepatic inflammation.

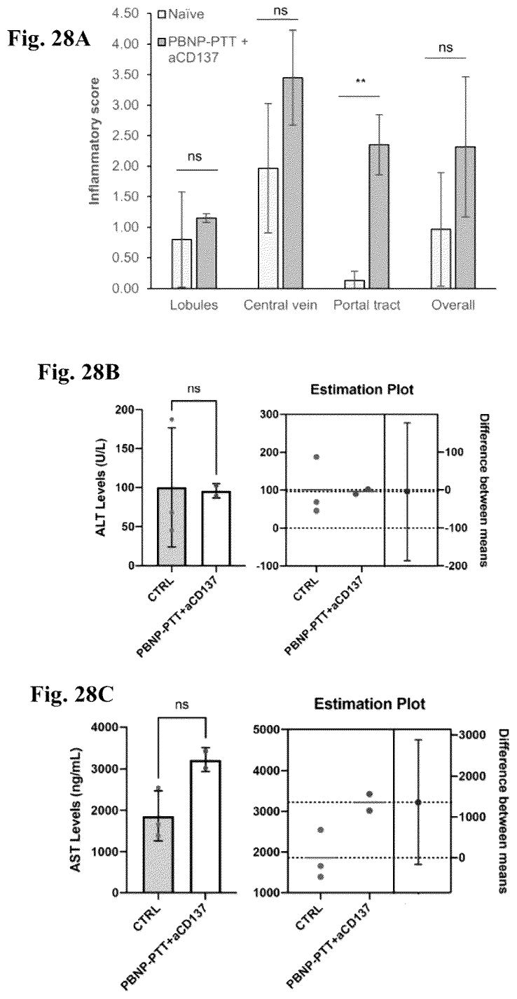

Thus, to investigate the long-term effect of PBNP-PTT+aCD137 on hepatotoxicity, livers from the long-term surviving PBNP-PTT+aCD137-treated mice that had recovered from both the primary SM1 tumor and rechallenged SM1 tumor (226 days post-treatment) were harvested. Although livers from PBNP-PTT+aCD137-treated mice exhibited significantly increased inflammation in the portal tracts compared to age-matched controls, this inflammation was scored as mild, and there was no increased inflammation in the lobules, central veins, or overall (FIGS. 28A-28C). Additionally, serum levels of ALT and AST were measured from these mice at the same timepoint as an indication of liver damage. Long-term surviving PBNP-PTT+aCD137-treated mice had statistically identical serum ALT and AST (FIGS. 28A-28C) levels to their age-matched controls, suggesting normal liver health. These findings indicated that although PBNP-PTT+aCD137 induced acute hepatotoxicity, there were no long-term liver health concerns above normal aging effects.

### Discussion of Examples 1-7

The data presented in Examples 1-7 herein demonstrate a novel nanoimmunotherapy platform for effectively treating melanoma. PBNP-PTT combined with agonistic aCD137 mAb therapy functioned at the interface of nanomedicine and immunotherapy, blending complementary and synergistic immunogenic elements and engaging the beneficial consequences of each monotherapy. At an optimal thermal dose, PBNP-PTT provided robust tumor debulking, increased immunogenicity, and release of tumor-specific immune cell targets, which was potentiated by the ability of aCD137 to engage T cell activation in the context of distal or metastasized melanoma. Accordingly, the examples herein provide for a novel treatment option for melanoma patients, as well as those with other cancers.

### Methods Used in Examples 1-7

Cells. Murine SM1 melanoma cells (BRAFV600E mutation; “SM1”) and human WM9 (“WM9”) and WM793 (“WM793”) melanoma cells (BRAFV600E mutation) were used as indicated in the examples herein. SM1 and WM9 were cultured in Roswell Park Memorial Institute medium (RPMI 1640, Gibco, Carlsbad, CA) containing 1% L-glutamine (pre-supplemented), 10% fetal bovine serum (FBS, Gibco, Carlsbad, CA), and 1% penicillin/streptomycin (Sigma-Aldrich, St. Louis, MO). WM793 was cultured in Dulbecco's modified Eagle medium (DMEM, Gibco, Carlsbad, CA) containing 1% L-glutamine (pre-supplemented), 10% fetal bovine serum (FBS), and 1% penicillin/streptomycin.

PBNP Synthesis. Potassium hexacyanoferrate(II) trihydrate (K₄[Fe(CN)₆]·3H₂O) and iron(III) chloride hexahydrate (FeCl₃·6H₂O) were purchased from Sigma-Aldrich (St. Louis, MO). All synthetic procedures were conducted using ultrapure water obtained from a Milli-Qsystem (Millipore Corporation, Billerica, MA). Prussian blue nanoparticles (“PBNP”) were synthesized using a scheme similar to that of Vojtech et al., J. Vis. Exp. (2015) 28, e52621, the disclosure of which is incorporated herein in its entirety. PBNPs were next characterized for uniformity (FIG. S1). Briefly, an aqueous solution of 54.06 mg of FeCl₃·6H₂O (10 mM) in 20 mL of Milli-Q water was added under vigorous stirring to an aqueous solution containing 84.5 mg of K₄Fe(CN)₆·3H₂O (10 mM) in 20 mL of Milli-Q water. After stirring for 15 minutes, the precipitate was isolated by centrifugation in equal parts water and acetone (10,000 g for 15 minutes) and rinsed by sonication (30 seconds, 40% amplitude) in Milli-Q water. The isolation and rinsing steps were repeated three times before the particles were resuspended by sonication in Milli-Q water.

In vitro PBNP-PTT. Five (5) million SM1 cells were suspended in 500 μL of 1×phosphate buffered saline (PBS). When used, PBNPs were added to the samples at 0.15 mg/mL. The samples were then illuminated by the NIR laser (808 nm; Laserglow Technologies, Toronto, Canada) for 10 minutes at varied power (0.75 W, 1.0 W, 1.5 W, and 2.0 W) to administer PBNP-PTT at various thermal doses/temperature ranges. The maximum temperature of the cell suspension was measured using a thermal camera (forward-looking infrared (FLIR), Arlington, VA), and temperatures were recorded every minute for 10 minutes. Thermal doses were calculated using the following formula:

$$CEM = {\sum }_{i = 1}^{n} {t}_{i} \times {R}^{( 43 - {T}_{i} )} ,$$

where tᵢ is the ith time interval, R=0.25 when T<43° C. and 0.5 when T>43° C., and T is the average temperature during tᵢ.

In vitro Cell Analysis. Following in vitro PBNP-PTT (as described above), cell suspensions were centrifuged and the cell pellet was re-plated in RPMI media in 6-well plates at 37° C. Antibodies were purchased from Biolegend (San Diego, CA), denoted with “#” prior to the catalog number, and Abcam (Cambridge, UK), denoted with “ab” prior to the catalog number. After 24 hours, cells were harvested and stained with Zombie Violet/Green Fixable viability dye (#423114 or #423112), fluorescent antibodies against calreticulin (ab209577), CD80 (#104718), CD86 (#105008), MHC-1 (#116510), CD137L (#107105), PD-L1 (#124315) (surface stains), and high mobility group box-1 protein (HMGB1) (ab195011), and Melan-A/MART1 (ab225500) (intracellular stains). Flow cytometry was performed using the BD Biosciences Celesta Cell Analyzer (Franklin Lakes, NJ), and cytometric analysis was done using FlowJo software (Ashland, OR). For estimation of intracellular ATP, cells were harvested 24 hours after in vitro PBNP-PTT, washed with 1× PBS, and mixed with ATP reagent from the CellTiter-GloLuminescent Cell Viability Assay (Promega, Madison, WI); luminescence was measured using a SpectraMax microplate reader (Molecular Devices, San Jose, CA).

Animals. Five-week-old female C57BL/6 mice were purchased from Jackson Laboratory (Bar Harbor, ME). The animals were acclimated for 7 days prior to handling. Tumor volumes (mm³) were monitored every other day by caliper measurement and calculated using the formula: (long diameter×short diameter²)/2. A tumor size of 20 mm in diameter in any dimension for single tumors, or 15 mm in any dimension if two tumors were present, was designated as the endpoint, and mice were euthanized at that time (if not earlier as designated in study design). Euthanasia was achieved through cervical dislocation after CO₂ narcosis. If tumors impaired the mobility of the animal, became ulcerated, or appeared infected, or if the mice displayed signs of distress, exhibiting a sick mouse posture, the mouse was euthanized.

Ex vivo Co-culture Analysis. To assess the effects of PBNP-PTT treatment of SM1 cells on direct T cell activation, spleens were harvested from tumor-free C57BL/6 mice after euthanasia. Splenic T cells were magnetically isolated by negative selection using Pan T cell isolation Kit II mouse (Miltenyi Biotec, Bergisch Gladbach, Germany), and subsequently co-cultured with SM1 cells pre-treated with PBNP-PTT at varied laser power (1 W, 1.5 W, and 2 W) for 24 or 48 hours in TexMACS medium (Miltenyi Biotec). Cells were then harvested and stained with Zombie Red Fixable viability dye (#423109) and fluorescent antibodies to detect CD3(#100236), CD4 (#100443), CD8a (#100723), CD25 (#101917), and CD69 (#104539) (Biolegend).

Tumor Inoculation. The backs of five-week-old female C57BL/6 mice were shaved prior to tumor cell inoculation. For the single tumor model, 1 million SM1 cells were inoculated into the backs of C57BL/6 mice. For two-tumor models, two injections of 1 million SM1 cells were inoculated simultaneously (synchronous model) or four days apart (metachronous model). Long-term surviving animals were rechallenged by inoculating 1 million SM1 cells into the mice 66 days post-treatment. Tumor growth was regularly monitored and measured by calipers.

In vivo PBNP-PTT and mAb treatments. When tumors became palpable (˜60 mm³), mice were randomly divided into treatment groups. Mice were anesthetized prior to and during treatment using 2%-5% isoflurane. Tumors receiving PBNP-PTT were intratumorally injected with 2.5 mg/kg PBNPs. Following injections, tumors were irradiated with the NIR laser (808 nm; Laserglow Technologies, Toronto, ON, Canada) for 10 minutes, and temperatures were measured by thermal camera at one-minute intervals (FLIR, Arlington, VA). Eyes were covered with opaque black cardboard during treatment to avoid eye damage by the laser. Mice receiving mAb treatments were intraperitoneally (i.p.) injected with 5, 10, or 15 mg/kg anti-CTLA-4 (clone 9D9), 15 mg/kg anti-PD-1 (clone RMP1-14), or 15 mg/kg anti-CD137 (clone 3H3) (all mAbs were purchased from BioXCell, WestLebanon, NH) every three days for two weeks (i.e., on days 1, 4, 7, 10, 13, 16, totaling 6 doses). After administering the individual treatments, the mice were monitored for tumor progression and survival.

Ex vivo Immunological Analysis. All antibodies were purchased from Biolegend unless otherwise stated. Synchronous tumor-bearing mice were euthanized 14 days after initial treatment, and their secondary tumors, spleens, lymph nodes, blood, and livers were harvested for further analysis. Secondary tumors were cut into smaller sections and digested using a tissue digestion mixture (1 gram (g) of Collagenese I (Sigma-Aldrich, C0130), 1 g of Collagenase IV (Sigma-Aldrich, C5138), 0.5 g of Hyaluronidase V (Sigma-Aldrich, H6254), 0.2 g of DNAase I, and Pierce Protease Inhibitor, ethylenediaminetetraacetic acid (EDTA) free (ThermoFisher, Waltham, MA; A32965) in 200 mL of Hank's Balanced Salts Solution (HBSS) (Sigma-Aldrich, 55021C)), followed by preparation of single cell suspensions by passing the digested tumors through a series of 100 and 80 μm cell strainers. One (1) million cells from each tumor sample were then stained with ZombieAqua Fixable viability dye (#423102) and antibodies against CD3 (#147501 kit), CD4 (#147501 kit), and CD8 (#100737). Single cell suspensions of splenic cells were prepared by passing the isolated spleens through a 100 μm cell strainer using a plunger. Cells were centrifuged, and the cell pellet was suspended in ammonium-chloride-potassium (ACK) lysis buffer (ThermoFisher) to lyse red blood cells. Splenic T cells were magnetically isolated by negative selection using a mouse Pan T cell isolation Kit II (Miltenyi Biotec). They were then stained with a Zombie Aqua Fixable viability dye and antibodies against CD3 (#147501 kit), CD4 (#147501 kit), CD8 (#100737), CD69 (#104508), CD25 (#102020), PD-L1 (#135214), CD44 (#147501kit), and CD62L (#147501 kit). Lymph nodes were cut into small sections and single cell suspensions were prepared by passing the isolated lymph nodes through 100 μm cell strainers using a plunger. The cells were then counted and stained using a ZombieAqua Fixable viability dye and antibodies against CD11c (#117320), CD80 (#104714), and CD86 (#105007). Samples were then analyzed by flow cytometry using the FlowJo software. Blood was collected post-euthanasia and allowed to clot (first on ice, then at room temperature for 30 minutes) and then centrifuged to separate cells from serum. Serum was then collected and analyzed for cytokine/chemokine content using a custom magnetic bead-based Multiplexing Assay kit from R&D Systems and the LuminexMAGPIX Instrument with xPOTENT 4.3 software. When analyzed, values that fell below the detectable range of the Luminex Instrument were recorded as 0 pg/mL.

Liver Histology. Harvested livers were fixed in 4% paraformaldehyde (PFA) and embedded in paraffin wax, followed by sectioning using a microtome, and staining with hematoxylin and eosin (H&E). Tissues were analyzed by a trained medical pathologist. The number of tumor metastatic infiltrates was recorded as clusters of greater than 10 neoplastic cells. Individual cells or smaller clusters in sinusoids were not included. Inflammatory scoring was evaluated using a method adapted from Bartkowiak et al., Clin. Cancer Res. (2018) 24, 1138-1151 the disclosure of which is incorporated herein in its entirety. Hepatic inflammation was evaluated in five liver tissue samples per animal, encompassing multiple lobes. Three H&E-stained tissue sections were evaluated for each animal. The slide with the greatest evidence of inflammation for each mouse was selected for more detailed analysis. All tissue sections on the slide were scanned for clusters of inflammatory cells (foci). The number of inflammatory cells was counted in each focus within three areas of the liver, including portal tracts, central veins, and lobular parenchyma (lobules). Each of the three areas of liver was analyzed for the number of inflammatory foci. Each inflammatory focus was assigned an inflammatory score of 0-4 dependent on the number of inflammatory cells in the focus (1: minimal (<20 cells); 2: mild (21-50 cells); 3: moderate (51-150 cells); 4: severe (>150 cells)). The average score and standard deviation were calculated of the 10 most affected foci were determined for each animal. One mouse in the age-matched naïve control group appeared to have signs of fatty liver disease.

Liver Enzyme Analysis. Blood from long-term surviving PBNP-PTT+aCD137-treated mice (n=2) and naïve tumor-free age-matched C57BL/6 mice (n=3) was obtained post-euthanasia and allowed to clot (first on ice, then at room temperature for 30 minutes) and then centrifuged to separate cells from serum. Serum was then carefully collected and analyzed for alanine aminotransferase (ALT) (GTP Assay Kit (Colorimetric); Catalog #KA1294, Novus Biologicals LLC, Centennial CO, USA) and aspartate aminotransferase (AST) (Mouse AST SimpleStep ELISA Kit, ab263882, Abcam, Cambridge, UK) using protocols provided by the manufacturers.

Statistics. Statistical significance was determined by one-way ANOVA using Tukey's multiple comparison test. Statistics on tumor growth curves were determined by two-way ANOVA using Tukey's multiple comparison test and multiple t test, comparing averaged tumor volumes between each individual time point. Survival results were analyzed according to a Kaplan-Meier curve. The log-rank (Mantel-Cox) test was used to determine statistically significant differences in survival between groups. Values were considered statistically significantly different when p values were less than 0.05.

### *******************

All the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating a subject, the method comprising:
(i) administering to a subject having cancer a biofunctionalized nanocomposite, wherein the biofunctionalized nanocomposite comprises
a core comprising a nanoparticle formed of Prussian blue materials;
a shell obtained by partially or completely encapsulating the Prussian blue core with a biocompatible coating; and
at least one anti-CD137 antibody attached to, or absorbed to, the biocompatible coating; and
(ii) subjecting the subject to photothermal therapy.

2. The method of claim 1, wherein the Prussian blue materials are iron hexacyanoferrate (II) compounds.

3. The method of claim 1, wherein the Prussian blue materials are represented by general formula (II):
AxFeYIII[FeII(CN)6]z·nH2O (II)
wherein:
A represents at least one of Li, Na, K, Rb, Cs, NH4 and Tl in any oxidation state and any combination thereof;
X is from 0 to about 1;
Y is from 0.1 to about 4;
Z is from 0.1 to about 4; and
N is from 1 to about 24.

4. The method of claim 1, wherein the biocompatible coating of the shell comprises at least one member selected from the group consisting of dextran, chitosan, silica, polyethylene glycol (PEG), avidin; a protein, a nucleic acid, a carbohydrate, a lipid, neutravidin, streptavidin, gelatin, collagen, fibronectin, albumin, a serum protein, a lysozyme, a phospholipid, a polyvinyl pyrrolidone (PVP), a polyvinyl alcohol, polyethylene glycol diacrylate, and polyethylenimine (PEI).

5. The method of claim 4, wherein the biocompatible coating of the shell comprises polyethylene glycol (PEG), a polyvinyl pyrrolidone (PVP), a polyvinyl alcohol, polyethylenimine (PEI), or a combination thereof.

6. The method of claim 1, wherein the anti-CD137 antibody is selected from the group consisting of BMS-663513, AGEN2373, ADG106, EU101, and PF-05082566.

7. The method of claim 1, wherein the biocompatible coating further comprises at least one imaging agent.

8. The method of claim 7, wherein the at least one imaging agent is selected from the group consisting of a fluorescein compound, a rhodamine compound, a xanthene compound, a cyanine compound, a naphthalene compound, a coumarin compound, an oxadiazole compound, a pyrene compound, an oxazine compound, an acridine compound, an arylmethine compound, and a tetrapyrrole compound.

9. The method of claim 1, wherein the photothermal therapy comprises using a device that emits electromagnetic radiation at a wavelength capable of irradiating the biofunctionalized nanocomposite.

10. The method of claim 9, wherein the wavelength capable of irradiating the biofunctionalized nanocomposite comprises from about 600 nm to about 1000 nm.

11. The method of claim 1, wherein subjecting the subject to photothermal therapy comprises about 4 minutes to about 20 minutes.

12. The method of claim 1, further comprising repeating steps (i) and (ii) at least once a week for at least 3 weeks.

13. The method of claim 1, wherein the cancer is an immune checkpoint inhibitor-resistant cancer.

14. The method of claim 1, wherein the subject has received prior treatment with one or more immune checkpoint inhibitors.

15. The method of claim 1, further comprising administering to the subject one or more monoclonal antibodies selected from the group consisting of adotrastuzumab, alemtuzumab, atezolizumab, avelumab, bevacizumab, blinatumomab, brentuximab, cemiplimab, cetuximab, daratumumab, denosumab, dinutuximab, durvalumab, elotuzumab, gemtuzumab, ibritumomab, inotuzumab, ipilimumab, necitumumab, nivolumab, obinutuzumab, ofatumumab, olaratumab, panitumumab, pembrolizumab, pertuzumab, ramucirumab, rituximab, tositumomab, and trastuzumab.

16. The method of claim 1, wherein, following photothermal therapy, the biofunctionalized nanocomposite induces a cytotoxic T lymphocyte response.

17. The method of claim 1, wherein, following photothermal therapy, the metastasis of at least one tumor cell in the subject is prevented.

18. The method of claim 1, wherein, following photothermal therapy, one or more tumors in the subject are sensitized to an anti-cancer drug.

19. The method of claim 1, wherein, following photothermal therapy, one or more symptoms associated with the cancer are treated in the subject.

20. The method of claim 19, wherein the one or more symptoms associated with the cancer comprise anemia, loss of appetite, irritation of bladder lining, bleeding and bruising, changes in taste or smell, constipation, diarrhea, dry mouth, dysphagia, edema, fatigue, hair loss, infection, infertility, lymphedema, mouth sores, nausea, pain, peripheral neuropathy, tooth decay, urinary tract infections, problems with memory and concentration, or any combination thereof.
